SEQUENCE LISTING (334 sequences)

SEQ ID NO: 1 - DNA, 18 bp, Artificial Sequence
  Synthetic DNA/RNA hybrid oligonucleotide
acggaattcctcactaaa

SEQ ID NO: 2 - DNA, 17 bp, Artificial Sequence
  Synthetic oligonucleotide primer
attgatggtgcctacag

SEQ ID NO: 3 - DNA, 24 bp, Artificial Sequence
  Synthetic oligonucleotide primer
cagccaacggaattcctcactaaa

SEQ ID NO: 4 - DNA, 30 bp, Artificial Sequence
  Synthetic oligonucleotide primer
gagccaacaggcaccgaattcctcactaaa

SEQ ID NO: 5 - DNA, 22 bp, Artificial Sequence
  Synthetic oligonucleotide primer
tgagctacagtgcttcatctca

SEQ ID NO: 6 - DNA, 22 bp, Artificial Sequence
  Synthetic oligonucleotide primer
tcaacatcagtctgataagcta

SEQ ID NO: 7 - RNA, 23 nt, Homo sapiens
auauaauacaaccugcuaagugu

SEQ ID NO: 8 - RNA, 64 nt, Homo sapiens
cggauggauauaauacaaccugcuaaguguccuagcacuuagcagguuguauuaucauuguccg

SEQ ID NO: 9 - RNA, 22 nt, Homo sapiens
ugugcgcagggagaccucuccc

SEQ ID NO: 10 - RNA, 77 nt, Homo sapiens
acuugggucaguucagagguccucggggcgcgcgucgagucagccgugugcgcagggagaccucucccacccacagu

SEQ ID NO: 11 - RNA, 22 nt, Homo sapiens
cugggaucuccggggucuuggu

SEQ ID NO: 12 - RNA, 87 nt, Homo sapiens
ccugggcucugaccugagaccucuggguucugagcugugauguugcucucgagcugggaucuccggggucuugguucagggccgggg

SEQ ID NO: 13 - RNA, 24 nt, Homo sapiens
aggaagcccuggaggggcuggagg

SEQ ID NO: 14 - RNA, 84 nt, Homo sapiens
ggccaggaagaggaggaagcccuggaggggcuggaggugauggauguuuuccuccgguucucagggcuccaccucuuucgggcc

SEQ ID NO: 15 - RNA, 22 nt, Homo sapiens
ugucuacuacuggagacacugg

SEQ ID NO: 16 - RNA, 83 nt, Homo sapiens
agaaauaaggcuucugucuacuacuggagacacugguaguauaaaacccagagucuccaguaauggacgggagccuuauuucu

SEQ ID NO: 17 - RNA, 23 nt, Homo sapiens
ccaguuaccgcuuccgcuaccgc

SEQ ID NO: 18 - RNA, 91 nt, Homo sapiens
ggcgggggcgcgggcggcaguggcgggagcggccccucggccauccuccgucugcccaguuaccgcuuccgcuaccgccgccgcucccgcu

SEQ ID NO: 19 - RNA, 22 nt, Homo sapiens
acaguagagggaggaaucgcag

SEQ ID NO: 20 - RNA, 98 nt, Homo sapiens
ucaaggccacugggacaguagagggaggaaucgcagaaaucacuccaggagcaacugagagaccuugcuucuacuuuaccagguccugcuggcccaga

SEQ ID NO: 21 - RNA, 22 nt, Homo sapiens
auccgcgcucugacucucugcc

SEQ ID NO: 22 - RNA, 86 nt, Homo sapiens
agcacugcccccggugagucaggguggggcuggcccccugcuucgugcccauccgcgcucugacucucugcccaccugcaggagcu

SEQ ID NO: 23 - RNA, 22 nt, Homo sapiens
ugcccuuaaaggugaacccagu

SEQ ID NO: 24 - RNA, 83 nt, Homo sapiens
gaagguguaccaugugcccuuaaaggugaacccagugcaccuucaugaaccgugguacaccuuuaagaacuugguaugccuuc

SEQ ID NO: 25 - RNA, 24 nt, Homo sapiens
uggggagcugaggcucugggggug

SEQ ID NO: 26 - RNA, 82 nt, Homo sapiens
ugugggcagggcccuggggagcugaggcucuggggguggccggggcugacccugggccucugcuccccagugucugaccgcg

SEQ ID NO: 27 - RNA, 21 nt, Homo sapiens
aaggcagggcccccgcucccc

SEQ ID NO: 28 - RNA, 94 nt, Homo sapiens
gugaggugugggcccggccccaggagcggggccugggcagccccguguguugaggaaggaaggcagggcccccgcuccccgggccugaccccac

SEQ ID NO: 29 - RNA, 23 nt, Homo sapiens
cacccggcugugugcacaugugc

SEQ ID NO: 30 - RNA, 89 nt, Homo sapiens
cccggcuguguggacaugugcccagggcccgggacagcgccacggaagaggacgcacccggcugugugcacaugugcccagggcccggg

SEQ ID NO: 31 - RNA, 22 nt, Homo sapiens
ucuucucuguuuuggccaugug

SEQ ID NO: 32 - RNA, 86 nt, Homo sapiens
auuaggagaguaucuucucuguuuuggccauguguguacucacagccccucacacauggccgaaacagagaaguuacuuuccuaau

SEQ ID NO: 33 - RNA, 21 nt, Homo sapiens
cugacuguugccguccuccag

SEQ ID NO: 34 - RNA, 94 nt, Homo sapiens
gggacguucugagcucggggugggggacguuugccggucacugcugcuggcgcccugacuguugccguccuccagccccacucaaaggcauccc

SEQ ID NO: 35 - RNA, 22 nt, Homo sapiens
aaauuauuguacaucggaugag

SEQ ID NO: 36 - RNA, 88 nt, Homo sapiens
guuccagacacaucucaucugauauacaauauuuucuuaaauuguauaaagagaaauuauuguacaucggaugagcugugucugggau

SEQ ID NO: 37 - RNA, 22 nt, Homo sapiens
aaggagcuuacaaucuagcugg

SEQ ID NO: 38 - RNA, 93 nt, Homo sapiens
gguaacugcccucaaggagcuuacaaucuagcuggggguaaaugacuugcacaugaacacaacuagacugugagcuucuagagggcagggacc

SEQ ID NO: 39 - RNA, 22 nt, Homo sapiens
cggccccacgcaccaggguaag

SEQ ID NO: 40 - RNA, 21 nt, Homo sapiens
cugcccuggcccgagggaccg

SEQ ID NO: 41 - RNA, 61 nt, Homo sapiens
cugcggccccacgcaccaggguaagagagacucucgcuuccugcccuggcccgagggaccg

SEQ ID NO: 42 - RNA, 22 nt, Homo sapiens
cgaggaggcucccagagugugu

SEQ ID NO: 43 - RNA, 81 nt, Homo sapiens
uccuggugagccgaggaggcucccagaguguguaugguguggaucagacucacucagggcucccccugcaucacaucagga

SEQ ID NO: 44 - RNA, 21 nt, Homo sapiens
agcgaugugggaaggcuugug

SEQ ID NO: 45 - RNA, 90 nt, Homo sapiens
cugcccugcucuggccuuccuuucccaccccgcuggguuugcaucugcaggugggagcgaugugggaaggcuuguggaggagcagcucag

SEQ ID NO: 46 - RNA, 21 nt, Homo sapiens
acucuuagcgguggaucacuc

SEQ ID NO: 47 - RNA, 85 nt, Homo sapiens
gugccaggagggccucccgguguugggagucccucaccaaagaacuuugugaaacucuuagcgguggaucacuccacuccugcau

SEQ ID NO: 48 - RNA, 24 nt, Homo sapiens
cagaugaggaagccauggcuaguu

SEQ ID NO: 49 - RNA, 87 nt, Homo sapiens
ugagaggagagaggccaggaaaauuauuuaaagaagccaaccauucaggcaacagaugaggaagccauggcuaguucuuuuguuuua

SEQ ID NO: 50 - RNA, 22 nt, Homo sapiens
acccgucccguucguccccgga

SEQ ID NO: 51 - RNA, 68 nt, Homo sapiens
gggcgcacccgucccguucguccccggacguugcucucuaccccgggaacgucgagacuggagcgccc

SEQ ID NO: 52 - RNA, 20 nt, Homo sapiens
cucagggugacggugagcag

SEQ ID NO: 53 - RNA, 90 nt, Homo sapiens
guggguaacucagggugacggugagcagugccacucucauuuuacaaguaacucagggugauggugagcagugccacucucauuuuccac

SEQ ID NO: 54 - RNA, 20 nt, Homo sapiens
cccagaugguuccaguuugu

SEQ ID NO: 55 - RNA, 74 nt, Homo sapiens
caaaauggagcuauagcaucuuuuugaguccagugaaaauauaaacagacuccucccagaugguuccaguuugu

SEQ ID NO: 56 - RNA, 22 nt, Homo sapiens
cauaaaugaauggcuggggcaa

SEQ ID NO: 57 - RNA, 64 nt, Homo sapiens
gccuggccugggcagucaccaggauuaugaucucuuucccauaaaugaauggcuggggcaaggc

SEQ ID NO: 58 - RNA, 22 nt, Homo sapiens
ccacuaguaugaucagagccuc

SEQ ID NO: 59 - RNA, 94 nt, Homo sapiens
ggaggucaccccacccccacuaguaugaucagagccuccugugggacccaggcugcgcugaugucuucauggggagaugugcacacgggcgucc

SEQ ID NO: 60 - RNA, 18 nt, Homo sapiens
aacugaggaugggaaagc

SEQ ID NO: 61 - RNA, 23 nt, Homo sapiens
auaagcccaggaaauguguagag

SEQ ID NO: 62 - RNA, 18 nt, Homo sapiens
ccaagcgagggaacagug

SEQ ID NO: 63 - RNA, 19 nt, Homo sapiens
uggaguaagccggaucgcg

SEQ ID NO: 64 - RNA, 20 nt, Homo sapiens
ccuccuggcgggcagcugug

SEQ ID NO: 65 - RNA, 20 nt, Homo sapiens
cgcgggugcuuacugacccu

SEQ ID NO: 66 - RNA, 23 nt, Homo sapiens
cgggucggaguuagcucaagcgg

SEQ ID NO: 67 - RNA, 23 nt, Homo sapiens
cucucugccccugggagagaucc

SEQ ID NO: 68 - RNA, 20 nt, Homo sapiens
gaggugggaaagggaagggu

SEQ ID NO: 69 - RNA, 24 nt, Homo sapiens
uagaggagauggcgcaggggacac

SEQ ID NO: 70 - RNA, 21 nt, Homo sapiens
gugguggugggggaggaggaa

SEQ ID NO: 71 - RNA, 21 nt, Homo sapiens
ucaaggagcucacaaucuagu

SEQ ID NO: 72 - RNA, 20 nt, Homo sapiens
ucugccucaacuccgccccu

SEQ ID NO: 73 - RNA, 22 nt, Homo sapiens
uggaggcaggaugacacuggga

SEQ ID NO: 74 - RNA, 21 nt, Homo sapiens
ugugagauugguggaaaggaa

SEQ ID NO: 75 - RNA, 21 nt, Homo sapiens
cuugguuucccuuuuccuauc

SEQ ID NO: 76 - RNA, 21 nt, Homo sapiens
gcuggugagugacugaauuga

SEQ ID NO: 77 - RNA, 21 nt, Homo sapiens
ccagaaacagaugguggguaa

SEQ ID NO: 78 - RNA, 20 nt, Homo sapiens
cuucaguuuccuugugugca

SEQ ID NO: 79 - RNA, 21 nt, Homo sapiens
cuauugguggcgcuugaauac

SEQ ID NO: 80 - RNA, 22 nt, Homo sapiens
caggugcuuccaaggagccagg

SEQ ID NO: 81 - RNA, 22 nt, Homo sapiens
ccacaucacaacacaugcauag

SEQ ID NO: 82 - RNA, 21 nt, Homo sapiens
ccucugaaauggaugggaccu

SEQ ID NO: 83 - RNA, 24 nt, Homo sapiens
ccuaaaauugugagugcauaucuc

SEQ ID NO: 84 - RNA, 25 nt, Homo sapiens
ccugagugaugggcaugccuuuguc

SEQ ID NO: 85 - RNA, 23 nt, Homo sapiens
cuaaucucuucugaugggcucuu

SEQ ID NO: 86 - RNA, 25 nt, Homo sapiens
cagugaaauugaugugccuuuguuu

SEQ ID NO: 87 - RNA, 22 nt, Homo sapiens
guaauaucagaaguuugggagg

SEQ ID NO: 88 - RNA, 24 nt, Homo sapiens
cguggcuauuugaugagugugcac

SEQ ID NO: 89 - RNA, 24 nt, Homo sapiens
cccuaguugauguggaugauuguc

SEQ ID NO: 90 - RNA, 21 nt, Homo sapiens
cccuguauuuggaugguuuuc

SEQ ID NO: 91 - RNA, 22 nt, Homo sapiens
ccucaugagggagaguaccccc

SEQ ID NO: 92 - RNA, 21 nt, Homo sapiens
ccucaauauagguggcuguau

SEQ ID NO: 93 - RNA, 23 nt, Homo sapiens
auauaauacaaccugcuaagugu

SEQ ID NO: 94 - RNA, 22 nt, Homo sapiens
ugugcgcagggagaccucuccc

SEQ ID NO: 95 - RNA, 22 nt, Homo sapiens
cugggaucuccggggucuuggu

SEQ ID NO: 96 - RNA, 24 nt, Homo sapiens
aggaagcccuggaggggcuggagg

SEQ ID NO: 97 - RNA, 22 nt, Homo sapiens
ugucuacuacuggagacacugg

SEQ ID NO: 98 - RNA, 23 nt, Homo sapiens
ccaguuaccgcuuccgcuaccgc

SEQ ID NO: 99 - RNA, 24 nt, Homo sapiens
aagggcuuuuagggcagguaggug

SEQ ID NO: 100 - RNA, 22 nt, Homo sapiens
acaguagagggaggaaucgcag

SEQ ID NO: 101 - RNA, 22 nt, Homo sapiens
auccgcgcucugacucucugcc

SEQ ID NO: 102 - RNA, 22 nt, Homo sapiens
ugcccuuaaaggugaacccagu

SEQ ID NO: 103 - RNA, 24 nt, Homo sapiens
uggggagcugaggcucugggggug

SEQ ID NO: 104 - RNA, 21 nt, Homo sapiens
aaggcagggcccccgcucccc

SEQ ID NO: 105 - RNA, 23 nt, Homo sapiens
cacccggcugugugcacaugugc

SEQ ID NO: 106 - RNA, 22 nt, Homo sapiens
ucuucucuguuuuggccaugug

SEQ ID NO: 107 - RNA, 21 nt, Homo sapiens
cugacuguugccguccuccag

SEQ ID NO: 108 - RNA, 22 nt, Homo sapiens
ugagguaguagguuguauaguu

SEQ ID NO: 109 - RNA, 22 nt, Homo sapiens
ugagguaguagguugugugguu

SEQ ID NO: 110 - RNA, 21 nt, Homo sapiens
cuauacaaccuacugccuucc

SEQ ID NO: 111 - RNA, 22 nt, Homo sapiens
ugagguaguagguuguaugguu

SEQ ID NO: 112 - RNA, 21 nt, Homo sapiens
agagguaguagguugcauagu

SEQ ID NO: 113 - RNA, 21 nt, Homo sapiens
ugagguaggagguuguauagu

SEQ ID NO: 114 - RNA, 22 nt, Homo sapiens
ugagguaguagauuguauaguu

SEQ ID NO: 115 - RNA, 21 nt, Homo sapiens
ugagguaguaguuuguacagu

SEQ ID NO: 116 - RNA, 21 nt, Homo sapiens
ugagguaguaguuugugcugu

SEQ ID NO: 117 - RNA, 21 nt, Homo sapiens
ugcgcaagcuacugccuugcu

SEQ ID NO: 118 - RNA, 22 nt, Homo sapiens
uggaagacuagugauuuuguug

SEQ ID NO: 119 - RNA, 23 nt, Homo sapiens
uacccuguagauccgaauuugug

SEQ ID NO: 120 - RNA, 22 nt, Homo sapiens
uacccuguagaaccgaauuugu

SEQ ID NO: 121 - RNA, 22 nt, Homo sapiens
uagcagcacauaaugguuugug

SEQ ID NO: 122 - RNA, 22 nt, Homo sapiens
uagcagcacaucaugguuuaca

SEQ ID NO: 123 - RNA, 30 nt, Homo sapiens
ucaagaugcgaaucauuauuugcugcucua

SEQ ID NO: 124 - RNA, 22 nt, Homo sapiens
uagcagcacguaaauauuggcg

SEQ ID NO: 125 - RNA, 24 nt, Homo sapiens
caaagugcuuacagugcagguagu

SEQ ID NO: 126 - RNA, 20 nt, Homo sapiens
acugcagugaaggcacuugu

SEQ ID NO: 127 - RNA, 22 nt, Homo sapiens
uaaggugcaucuagugcagaua

SEQ ID NO: 128 - RNA, 22 nt, Homo sapiens
acugcccuaagugcuccuucug

SEQ ID NO: 129 - RNA, 23 nt, Homo sapiens
ugugcaaaucuaugcaaaacuga

SEQ ID NO: 130 - RNA, 23 nt, Homo sapiens
ugugcaaauccaugcaaaacuga

SEQ ID NO: 131 - RNA, 23 nt, Homo sapiens
uaaagugcuuauagugcagguag

SEQ ID NO: 132 - RNA, 23 nt, Homo sapiens
caaagugcucauagugcagguag

SEQ ID NO: 133 - RNA, 23 nt, Homo sapiens
uagcuuaucagacugauguugac

SEQ ID NO: 134 - RNA, 22 nt, Homo sapiens
caacaccagucgaugggcuguc

SEQ ID NO: 135 - RNA, 22 nt, Homo sapiens
aagcugccaguugaagaacugu

SEQ ID NO: 136 - RNA, 21 nt, Homo sapiens
aucacauugccagggauuucc

SEQ ID NO: 137 - RNA, 22 nt, Homo sapiens
gggguuccuggggaugggauuu

SEQ ID NO: 138 - RNA, 21 nt, Homo sapiens
aucacauugccagggauuacc

SEQ ID NO: 139 - RNA, 22 nt, Homo sapiens
uggguuccuggcaugcugauuu

SEQ ID NO: 140 - RNA, 22 nt, Homo sapiens
uggcucaguucagcaggaacag

SEQ ID NO: 141 - RNA, 22 nt, Homo sapiens
cauugcacuugucucggucuga

SEQ ID NO: 142 - RNA, 21 nt, Homo sapiens
uucaaguaauccaggauaggc

SEQ ID NO: 143 - RNA, 22 nt, Homo sapiens
uucaaguaauucaggauagguu

SEQ ID NO: 144 - RNA, 21 nt, Homo sapiens
uucacaguggcuaaguuccgc

SEQ ID NO: 145 - RNA, 21 nt, Homo sapiens
uucacaguggcuaaguucugc

SEQ ID NO: 146 - RNA, 22 nt, Homo sapiens
gagcuuagcugauuggugaaca

SEQ ID NO: 147 - RNA, 22 nt, Homo sapiens
aaggagcucacagucuauugag

SEQ ID NO: 148 - RNA, 22 nt, Homo sapiens
cacuagauugugagcuccugga

SEQ ID NO: 149 - RNA, 21 nt, Homo sapiens
uagcaccaucugaaaucgguu

SEQ ID NO: 150 - RNA, 23 nt, Homo sapiens
uagcaccauuugaaaucaguguu

SEQ ID NO: 151 - RNA, 21 nt, Homo sapiens
gcugguuucauauggugguuu

SEQ ID NO: 152 - RNA, 20 nt, Homo sapiens
uagcaccauuugaaaucggu

SEQ ID NO: 153 - RNA, 22 nt, Homo sapiens
uguaaacauccucgacuggaag

SEQ ID NO: 154 - RNA, 22 nt, Homo sapiens
uguaaacauccuacacucagcu

SEQ ID NO: 155 - RNA, 23 nt, Homo sapiens
uguaaacauccuacacucucagc

SEQ ID NO: 156 - RNA, 22 nt, Homo sapiens
uguaaacauccccgacuggaag

SEQ ID NO: 157 - RNA, 22 nt, Homo sapiens
uguaaacauccuugacuggaag

SEQ ID NO: 158 - RNA, 21 nt, Homo sapiens
ggcaagaugcuggcauagcug

SEQ ID NO: 159 - RNA, 23 nt, Homo sapiens
uggcagugucuuagcugguuguu

SEQ ID NO: 160 - RNA, 21 nt, Homo sapiens
uauugcacuugucccggccug

SEQ ID NO: 161 - RNA, 23 nt, Homo sapiens
agguugggaucgguugcaaugcu

SEQ ID NO: 162 - RNA, 22 nt, Homo sapiens
uauugcacucgucccggccucc

SEQ ID NO: 163 - RNA, 22 nt, Homo sapiens
aaagugcuguucgugcagguag

SEQ ID NO: 164 - RNA, 23 nt, Homo sapiens
uuuggcacuagcacauuuuugcu

SEQ ID NO: 165 - RNA, 22 nt, Homo sapiens
ugagguaguaaguuguauuguu

SEQ ID NO: 166 - RNA, 22 nt, Homo sapiens
aacccguagauccgaucuugug

SEQ ID NO: 167 - RNA, 22 nt, Homo sapiens
cacccguagaaccgaccuugcg

SEQ ID NO: 168 - RNA, 22 nt, Homo sapiens
aacccguagauccgaacuugug

SEQ ID NO: 169 - RNA, 22 nt, Homo sapiens
uacaguacugugauaacugaag

SEQ ID NO: 170 - RNA, 24 nt, Homo sapiens
caguuaucacagugcugaugcugu

SEQ ID NO: 171 - RNA, 23 nt, Homo sapiens
agcagcauuguacagggcuauga

SEQ ID NO: 172 - RNA, 21 nt, Homo sapiens
uaaagugcugacagugcagau

SEQ ID NO: 173 - RNA, 20 nt, Homo sapiens
ccgcacuguggguacuugcu

SEQ ID NO: 174 - RNA, 23 nt, Homo sapiens
ucccugagacccuuuaaccugug

SEQ ID NO: 175 - RNA, 22 nt, Homo sapiens
acaggugagguucuugggagcc

SEQ ID NO: 176 - RNA, 22 nt, Homo sapiens
ucccugagacccuaacuuguga

SEQ ID NO: 177 - RNA, 22 nt, Homo sapiens
cagugcaauguuaaaagggcau

SEQ ID NO: 178 - RNA, 22 nt, Homo sapiens
cagugcaaugaugaaagggcau

SEQ ID NO: 179 - RNA, 22 nt, Homo sapiens
acucuuucccuguugcacuacu

SEQ ID NO: 180 - RNA, 22 nt, Homo sapiens
uaacagucuacagccauggucg

SEQ ID NO: 181 - RNA, 22 nt, Homo sapiens
uauggcuuuucauuccuaugug

SEQ ID NO: 182 - RNA, 22 nt, Homo sapiens
agcugguguugugaaucaggcc

SEQ ID NO: 183 - RNA, 21 nt, Homo sapiens
accacaggguagaaccacgga

SEQ ID NO: 184 - RNA, 23 nt, Homo sapiens
uaacacugucugguaaagauggc

SEQ ID NO: 185 - RNA, 22 nt, Homo sapiens
ucagugcaucacagaacuuugu

SEQ ID NO: 186 - RNA, 22 nt, Homo sapiens
ucuggcuccgugucuucacucc

SEQ ID NO: 187 - RNA, 21 nt, Homo sapiens
agggagggacgggggcugugc

SEQ ID NO: 188 - RNA, 22 nt, Homo sapiens
acuagacugaagcuccuugagg

SEQ ID NO: 189 - RNA, 22 nt, Homo sapiens
ucgaggagcucacagucuagua

SEQ ID NO: 190 - RNA, 22 nt, Homo sapiens
ucagugcaugacagaacuuggg

SEQ ID NO: 191 - RNA, 22 nt, Homo sapiens
uuaaugcuaaucgugauagggg

SEQ ID NO: 192 - RNA, 23 nt, Homo sapiens
aacauucaacgcugucggugagu

SEQ ID NO: 193 - RNA, 22 nt, Homo sapiens
aacauucauugcugucgguggg

SEQ ID NO: 194 - RNA, 22 nt, Homo sapiens
uuuggcaaugguagaacucaca

SEQ ID NO: 195 - RNA, 23 nt, Homo sapiens
uauggcacugguagaauucacug

SEQ ID NO: 196 - RNA, 23 nt, Homo sapiens
uggagagaaaggcaguuccugau

SEQ ID NO: 197 - RNA, 23 nt, Homo sapiens
caaagaauucuccuuuugggcuu

SEQ ID NO: 198 - RNA, 22 nt, Homo sapiens
caacggaaucccaaaagcagcu

SEQ ID NO: 199 - RNA, 21 nt, Homo sapiens
aacuggccuacaaagucccag

SEQ ID NO: 200 - RNA, 22 nt, Homo sapiens
ugggucuuugcgggcgagauga

SEQ ID NO: 201 - RNA, 24 nt, Homo sapiens
aacuggcccucaaagucccgcuuu

SEQ ID NO: 202 - RNA, 22 nt, Homo sapiens
cgggguuuugagggcgagauga

SEQ ID NO: 203 - RNA, 21 nt, Homo sapiens
uagguaguuucauguuguugg

SEQ ID NO: 204 - RNA, 21 nt, Homo sapiens
uagguaguuuccuguuguugg

SEQ ID NO: 205 - RNA, 22 nt, Homo sapiens
uucaccaccuucuccacccagc

SEQ ID NO: 206 - RNA, 22 nt, Homo sapiens
uaacacugucugguaacgaugu

SEQ ID NO: 207 - RNA, 23 nt, Homo sapiens
uaauacugccugguaaugaugac

SEQ ID NO: 208 - RNA, 22 nt, Homo sapiens
uaauacugccggguaaugaugg

SEQ ID NO: 209 - RNA, 22 nt, Homo sapiens
uccuucauuccaccggagucug

SEQ ID NO: 210 - RNA, 22 nt, Homo sapiens
cugugcgugugacagcggcuga

SEQ ID NO: 211 - RNA, 21 nt, Homo sapiens
uugugcuugaucuaaccaugu

SEQ ID NO: 212 - RNA, 23 nt, Homo sapiens
agcuacauugucugcuggguuuc

SEQ ID NO: 213 - RNA, 24 nt, Homo sapiens
agcuacaucuggcuacugggucuc

SEQ ID NO: 214 - RNA, 24 nt, Homo sapiens
caagucacuagugguuccguuuag

SEQ ID NO: 215 - RNA, 21 nt, Homo sapiens
agggcccccccucaauccugu

SEQ ID NO: 216 - RNA, 23 nt, Homo sapiens
aaaagcuggguugagagggcgaa

SEQ ID NO: 217 - RNA, 23 nt, Homo sapiens
cgcauccccuagggcauuggugu

SEQ ID NO: 218 - RNA, 22 nt, Homo sapiens
ccacugccccaggugcugcugg

SEQ ID NO: 219 - RNA, 19 nt, Homo sapiens
ucucugggccugugucuua

SEQ ID NO: 220 - RNA, 21 nt, Homo sapiens
gccccugggccuauccuagaa

SEQ ID NO: 221 - RNA, 23 nt, Homo sapiens
ucaagagcaauaacgaaaaaugu

SEQ ID NO: 222 - RNA, 20 nt, Homo sapiens
aacaauauccuggugcugag

SEQ ID NO: 223 - RNA, 24 nt, Homo sapiens
ucucacacagaaaucgcacccguc

SEQ ID NO: 224 - RNA, 23 nt, Homo sapiens
aggggugcuaucugugauugagg

SEQ ID NO: 225 - RNA, 22 nt, Homo sapiens
uaaugccccuaaaaauccuuau

SEQ ID NO: 226 - RNA, 22 nt, Homo sapiens
uuauaauacaaccugauaagug

SEQ ID NO: 227 - RNA, 23 nt, Homo sapiens
ugaggggcagagagcgagacuuu

SEQ ID NO: 228 - RNA, 21 nt, Homo sapiens
caaaacgugaggcgcugcuau

SEQ ID NO: 229 - RNA, 22 nt, Homo sapiens
aaugacacgaucacucccguug

SEQ ID NO: 230 - RNA, 22 nt, Homo sapiens
uaauacugucugguaaaaccgu

SEQ ID NO: 231 - RNA, 22 nt, Homo sapiens
uguuugcagaggaaacugagac

SEQ ID NO: 232 - RNA, 22 nt, Homo sapiens
uaugugccuuuggacuacaucg

SEQ ID NO: 233 - RNA, 23 nt, Homo sapiens
augcaguccaugggcauauacac

SEQ ID NO: 234 - RNA, 22 nt, Homo sapiens
ucaggcucaguccccucccgau

SEQ ID NO: 235 - RNA, 23 nt, Homo sapiens
aguggggaacccuuccaugagga

SEQ ID NO: 236 - RNA, 23 nt, Homo sapiens
gucaacacuugcugguuuccucu

SEQ ID NO: 237 - RNA, 23 nt, Homo sapiens
aaaaugguucccuuuagaguguu

SEQ ID NO: 238 - RNA, 22 nt, Homo sapiens
caugccuugaguguaggaccgu

SEQ ID NO: 239 - RNA, 22 nt, Homo sapiens
cggggaucaucaugucacgaga

SEQ ID NO: 240 - RNA, 22 nt, Homo sapiens
cacgcucaugcacacacccaca

SEQ ID NO: 241 - RNA, 21 nt, Homo sapiens
uaacugguugaacaacugaac

SEQ ID NO: 242 - RNA, 21 nt, Homo sapiens
uuaugguuugccugggacuga

SEQ ID NO: 243 - RNA, 22 nt, Homo sapiens
aaauuauuguacaucggaugag

SEQ ID NO: 244 - RNA, 22 nt, Homo sapiens
aaggagcuuacaaucuagcugg

SEQ ID NO: 245 - RNA, 22 nt, Homo sapiens
cggccccacgcaccaggguaag

SEQ ID NO: 246 - RNA, 21 nt, Homo sapiens
cugcccuggcccgagggaccg

SEQ ID NO: 247 - RNA, 22 nt, Homo sapiens
ugagguaguagguuguauaguu

SEQ ID NO: 248 - RNA, 22 nt, Homo sapiens
ugagguaguagguugugugguu

SEQ ID NO: 249 - RNA, 22 nt, Homo sapiens
ugagguaguagguuguaugguu

SEQ ID NO: 250 - RNA, 21 nt, Homo sapiens
agagguaguagguugcauagu

SEQ ID NO: 251 - RNA, 21 nt, Homo sapiens
ugagguaggagguuguauagu

SEQ ID NO: 252 - RNA, 22 nt, Homo sapiens
ugagguaguagauuguauaguu

SEQ ID NO: 253 - RNA, 21 nt, Homo sapiens
ugagguaguaguuuguacagu

SEQ ID NO: 254 - RNA, 21 nt, Homo sapiens
ugagguaguaguuugugcugu

SEQ ID NO: 255 - RNA, 22 nt, Homo sapiens
uagcagcacaucaugguuuaca

SEQ ID NO: 256 - RNA, 22 nt, Homo sapiens
uagcagcacguaaauauuggcg

SEQ ID NO: 257 - RNA, 24 nt, Homo sapiens
caaagugcuuacagugcagguagu

SEQ ID NO: 258 - RNA, 22 nt, Homo sapiens
uaaggugcaucuagugcagaua

SEQ ID NO: 259 - RNA, 23 nt, Homo sapiens
uagcuuaucagacugauguugac

SEQ ID NO: 260 - RNA, 22 nt, Homo sapiens
caacaccagucgaugggcuguc

SEQ ID NO: 261 - RNA, 22 nt, Homo sapiens
aagcugccaguugaagaacugu

SEQ ID NO: 262 - RNA, 22 nt, Homo sapiens
aucacauugccagggauuucca

SEQ ID NO: 263 - RNA, 23 nt, Homo sapiens
aucacauugccagggauuaccac

SEQ ID NO: 264 - RNA, 22 nt, Homo sapiens
cauugcacuugucucggucuga

SEQ ID NO: 265 - RNA, 22 nt, Homo sapiens
uucaaguaauccaggauaggcu

SEQ ID NO: 266 - RNA, 23 nt, Homo sapiens
uucaaguaauucaggauagguug

SEQ ID NO: 267 - RNA, 21 nt, Homo sapiens
uucacaguggcuaaguuccgc

SEQ ID NO: 268 - RNA, 22 nt, Homo sapiens
uucacaguggcuaaguucugca

SEQ ID NO: 269 - RNA, 23 nt, Homo sapiens
uagcaccaucugaaaucgguuau

SEQ ID NO: 270 - RNA, 23 nt, Homo sapiens
uagcaccauuugaaaucaguguu

SEQ ID NO: 271 - RNA, 22 nt, Homo sapiens
cuuucagucggauguuugcagc

SEQ ID NO: 272 - RNA, 21 nt, Homo sapiens
ggcaagaugcuggcauagcug

SEQ ID NO: 273 - RNA, 20 nt, Homo sapiens
gugcauuguaguugcauugc

SEQ ID NO: 274 - RNA, 23 nt, Homo sapiens
uggcagugucuuagcugguuguu

SEQ ID NO: 275 - RNA, 22 nt, Homo sapiens
uauugcacuugucccggccugu

SEQ ID NO: 276 - RNA, 22 nt, Homo sapiens
ugagguaguaaguuguauuguu

SEQ ID NO: 277 - RNA, 22 nt, Homo sapiens
aacccguagauccgaucuugug

SEQ ID NO: 278 - RNA, 22 nt, Homo sapiens
cacccguagaaccgaccuugcg

SEQ ID NO: 279 - RNA, 22 nt, Homo sapiens
aacccguagauccgaacuugug

SEQ ID NO: 280 - RNA, 22 nt, Homo sapiens
uuaaggcacgcggugaaugcca

SEQ ID NO: 281 - RNA, 23 nt, Homo sapiens
ucccugagacccuuuaaccugug

SEQ ID NO: 282 - RNA, 22 nt, Homo sapiens
ucccugagacccuaacuuguga

SEQ ID NO: 283 - RNA, 22 nt, Homo sapiens
ucguaccgugaguaauaaugcg

SEQ ID NO: 284 - RNA, 22 nt, Homo sapiens
ucggauccgucugagcuuggcu

SEQ ID NO: 285 - RNA, 21 nt, Homo sapiens
ucacagugaaccggucucuuu

SEQ ID NO: 286 - RNA, 22 nt, Homo sapiens
uugguccccuucaaccagcugu

SEQ ID NO: 287 - RNA, 21 nt, Homo sapiens
uaccacaggguagaaccacgg

SEQ ID NO: 288 - RNA, 23 nt, Homo sapiens
uguaguguuuccuacuuuaugga

SEQ ID NO: 289 - RNA, 22 nt, Homo sapiens
ugagaugaagcacuguagcuca

SEQ ID NO: 290 - RNA, 23 nt, Homo sapiens
guccaguuuucccaggaaucccu

SEQ ID NO: 291 - RNA, 22 nt, Homo sapiens
ugagaacugaauuccauggguu

SEQ ID NO: 292 - RNA, 22 nt, Homo sapiens
ucagugcaucacagaacuuugu

SEQ ID NO: 293 - RNA, 22 nt, Homo sapiens
ucgaggagcucacagucuagua

SEQ ID NO: 294 - RNA, 22 nt, Homo sapiens
acuagacugaagcuccuugagg

SEQ ID NO: 295 - RNA, 22 nt, Homo sapiens
uagguuauccguguugccuucg

SEQ ID NO: 296 - RNA, 22 nt, Homo sapiens
uuaaugcuaaucgugauagggg

SEQ ID NO: 297 - RNA, 22 nt, Homo sapiens
accaucgaccguugauuguacc

SEQ ID NO: 298 - RNA, 22 nt, Homo sapiens
aacauucauugcugucgguggg

SEQ ID NO: 299 - RNA, 22 nt, Homo sapiens
aacauucauuguugucgguggg

SEQ ID NO: 300 - RNA, 22 nt, Homo sapiens
uuuggcaaugguagaacucaca

SEQ ID NO: 301 - RNA, 23 nt, Homo sapiens
uggagagaaaggcaguuccugau

SEQ ID NO: 302 - RNA, 22 nt, Homo sapiens
caacggaaucccaaaagcagcu

SEQ ID NO: 303 - RNA, 21 nt, Homo sapiens
uagguaguuuccuguuguugg

SEQ ID NO: 304 - RNA, 23 nt, Homo sapiens
cccaguguucagacuaccuguuc

SEQ ID NO: 305 - RNA, 24 nt, Homo sapiens
cccaguguuuagacuaucuguuca

SEQ ID NO: 306 - RNA, 22 nt, Homo sapiens
uaacacugucugguaacgaugu

SEQ ID NO: 307 - RNA, 23 nt, Homo sapiens
uaauacugccugguaaugaugac

SEQ ID NO: 308 - RNA, 22 nt, Homo sapiens
uaauacugccggguaaugaugg

SEQ ID NO: 309 - RNA, 22 nt, Homo sapiens
uccuucauuccaccggagucug

SEQ ID NO: 310 - RNA, 22 nt, Homo sapiens
cugugcgugugacagcggcuga

SEQ ID NO: 311 - RNA, 20 nt, Homo sapiens
acagcaggcacagacaggca

SEQ ID NO: 312 - RNA, 21 nt, Homo sapiens
ugauuguccaaacgcaauucu

SEQ ID NO: 313 - RNA, 24 nt, Homo sapiens
agcuacaucuggcuacugggucuc

SEQ ID NO: 314 - RNA, 21 nt, Homo sapiens
uaugugggaugguaaaccgcu

SEQ ID NO: 315 - RNA, 23 nt, Homo sapiens
aaaagcuggguugagagggcgaa

SEQ ID NO: 316 - RNA, 23 nt, Homo sapiens
cgcauccccuagggcauuggugu

SEQ ID NO: 317 - RNA, 22 nt, Homo sapiens
cuggcccucucugcccuuccgu

SEQ ID NO: 318 - RNA, 22 nt, Homo sapiens
uaaugccccuaaaaauccuuau

SEQ ID NO: 319 - RNA, 21 nt, Homo sapiens
aacauagaggaaauuccacgu

SEQ ID NO: 320 - RNA, 22 nt, Homo sapiens
uuauaauacaaccugauaagug

SEQ ID NO: 321 - RNA, 22 nt, Homo sapiens
uuuguucguucggcucgcguga

SEQ ID NO: 322 - RNA, 21 nt, Homo sapiens
aucauagaggaaaauccacgu

SEQ ID NO: 323 - RNA, 22 nt, Homo sapiens
uauacaagggcaagcucucugu

SEQ ID NO: 324 - RNA, 23 nt, Homo sapiens
ugaggggcagagagcgagacuuu

SEQ ID NO: 325 - RNA, 21 nt, Homo sapiens
cagcagcaauucauguuuuga

SEQ ID NO: 326 - RNA, 21 nt, Homo sapiens
caaaacgugaggcgcugcuau

SEQ ID NO: 327 - RNA, 22 nt, Homo sapiens
aaugacacgaucacucccguug

SEQ ID NO: 328 - RNA, 20 nt, Homo sapiens
uuuugcgauguguuccuaau

SEQ ID NO: 329 - RNA, 23 nt, Homo sapiens
augcaguccaugggcauauacac

SEQ ID NO: 330 - RNA, 23 nt, Homo sapiens
ugaaacauacacgggaaaccucu

SEQ ID NO: 331 - RNA, 22 nt, Homo sapiens
aaacaaacauggugcacuucuu

SEQ ID NO: 332 - RNA, 23 nt, Homo sapiens
gucaacacuugcugguuuccucu

SEQ ID NO: 333 - RNA, 20 nt, Homo sapiens
ugugacagauugauaacuga

SEQ ID NO: 334 - RNA, 22 nt, Homo sapiens
cacgcucaugcacacacccaca

(19) United States
(12) Patent Application Publication
(10) Pub. No.: US 2010/0234445 A1
(43) Pub. Date: Sep. 16, 2010

(54) Patterns of known and novel small RNAS in human cervical cancer

(76) Inventors: Weng Onn Lui, Stanford, CA (US); Nader Pourmand, San Mateo, CA (US); Bruce K. Patterson, Palo Alto, CA (US); Andrew Fire, Stanford, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/459,409

(22) Filed: Jun. 30, 2009

Related U.S. Application Data
(60) Provisional application No. 61/133,734, filed on Jul. 1, 2008.

(51) Int. Cl. A61K 31/713; C12Q 1/68; C40B 30/00; A61P 35/00

(52) U.S. Cl. 514/44 A; 435/6; 506/7

(57) ABSTRACT

Small RNA sequences that are differentially expressed in SCCC cells are provided. The sequences find use in diagnosis of cancer, and classification of cancer cells according to expression profiles. The methods are useful for detecting cervical cancer cells, facilitating diagnosis of cervical cancer and the severity of the cancer (e.g., tumor grade, tumor burden, and the like) in a subject, facilitating a determination of the prognosis of a subject, and assessing the responsiveness of the subject to therapy.

### GOVERNMENT RIGHTS

This invention was made with Government support under contract HG000205 awarded by the National Institutes of Health. The Government has certain rights in this invention.

### BACKGROUND OF THE INVENTION

Cervical cancer is the second most common cancer diagnosis in women and is linked to high-risk human papillomavirus infection 99.7% of the time. According to the American Cancer Society, every year over 12,000 new cases of invasive cervical cancer are diagnosed and more than 4,000 women die of the disease. Furthermore, there are approximately 400,000 cases of cervical cancer and close to 200,000 deaths annually worldwide. Human papillomaviruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

Squamous cell carcinoma of the cervix (SCCC) is by far the most common histological type of cervical cancer. The Pap test, based upon cytological examination of vaginal exfoliated cells, has reduced the incidence and mortality of cervical cancer by 60-70% where it has been used in routine screening programs. However, where no Pap screening programs are in place or where a population does not participate in screening programs, the incidence and mortality of the disease remains high.

A limitation of the Pap test is that it is morphologically based, and the accuracy can be problematic because of pre-analytical processing and interpretive errors. There is inter-observer variation in the reading and classifying of the cytological smears. Molecular-based testing for high-risk human papillomavirus (HPV) strains is mostly performed when Pap tests are inconclusive and is generally used in conjunction with liquid based cytological methods. These tests are still being investigated in large studies to further determine their usefulness.

Current guidelines for managing patients with atypical squamous cells call for assigning these cases into Pap subcategories that distinguish the cases that have a high risk for invasive carcinoma (ASC-H) (HSIL) from the cases of undetermined significance (ASC-US). A molecular test based upon multiple diagnostic markers that are associated with the cancer phenotype potentially could identify SCCC with higher specificity than currently available tests. Furthermore, the identification of a subset of those expressed in SCCC would be helpful in subcategory assignment.

Identification of expressed sequence that are differentially expressed in cancerous, pre-cancerous, or low metastatic potential cells relative to normal cells of the same tissue type, provides the basis for diagnostic tools, facilitates drug discovery by providing for targets for candidate agents, and further serves to identify therapeutic targets for cancer therapies that are more tailored for the type of cancer to be treated. Early disease diagnosis is of central importance to halting disease progression, and reducing morbidity. The product of a differentially expressed sequence can be the basis for screening assays to identify chemotherapeutic agents that modulate its activity (e.g. its expression, biological activity, and the like)

Analysis of a patient sample to identify the sequences that are differentially expressed, and administration of therapeutic agent(s) designed to modulate the activity of those differentially expressed sequences, provides the basis for more specific, rational cancer therapy that may result in diminished adverse side effects relative to conventional therapies. Furthermore, confirmation that a tumor poses less risk to the patient (e.g., that the tumor is benign) can avoid unnecessary therapies. In short, identification of expressed sequences that are differentially expressed in cancerous cells can provide the basis of therapeutics, diagnostics, prognostics, therametrics, and the like.

MicroRNAs (miRNAs) are single-stranded RNAs of ˜22 nucleotides in length that are generated by the RNase-III type enzyme Dicer from endogeneous hairpin-shaped transcripts. miRNAs exert at least a part of their biological effects as guides for post-transcriptional gene silencing, producing sequence-specific mRNA cleavage or translational repression that can have dramatic effects on cellular phenotype.

To date, there are few miRNAs whose physiological function has been elucidated in vivo and whose targets are known. Studies in model organisms have revealed that miRNAs are involved in the control of developmental timing, cell proliferation, neuronal cell fate, apoptosis, morphogenesis, fat metabolism and tumorigenesis, as well as in a variety of patterning processes in plants. In mammals, miRNAs have been shown to regulate B-cell differentiation, adipocyte differentiation, insulin secretion, antiviral defense, cardiogenesis and tumorigenesis.

Accumulating evidence demonstrates that changes in miRNA levels may accompany disregulated growth and apoptosis in some cancers. Reductions in expression of miR-15a and miR-16, let-7a, or miR-143 and miR-145 have been reported in chronic lymphocytic leukemia, lung cancer, and colorectal neoplasia, respectively. On the other hand, significantly higher levels of miR-155 are present in diffuse large B-cell lymphoma with activated B-cell phenotype. Recently, several groups demonstrated that overexpression of the miR17-92 cluster in mice predisposed to tumor formation and can accelerate progression of tumorigenesis.

In addition to indications of potential roles in tumorigenesis, a number of recent studies have pioneered the use of miRNA expression profiles in tumor classification. Strikingly, in one of these studies, a profile comprising 217 previously characterized miRNAs appeared to be more effective in cancer classification, particularly in poorly differentiated tumors, than mRNA microarray profiles containing ˜16,000 protein-coding genes (Lu et al. Nature 2005; 435:834-8). In addition, accumulating evidence demonstrate the prognostic potential of miRNA expression profiles in several cancer types (Calin et al. N Engl J Med 2005; 353:1793-801; Yanaihara et al. Cancer Cell 2006; 9:189-98; Roldo et al. J Clin Oncol 2006; 24:4677-84).

Full application of research and clinical tools that derive from miRNA expression profiling will depend on a complete picture of small RNA populations associated with tumors. The present invention identifies sequences, including novel miRNAs, differentially expressed in cervical carcinoma. The identification of these genes provides insight into the understanding of the biology of SCCC, and the sequences identified could have use in diagnosis and treatment.

### SUMMARY OF THE INVENTION

The present invention provides methods and compositions useful in detection of cervical cancer cells, identification of agents that modulate the phenotype of cervical cancer, and identification of therapeutic targets for chemotherapy. More specifically, the invention provides polynucleotide sequences, including known and novel miRNA sequences that are differentially expressed in cervical cancer cells, particularly squamous cell carcinoma of the cervix (SCCC). These polynucleotides make possible a variety of diagnostic, therapeutic, and drug discovery methods. In some embodiments, a polynucleotide that is differentially expressed in SCCC is used in diagnostic assays to detect cervical cancer. In other embodiments, a polynucleotide that is differentially expressed in SCCC is itself a target for therapeutic intervention.

In one embodiment of the invention, the invention provides a method for detecting or assessing SCCC. The method involves identifying populations of small RNAs present in tissue from the patient or contacting a test sample obtained from a tissue that is suspected of comprising cervical cancer cells with a probe for detecting a RNA sequence differentially expressed in SCCC. Such methods may detect one, two, three four, five, six or more RNA species, and up to 20, up to 30, up to 40, up to 50 or more RNA species. Many embodiments of the invention involve a sequence identifiable or comprising a sequence selected from the sequences presented in Tables 1, 2 and 3.

In some embodiments of the invention, the differentially expressed sequence is selected from the novel miRNA sequences set forth in Table 1, e.g. miR374b; miR-933; miR-769-3p; miR671; miR-934; miR-935; miR-936; miR-937; miR-938; miR-939; miR-940; miR-941; miR-942; miR-943; miR-944; miR-708; miR-874-5p; and miR-874-3p. These small RNA segments were designated as novel candidate miRNAs, based on a predicted hairpin precursor with the following properties: (1) complete containment of the cDNA sequence within one arm of a hairpin, (2) at least 16 nucleotides of the cDNA sequence involved in base-pairing, and (3) identification as the lowest free energy structure by mfold.

In some embodiments of the invention, the differentially expressed sequence is selected from the small RNA sequences with non-canonical hairpins set forth in Table 2, e.g. sRNA-cer1; sRNA-cer2; sRNA-cer3; sRNA-cer4; sRNA-cer5; sRNA-cer6; sRNA-cer7; sRNA-cer8 and sRNA-cer9.

In some embodiments of the invention, the differentially expressed sequence is selected from the novel small RNAs without significant hairpins set forth in Table 3, e.g. sRNA-1; sRNA-2; sRNA-3; sRNA-4; sRNA-5; sRNA-6; sRNA-7; sRNA-8; sRNA-9; sRNA-10; sRNA-11; sRNA-12; sRNA-13; sRNA-14; sRNA-15; sRNA-16; sRNA-17; sRNA-18; sRNA-19; sRNA-20; sRNA-21; sRNA-22; sRNA-23; sRNA-24; sRNA-25; sRNA-26; sRNA-27; sRNA-28; sRNA-29; sRNA-30; sRNA-31; sRNA-32; and sRNA-33.

In some embodiments of the invention, the differentially expressed sequence is identified by the method of ligation capture, sequencing, and RNA analysis used to discover the RNAs set forth in Tables 1, 2, and 3.

In one preferred embodiment, the differentially expressed sequence is one or a combination of the miRNA sequences let-7b, let-7c, miR-23b, miR196b, miR-143 and miR-21, where let-7b, let-7c, miR-23b, miR196b, and miR-143 have significantly reduced expression in cervical cancer and miR-21 has significantly increased expression in cervical cancer. In some embodiments, detection of miR-21 expression is coupled with detection of down-regulation of one or more, two or more, three or more, four, or five of the down-regulated sequences.

In another embodiment of the invention, methods are provided for suppressing or inhibiting a cancerous phenotype of a cervical carcinoma cell, the method comprising introducing into a mammalian cell an expression modulatory agent (e.g. an antisense molecule, inhibitory RNA molecule, RNA-binding molecule, etc.) to inhibit of expression of a sequence identified as upregulated in cervical cancer, e.g. miR-21. Inhibition of expression would be useful as a therapeutic approach if it inhibits development of a cancerous phenotype in the cell. In specific embodiments, the cancerous phenotype is metastasis, aberrant cellular proliferation relative to a normal cell, or loss of contact inhibition of cell growth.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Illustration of miRNA cloning incidence in six cervical cancer cell lines and five normal cervical samples. For the display to reflect relative expression levels, we carried out a normalization using the formula (X− X̅)/ X̅ where, X is the incidence frequency of miRNA in a given sample and X̅ is the mean incidence frequency among all samples. High expression is shown in yellow and low expression in blue. Note that only miRNAs with more than 1% in any samples analyzed are shown. NC=normal cervix.

FIG. 2: Representative miRNAs with significant expression variations between normal cervix and cervical cancer cell lines identified by cloning experiments. The expression was calculated by the fraction of clone number from each pool of library, and represented in percentage. The figure insert shows the mean fraction of clone number from the miRNA pool for each sample group (N, normal cervix; C, cervical cancer cell lines). P-values<0.0001 were considered as significant, determined by χ²-test. Notably, the expression of miR-143, let-7c and miR-196b in normal cervix is significantly lower than that in cervical cancer cell lines, while the expression of miR-21 is significantly higher in cervical cancer cells.

FIG. 3: Northern blot analysis of miR-21 expression and miR-143. A, mature miR-21, detected in all cervical cancer cell lines, was barely detected in many of the normal cervical samples. Substantial higher relative expression of miR-21 in tumor was evident in 72% of the cases. B, mature miR-143 was detected in most of the normal cervical samples, but absent or barely detected in all cervical cancer cell lines and in 16/27 (59%) of the cancer samples. In five cases, the expression of miR-143 was relatively lower in cancer than that in their matched normal cervical samples, while two cases showed relatively more abundance in the tumor samples. Ethidium-bromide-stained rRNA bands are shown as loading controls.

FIG. 4: comparison of small RNA species.

### DETAILED DESCRIPTION OF THE INVENTION

The present invention identifies polynucleotides that are differentially expressed in SCCC cells. Methods are provided in which these polynucleotides are used for detecting, assessing, and reducing the growth of cancer cells. The invention finds use in the prevention, treatment, detection or research of cervical cancer.

The present invention provides methods of using the polynucleotides described herein in diagnosis of cancer, and classification of cancer cells according to expression profiles. The methods are useful for detecting cervical cancer cells, facilitating diagnosis of cervical cancer and the severity of the cancer (e.g., tumor grade, tumor burden, and the like) in a subject, facilitating a determination of the prognosis of a subject, and assessing the responsiveness of the subject to therapy. The detection methods of the invention can be conducted in vitro or in vivo, on isolated cells, or in whole tissues or a bodily fluid, e.g., blood, plasma, serum, urine, and the like. Samples of particular interest include cervical tissue, which may be obtained by biopsy, scrape, swab, and the like.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy, eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998). Reagents, cloning vectors, and kits for genetic manipulation referred to in this disclosure are available from commercial vendors such as BioRad, Stratagene, Invitrogen, Sigma-Aldrich, and ClonTech.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

Cervical cancer is essentially a sexually transmitted disease (see, for example, Bosch and de Sanjosé (2007) Dis Markers. 2007; 23(4):213-27; and Adams et al. (2007) Vaccine 25(16):3007-13. Risk is inversely related to age at first intercourse and directly related to the lifetime number of sexual partners. Risk is also increased for sexual partners of men whose previous partners had cervical cancer. Human papillomavirus (HPV) infection and the development of cervical neoplasia are strongly associated. HPV infection is linked to all grades of cervical intraepithelial neoplasia (CIN) and invasive cervical cancer. Infection with HPV types 16, 18, 31, 33, 35, and 39 increases the risk of neoplasia. However, other factors appear to contribute to malignant transformation. For example, cigarette smoking is associated with an increased risk of CIN and cervical cancer.

Squamous cell carcinoma accounts for 80 to 85% of all cervical cancers. Precursor cells (cervical dysplasia, CIN) develop into invasive cervical cancer over a number of years. CIN grades I, II, and III correspond to mild, moderate, and severe cervical dysplasia. CIN III, which includes severe dysplasia and carcinoma in situ, is unlikely to regress spontaneously and, if untreated, may eventually penetrate the basement membrane, becoming invasive carcinoma. Invasive cervical cancer usually spreads by direct extension into surrounding tissues and the vagina or via the lymphatics to the pelvic and para-aortic lymph nodes drained by the cervix. Hematologic spread is possible.

More than 90% of early asymptomatic cases of CIN can be detected preclinically by cytologic examination of Pap smears obtained directly from the cervix. However, the false-negative rate is 15 to 40%, depending on the patient population and the laboratory. About 50% of patients with cervical cancer have never had a Pap smear or have not had one for >=10 yr. The patients at higher risk for cervical neoplasia are the least likely to be tested regularly. An abnormal Pap smear, i.e. suggesting neoplasia, including dysplasia, CIN, carcinoma in situ, microinvasive carcinoma, or invasive carcinoma, requires further evaluation based on the descriptive diagnosis of the Pap smear and the patient's risk factors.

Suspicious cervical lesions should be biopsied directly. If there is no obvious invasive lesion, colposcopy can be used to identify areas that require biopsy and to localize the lesion. Colposcopy results can be clinically correlated (by assessing characteristic color changes, vascular patterns, and margins) with the results of the Pap smear. If cervical disease is invasive, staging is performed on the basis of the physical examination, with a metastatic survey including cystoscopy, sigmoidoscopy, IV pyelography, chest x-ray, and skeletal x-rays. For early-stage disease (IB or less), chest x-ray is usually the only adjunctive test needed. CT or MRI of the abdomen and pelvis is optional; the results cannot be used to determine the clinical stage.

Invasive squamous cell carcinoma usually remains localized or regional for a considerable time; distant metastases occur late. The 5-yr survival rates are 80 to 90% for stage I, 50 to 65% for stage II, 25 to 35% for stage III, and 0 to 15% for stage IV. Nearly 80% of recurrences manifest within 2 yr. Adverse prognostic factors include lymph node involvement, large tumor size and volume, deep cervical stromal invasion, parametrial invasion, vascular space invasion, and neuroendocrine histology.

As used herein, the terms “a sequence that is differentially expressed in a cancer cell,” and “a polynucleotide that is differentially expressed in a cancer cell” are used interchangeably herein, and generally refer to a polynucleotide, typically a small RNA, that represents or corresponds to a sequence that is differentially expressed in a cancerous cell when compared with a cell of the same cell type that is not cancerous, e.g., RNA is found at levels at least about 25%, at least about 50% to about 75%, at least about 90%, at least about 1.5-fold, at least about 2-fold, at least about 3-fold, at least about 5-fold, at least about 10-fold, or at least about 50-fold or more, different (e.g., higher or lower). The comparison can be made in tissue, for example, if one is using in situ hybridization or another assay method that allows some degree of discrimination among cell types in the tissue. The comparison may also or alternatively be made between cells removed from their tissue source analyzed either as groups of cells or individual cells. The term “a polypeptide associated with cancer” refers to a polypeptide encoded by a polynucleotide that is differentially expressed in a cancer cell.

The polynucleotide may correspond to a sequence that is over-expressed, under-expressed, or that is modified in a cancerous cell relative to a normal cell. The sequence in the cancerous cell may contain a deletion, insertion, substitution, or translocation.

“Diagnosis” as used herein generally includes determination of a subject's susceptibility to a disease or disorder, determination as to whether a subject is presently affected by a disease or disorder, prognosis of a subject affected by a disease or disorder (e.g., identification of pre-metastatic or metastatic cancerous states, stages of cancer, or responsiveness of cancer to therapy), and use of therametrics (e.g., monitoring a subject's condition to provide information as to the effect or efficacy of therapy).

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

“Therapeutic target” refers to a gene or gene product that, upon modulation of its activity (e.g., by modulation of expression, biological activity, and the like), can provide for modulation of the cancerous phenotype.

As used throughout, “modulation” is meant to refer to an increase or a decrease in the indicated phenomenon (e.g., modulation of a biological activity refers to an increase in a biological activity or a decrease in a biological activity).

MicroRNAs (miRNAs) are an abundant class of non-coding RNAs that are believed to be important in many biological processes through regulation of gene expression. These noncoding RNAs can play important roles in development by targeting the messages of protein-coding genes for cleavage or repression of productive translation. Humans have hundreds or thousands of genes that encode miRNAs.

miRNAs are single stranded RNA molecules that range in length from about 20 to about 25 nt, such as from about 21 to about 24 nt, e.g., 22 or 23 nt. In some embodiments of the invention, polynucleotide agents are introduced to modulate miRNA expression or activity. The miRNA agent may increase or decrease the level or activity of a targeted miRNA in the targeted cell. Where the agent is an inhibitory agent, it may inhibit the activity of the target miRNA by reducing the amount of targeted miRNA present in the targeted cells, where the target cell may be present in vitro or in vivo. By “reducing the amount of” is meant that the level or quantity of the target miRNA in the target cell is reduced by at least about 1.5-fold, usually by at least about 2-fold, e.g., 5-fold, 10-fold, 15-fold, 20-fold, 50-fold, 100-fold or more, as compared to a control, i.e., an identical target cell not treated according to the subject methods.

Where the agent increases the activity of the targeted miRNA in a cell, the amount of targeted miRNA is increased in the targeted cells, where the target cell may be present in vitro or in vivo. By “increasing the amount of” is meant that the level or quantity of the target miRNA in the target cell is increased by at least about 1.5-fold, usually by at least about 2-fold, e.g., 5-fold, 10-fold, 15-fold, 20-fold, 50-fold, 100-fold or more, as compared to a control, i.e., an identical target cell not treated according to the subject methods.

By miRNA inhibitory agent is meant an agent that inhibits the activity of the target miRNA. The inhibitory agent may inhibit the activity of the target miRNA by a variety of different mechanisms. In certain embodiments, the inhibitory agent is one that binds to the target miRNA and, in doing so, inhibits its activity. Representative miRNA inhibitory agents include, but are not limited to: antisense oligonucleotides, and the like. Other agents of interest include, but are not limited to: naturally occurring or synthetic small molecule compounds of interest, which include numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing appropriate screening protocols.

A target miRNA may or may not be completely complementary to the introduced miRNA agent. If not completely complementary, the miRNA and its corresponding agent are at least substantially complementary, such that the amount of mismatches present over the length of the miRNA, (ranging from about 20 to about 25 nt) will not exceed about 8 nt, and will in certain embodiments not exceed about 6 or 5 nt, e.g., 4 nt, 3 nt, 2 nt or 1 nt.

The antisense reagent may be antisense oligonucleotides (ODN), particularly synthetic ODN having chemical modifications from native nucleic acids, or nucleic acid constructs that express such antisense molecules as RNA. The antisense sequence is complementary to the targeted miRNA, and inhibits its expression. One or a combination of antisense molecules may be administered, where a combination may comprise multiple different sequences.

Antisense molecules may be produced by expression of all or a part of the target miRNA sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25, usually not more than about 23-22 nucleotides in length, where the length is governed by efficiency of inhibition, specificity, including absence of cross-reactivity, and the like.

Antisense oligonucleotides may be chemically synthesized by methods known in the art (see Wagner et al. (1993) supra. and Milligan et al., supra.) Preferred oligonucleotides are chemically modified from the native phosphodiester structure, in order to increase their intracellular stability and binding affinity. A number of such modifications have been described in the literature that alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The alpha.-anomer of deoxyribose may be used, where the base is inverted with respect to the natural.beta.-anomer. The 2′-OH of the ribose sugar may be altered to form 2′-O-methyl, 2′-Fluoro, or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Anti-sense molecules of interest include antagomir RNAs, e.g. as described by Krutzfeldt et al., supra., herein specifically incorporated by reference. Small interfering double-stranded RNAs (siRNAs) engineered with certain ‘drug-like’ properties such as chemical modifications for stability and cholesterol conjugation for delivery have been shown to achieve therapeutic silencing of an endogenous gene in vivo. To develop a pharmacological approach for silencing miRNAs in vivo, chemically modified, cholesterol-conjugated single-stranded RNA analogues complementary to miRNAs were developed, termed ‘antagomirs’. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. In representative embodiments, the RNAi agent targets the precursor molecule of the microRNA, known as pre-microRNA molecule. By RNAi agent is meant an agent that modulates expression of microRNA by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an siRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNA can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

Where it is desirable to increase a target miRNA expression in a cell, an agent may be the targeted miRNA itself, including any of the modified oligonucleotides described above with respect to antisense, e.g. cholesterol conjugates, phosphorothioates linkages, and the like. Alternatively, a vector that expresses the targeted miRNA, including the pre-miRNA sequence relevant to the targeted organism, may be used.

Expression vectors may be used to introduce the target gene into a cell. Such vectors generally have convenient restriction sites located near the promoter sequence to provide for the insertion of nucleic acid sequences. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to transiently or stably be maintained in the cells, usually for a period of at least about one day, more usually for a period of at least about several days to several weeks.

The expression cassette may employ an exogenous transcriptional initiation region, i.e. a promoter other than the native promoter. The promoter is functional in host cells, particularly host cells targeted by the cassette. The promoter may be introduced by recombinant methods in vitro, or as the result of homologous integration of the sequence by a suitable host cell. The promoter is operably linked to the miRNA sequence. Expression vectors conveniently will have restriction sites located near the promoter sequence to facilitate the insertion of miRNA sequences. The expression cassettes may be introduced into a variety of vectors. Promoters of interest may be inducible or constitutive, usually constitutive, and will provide for high levels of transcription in the vaccine recipient cells. The promoter may be active only in the recipient cell type, or may be broadly active in many different cell types. Many strong promoters for mammalian cells are known in the art, including the .beta.-actin promoter, SV40 early and late promoters, immunoglobulin promoter, human cytomegalovirus promoter, retroviral LTRs, etc. The promoters may or may not be associated with enhancers, where the enhancers may be naturally associated with the particular promoter or associated with a different promoter.

A termination region may be provided 3′ to the coding region, where the termination region may be naturally associated with the variable region domain or may be derived from a different source. A wide variety of termination regions may be employed without adversely affecting expression.

The various manipulations may be carried out in vitro or may be performed in an appropriate host, e.g. E. coli. After each manipulation, the resulting construct may be cloned, the vector isolated, and the DNA screened or sequenced to ensure the correctness of the construct. The sequence may be screened by restriction analysis, sequencing, or the like.

As indicated above, the miRNA agent can be introduced into the target cell(s) using any convenient protocol, where the protocol will vary depending on whether the target cells are in vitro or in vivo. A number of options can be utilized to deliver the dsRNA into a cell or population of cells such as in a cell culture, tissue, organ or embryo. For instance, RNA can be directly introduced intracellularly. Various physical methods are generally utilized in such instances, such as administration by microinjection (see, e.g., Zernicka-Goetz, et al. (1997) Development 124:1133-1137; and Wianny, et al. (1998) Chromosoma 107: 430-439). Other options for cellular delivery include permeabilizing the cell membrane and electroporation in the presence of the dsRNA, liposome-mediated transfection, or transfection using chemicals such as calcium phosphate. A number of established gene therapy techniques can also be utilized to introduce the dsRNA into a cell. By introducing a viral construct within a viral particle, for instance, one can achieve efficient introduction of an expression construct into the cell and transcription of the RNA encoded by the construct.

For example, the inhibitory agent can be fed directly to, injected into, the host organism containing the target gene. The agent may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

In certain embodiments, a hydrodynamic nucleic acid administration protocol is employed. Where the agent is a ribonucleic acid, the hydrodynamic ribonucleic acid administration protocol described in detail below is of particular interest. Where the agent is a deoxyribonucleic acid, the hydrodynamic deoxyribonucleic acid administration protocols described in Chang et al., J. Virol. (2001) 75:3469-3473; Liu et al., Gene Ther. (1999) 6:1258-1266; Wolff et al., Science (1990) 247: 1465-1468; Zhang et al., Hum. Gene Ther. (1999) 10:1735-1737: and Zhang et al., Gene Ther. (1999) 7:1344-1349; are of interest.

Additional nucleic acid delivery protocols of interest include, but are not limited to: those described in U.S. patents of interest include U.S. Pat. No. 5,985,847 and U.S. Pat. No. 5,922,687 (the disclosures of which are herein incorporated by reference); WO/11092; Acsadi et al., New Biol. (1991) 3:71-81; Hickman et al., Hum. Gen. Ther. (1994) 5:1477-1483; and Wolff et al., Science (1990) 247: 1465-1468; etc.

Depending n the nature of the agent, the active agent(s) may be administered to the host using any convenient means capable of resulting in the desired modulation of miRNA expression in the target cell. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc., administration.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill in the art will readily appreciate that dose levels can vary as a function of the specific compound, the nature of the delivery vehicle, and the like. Preferred dosages for a given compound are readily determinable by those of skill in the art by a variety of means.

The invention provides polynucleotides that represent small RNA sequences that are expressed in human SCCC. These small RNA sequences have uses that include, but are not limited to, diagnostic probes and primers as starting materials for probes and primers, as discussed herein. Nucleic acid compositions include fragments and primers, and are at least about 15 by in length, at least about 20, at least about 25, and usually not more than about 30 by in length. Also included are variants or degenerate variants of a sequence provided herein. In general, a variants of a polynucleotide provided herein have a fragment of sequence identity that is greater than at least about 85%, or greater than at least about 90%, 95%, 96%, 97%, 98%, 99% or more (i.e. 100%) as compared to an identically sized fragment of a provided sequence as determined by the Smith-Waterman homology search algorithm as implemented in MPSRCH program (Oxford Molecular). Nucleic acids having sequence similarity can be detected by hybridization under low stringency conditions. Sequence identity can be determined by hybridization under high stringency conditions. Hybridization methods and conditions are well known in the art, see, e.g., U.S. Pat. No. 5,707,829. Nucleic acids that are substantially identical to the provided polynucleotide sequences, e.g. allelic variants, genetically altered versions of the small RNA sequences, etc., bind to the provided polynucleotide sequences under stringent hybridization conditions, suitably corrected for short sequences.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.)

Probes specific to the small RNA sequences described herein can be generated using the polynucleotide sequences disclosed herein. The probes may be a fragment of a polynucleotide sequences provided herein, or more usually, the complete small RNA sequence. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein.

The probes described herein can be used to, for example, determine the presence or absence of any one of the polynucleotide provided herein or variants thereof in a sample. These and other uses are described in more detail below. In one embodiment, the probes are used in an RDA method for analysis of gene expression. In another embodiment, real time PCR analysis is used to analyze gene expression. In other embodiments the probes are utilized in an array format to determine expression levels.

### Diagnostic Methods

The present invention provides methods of using the polynucleotides described herein in diagnosis of cancer, and classification of cancer cells according to expression profiles. The methods are useful for detecting cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of one or more small RNA sequences differentially expressed in a cancer cell. The detection methods of the invention can be conducted in vitro or in vivo, on isolated cells, or in whole tissues or a bodily fluid, e.g., blood, plasma, serum, urine, and the like).

In general, methods of the invention involving detection of a gene product (e.g., RNA) by direct sequencing of small RNA molecules or by contacting a sample with a probe specific for the gene product of interest. “Probe” as used herein in such methods is meant to refer to a molecule that specifically binds a gene product of interest (e.g., the probe binds to the target gene product with a specificity sufficient to distinguish binding to target over non-specific binding to non-target (background) molecules). “Probes” include, but are not necessarily limited to, nucleic acid probes e.g., DNA, RNA, modified nucleic acid, and the like.

For hybridization-based approaches, the probe and sample suspected of having the gene product of interest are contacted under conditions suitable for binding of the probe to the gene product. For example, contacting is generally for a time sufficient to allow binding of the probe to the gene product (e.g., from several minutes to a few hours), and at a temperature and conditions of osmolarity and the like that provide for binding of the probe to the gene product at a level that is sufficiently distinguishable from background binding of the probe (e.g., under conditions that minimize non-specific binding). Suitable conditions for probe-target gene product binding can be readily determined using controls and other techniques available and known to one of ordinary skill in the art.

In some embodiments, methods are provided for detecting a cancer cell by detecting expression in the cell of a transcript or that is differentially expressed in a cancer cell. Any of a variety of known methods can be used for detection, including, but not limited to, direct sequencing, detection of a transcript by hybridization with a polynucleotide that hybridizes to a polynucleotide that is differentially expressed in a cancer cell; detection of a transcript by a polymerase chain reaction using specific oligonucleotide primers; in situ hybridization of a cell using as a probe a polynucleotide that hybridizes to a gene that is differentially expressed in a cancer cell and the like.

In many embodiments, the levels of a subject gene product are measured. By measured is meant qualitatively or quantitatively estimating the level of the gene product in a first biological sample either directly (e.g. by determining or estimating absolute levels of gene product) or relatively by comparing the levels to a second control biological sample. In many embodiments the second control biological sample is obtained from an individual not having not having cancer. As will be appreciated in the art, once a standard control level of gene expression is known, it can be used repeatedly as a standard for comparison. Other control samples include samples of cancerous tissue.

The methods can be used to detect and/or measure RNA levels of a gene that is differentially expressed in a cancer cell. In some embodiments, the methods comprise: contacting a sample with a polynucleotide that corresponds to a differentially expressed gene described herein under conditions that allow hybridization; and detecting hybridization, if any. Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell. Conditions that allow hybridization are known in the art, and have been described in more detail above.

Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide, and direct sequencing of RNA or cDNA produced from RNA. For probe-based approaches, a variety of labels and labeling methods for polynucleotides are known in the art and can be used in the assay methods of the invention. Specific hybridization can be determined by comparison to appropriate controls.

Polynucleotides described herein are used for a variety of purposes, such as probes for detection of and/or measurement of, transcription levels of a polynucleotide that is differentially expressed in a cancer cell. A probe that hybridizes or amplifies specifically a polynucleotide disclosed herein should provide a detection signal at least 2-, 5-, 10-, or 20-fold higher than the background hybridization provided with other unrelated sequences. It should be noted that “probe” as used in this context of detection of nucleic acid is meant to refer to a polynucleotide sequence used to detect a differentially expressed gene product in a test sample. As will be readily appreciated by the ordinarily skilled artisan, the probe can be detectably labeled and contacted with, for example, an array comprising immobilized polynucleotides obtained from a test sample (e.g., RNA). Alternatively, the probe can be immobilized on an array and the test sample detectably labeled. These and other variations of the methods of the invention are well within the skill in the art and are within the scope of the invention.

Labeled nucleic acid probes may be used to detect expression of a gene corresponding to the provided polynucleotide, e.g. in a macroarray format, Northern blot, etc. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes may be labeled with a radioactive isotope. Other types of detectable labels can be used such as chromophores, fluorophores, and enzymes.

PCR is another means for detecting small amounts of target nucleic acids, methods for which may be found in Sambrook, et al. Molecular Cloning: A Laboratory Manual, CSH Press 1989, pp. 14.2-14.33. A detectable label may be included in the amplification reaction. The label may be conjugated to one or both of the primers. Alternatively, the pool of nucleotides used in the amplification is labeled, so as to incorporate the label into the amplification product.

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

### Diagnosis, Prognosis, Assessment of Therapy (Therametrics), and Management of Cancer

The polynucleotides described herein are of particular interest as genetic or biochemical markers (e.g., in blood or tissues) that will detect the changes along the carcinogenesis pathway and/or to monitor the efficacy of various therapies and preventive interventions.

For example, the level of expression of certain polynucleotides can be indicative of a poorer prognosis, and therefore warrant more aggressive chemo- or radio-therapy for a patient or vice versa. The correlation of novel surrogate tumor specific features with response to treatment and outcome in patients can define prognostic indicators that allow the design of tailored therapy based on the molecular profile of the tumor.

Determining expression of certain polynucleotides and comparison of a patient's profile with known expression in normal tissue and variants of the disease allows a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

The polynucleotides that correspond to differentially expressed genes, as well as their encoded gene products, can be useful to monitor patients having or susceptible to cancer to detect potentially malignant events at a molecular level before they are detectable at a gross morphological level. In addition, the polynucleotides described herein, as well as the genes corresponding to such polynucleotides, can be useful as therametrics, e.g., to assess the effectiveness of therapy by using the polynucleotides to assess, for example, tumor burden in the patient before, during, and after therapy.

Furthermore, a polynucleotide identified as corresponding to a gene that is differentially expressed in, and thus is important for, one type of cancer can also have implications for development or risk of development of other types of cancer, e.g., where a polynucleotide represents a gene differentially expressed across various cancer types. Thus, for example, expression of a polynucleotide corresponding to a gene that has clinical implications for SCCC might also have clinical implications for metastatic breast cancer, colon cancer, or ovarian cancer, etc.

Staging. Staging is a process used by physicians to describe how advanced the cancerous state is in a patient. Staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

The polynucleotides and corresponding genes and gene products described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of cancers. Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well-differentiated or low-grade tumors.

The polynucleotides, and their corresponding genes and gene products, can be especially valuable in determining the grade of the tumor, as they not only can aid in determining the differentiation status of the cells of a tumor, they can also identify factors other than differentiation that are valuable in determining the aggressiveness of a tumor, such as metastatic potential. Low grade means that the cancer cells look very like the normal cells. They are usually slowly growing and are less likely to spread. In high grade tumors the cells look very abnormal. They are likely to grow more quickly and are more likely to spread.

Assessment of proliferation of cells in tumor. The differential expression level of the polynucleotides described herein can facilitate assessment of the rate of proliferation of tumor cells, and thus provide an indicator of the aggressiveness of the rate of tumor growth. For example, assessment of the relative expression levels of genes involved in cell cycle can provide an indication of cellular proliferation, and thus serve as a marker of proliferation.

### Detection of Cancer.

The polynucleotides corresponding to genes that exhibit the appropriate expression pattern can be used to detect cancer in a subject. The expression of appropriate polynucleotides can be used in the diagnosis, prognosis and management of cancer. Detection of cancer can be determined using expression levels of any of these sequences alone or in combination with the levels of expression of other known cancer genes. Determination of the aggressive nature and/or the metastatic potential of a cancer can be determined by comparing levels of one or more gene products of the genes corresponding to the polynucleotides described herein, and comparing total levels of another sequence known to vary in cancerous tissue. Expression of specific marker polynucleotides can be used to discriminate between normal and cancerous tissue, to discriminate between cancers with different cells of origin, to discriminate between cancers with different potential metastatic rates, etc. For a review of other markers of cancer, see, e.g., Hanahan et al. (2000) Cell 100:57-70.

### Treatment of Cancer

The invention further provides methods for reducing growth of cancer cells. The methods provide for decreasing the expression of a gene that is differentially expressed in a cancer cell or decreasing the level of and/or decreasing an activity of a cancer-associated polypeptide. In general, the methods comprise contacting a cancer cell with a substance that modulates expression of a gene that is differentially expressed in cancer; or a level of and/or an activity of a cancer-associated polypeptide.

“Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with cervical cancer, etc.

The present invention provides methods for treating cancer, generally comprising administering to an individual in need thereof a substance that reduces cancer cell growth, in an amount sufficient to reduce cancer cell growth and treat the cancer. Whether a substance, or a specific amount of the substance, is effective in treating cancer can be assessed using any of a variety of known diagnostic assays for cancer, including, but not limited to, proctoscopy, rectal examination, biopsy, contrast radiographic studies, CAT scan, and detection of a tumor marker associated with cancer in the blood of the individual. The substance can be administered systemically or locally. Thus, in some embodiments, the substance is administered locally, and cancer growth is decreased at the site of administration. Local administration may be useful in treating, e.g., a solid tumor.

A substance that reduces cancer cell growth can be targeted to a cancer cell. Thus, in some embodiments, the invention provides a method of delivering a drug to a cancer cell, comprising administering a drug-antibody complex to a subject, wherein the antibody is specific for a cancer-associated polypeptide, and the drug is one that reduces cancer cell growth, a variety of which are known in the art. Targeting can be accomplished by coupling (e.g., linking, directly or via a linker molecule, either covalently or non-covalently, so as to form a drug-antibody complex) a drug to an antibody specific for a cancer-associated polypeptide. Methods of coupling a drug to an antibody are well known in the art and need not be elaborated upon herein.

### Tumor Classification and Patient Stratification

The invention further provides for methods of classifying tumors, and thus grouping or “stratifying” patients, according to the expression profile of selected differentially expressed genes in a tumor. Differentially expressed genes can be analyzed for correlation with other differentially expressed genes in a single tumor type or across tumor types. Genes that demonstrate consistent correlation in expression profile in a given cancer cell type (e.g., in a cancer cell or type of cancer) can be grouped together, e.g., when one gene is overexpressed in a tumor, a second gene is also usually overexpressed. Tumors can then be classified according to the expression profile of one or more genes selected from one or more groups.

The tumor of each patient in a pool of potential patients can be classified as described above. Patients having similarly classified tumors can then be selected for participation in an investigative or clinical trial of a cancer therapeutic where a homogeneous population is desired. The tumor classification of a patient can also be used in assessing the efficacy of a cancer therapeutic in a heterogeneous patient population. In addition, therapy for a patient having a tumor of a given expression profile can then be selected accordingly.

The invention also encompasses the selection of a therapeutic regimen based upon the expression profile of differentially expressed genes in the patient's tumor. For example, a tumor can be analyzed for its expression profile of the genes described herein, e.g., the tumor is analyzed to determine which genes are expressed at elevated levels or at decreased levels relative to normal cells of the same tissue type. The expression patterns of the tumor are then compared to the expression patterns of tumors that respond to a selected therapy. Where the expression profiles of the test tumor cell and the expression profile of a tumor cell of known drug responsivity at least substantially match (e.g., selected sets of genes at elevated levels in the tumor of known drug responsivity and are also at elevated levels in the test tumor cell), then the therapeutic agent selected for therapy is the drug to which tumors with that expression pattern respond.

### Pattern Matching in Diagnosis Using RNA Sequencing and Microarrays

In another embodiment, the diagnostic and/or prognostic methods of the invention involve detection of expression of a selected set of small RNA sequences in a test sample to produce a test expression pattern. The test expression pattern is compared to a reference expression pattern, which is generated by detection of expression of the selected set of genes in a reference sample (e.g., a positive or negative control sample). The selected set of genes includes at least one of the small RNA sequences described herein.

The present invention also encompasses methods for identification of agents having the ability to modulate activity of a differentially expressed gene product, as well as methods for identifying a differentially expressed small RNA sequence as a therapeutic target for treatment of cancer.

Identification of compounds that modulate activity of a differentially expressed small RNA sequence can be accomplished using any of a variety of drug screening techniques. Such agents are candidates for development of cancer therapies. Of particular interest are screening assays for agents that have tolerable toxicity for normal, non-cancerous human cells. The screening assays of the invention are generally based upon the ability of the agent to modulate an activity of a differentially expressed gene product and/or to inhibit or suppress phenomenon associated with cancer (e.g., cell proliferation, colony formation, cell cycle arrest, metastasis, and the like).

Screening assays can be based upon any of a variety of techniques readily available and known to one of ordinary skill in the art. In general, the screening assays involve contacting a cancerous cell with a candidate agent, and assessing the effect upon biological activity of a differentially expressed small RNA sequence. The effect upon a biological activity can be detected by, for example, detection of expression. Alternatively or in addition, the effect of the candidate agent can be assessed by examining the effect of the candidate agent in a functional assay. In general, where the differentially expressed gene is increased in expression in a cancerous cell, agents of interest are those that decrease activity of the differentially expressed gene product.

Exemplary assays useful in screening candidate agents include, but are not limited to, hybridization-based assays (e.g., use of nucleic acid probes or primers to assess expression levels), and the like. Additional exemplary assays include, but are not necessarily limited to, cell proliferation assays, antisense knockout assays, assays to detect inhibition of cell cycle, assays of induction of cell death/apoptosis, and the like. Generally such assays are conducted in vitro, but many assays can be adapted for in vivo analyses, e.g., in an animal model of the cancer.

The term “agent” as used herein describes any molecule, e.g. protein or pharmaceutical, with the capability of modulating a biological activity of a gene product of a differentially expressed gene. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts (including extracts from human tissue to identify endogenous factors affecting differentially expressed gene products) are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature.

The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation is determined by routine experimentation and is within the judgment of the clinician. For purposes of the present invention, an effective dose will generally be from about 0.01 μg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, lipid aggregates and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles.

Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The dose and the means of administration of the inventive pharmaceutical compositions are determined based on the specific qualities of the therapeutic composition, the condition, age, and weight of the patient, the progression of the disease, and other relevant factors. For example, administration of polynucleotide therapeutic composition agents includes local or systemic administration, including injection, oral administration, particle gun or catheterized administration, and topical administration.

Also provided by the subject invention are kits for practicing diagnostic and therapeutic methods. The subject kits include at least one or more of: a subject nucleic acid probe that specifically hybridizes to at least one small RNA sequence described herein, and may comprise two probes for each small RNA, and may comprise probes specific for two, three, four, five or more different small RNA sequences. Other optional components of the kit include: control primers and plasmids; buffers, cells, carriers, adjuvants etc. The various components of the kit may be present in separate containers or certain compatible components may be precombined into a single container, as desired. In certain embodiments, controls, such as samples from a cancerous or non-cancerous cell are provided by the invention.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Patterns of Known and Novel Small RNAs in Human Cervical Cancer

Recent studies suggest that knowledge of differential expression of miRNAs in cancer may have substantial diagnostic and prognostic value. Here, a direct sequencing method is used to characterize the profiles of miRNAs and other small RNA segments for six human cervical carcinoma cell lines and five normal cervical samples. Of 166 miRNAs expressed in normal cervix and cancer cell lines, we observed significant expression variation of six miRNAs between the two groups. To further demonstrate the biological relevance of our findings, we examined the expression level of two significantly varying miRNAs in a panel of 29 matched pairs of human cervical cancer and normal cervical samples. Reduced expression of miR-143 and increased expression of miR-21 were reproducibly displayed in cancer samples, demonstrating the value of these miRNAs as tumor markers. In addition to the known miRNAs, we found a number of novel miRNAs and an additional set of small RNAs that do not meet miRNA criteria.

Direct sequencing was used to document miRNAs profiles and to identify novel miRNAs in six human cervical cancer cell lines and five normal cervical samples. In addition to a large family of known miRNAs, this approach resulted in the identification of a number of novel small RNA effectors. While there are clear similarities between the six cancer cell lines and normal cervix for known miRNAs, there are also clear differences. Our findings demonstrate a number of specific and consistent alterations in the small RNA profile in cervical cancer.

### Materials and Methods

Cell culture. Six human cervical carcinoma cell lines (SW756, C4I, C33A, CaSki, SiHa and ME-180) were cultured in DMEM medium supplemented with 10% fetal bovine serum and 1% penicillin/streptomycin (Invitrogen, Carlsbad, Calif.) at 37° C. and 5% CO₂ in a humidified incubator.

Clinical samples. Five histopathologically verified normal cervical samples were obtained from patients who underwent total hysterectomy because of benign gynecologic pathology. The tissues were snap frozen in liquid nitrogen immediately after surgical removal and stored at −70° C. until required. In addition, 29 pairs of snap-frozen cervical tumor and matched normal tissue were obtained from the Gynecologic Oncology Group Tissue Bank (Columbus, Ohio). Of these 29 cases with paired specimens, 19 patients had a diagnosis of squamous cell carcinoma, 7 had adenocarcinoma, 2 had adenosquamous cell carcinoma and 1 had small cell carcinoma. The matched normal cervical tissues were obtained from the same patients. The use of all specimens in the study was approved by the Institutional Review Board of Stanford University.

Small RNA isolation and cloning. Small RNA was extracted using mirVana miRNA isolation kit (Ambion, Austin, Tex.). The cloning was performed as described by Lau et al. Science 2001; 294:858-62, with slight modifications. Purified small RNAs were incubated with 10 μM pre-adenylylated 3′-adaptor oligonucleotide (“Modban”, IDT Inc., Coralville, Iowa), 1× adenylate ligation buffer (ATP-free), 10% DMSO and 1 U T4 RNA ligase (New England Biolabs, Ipswich, Mass.) at 37° C. for 1 hour. The ligated product was purified on a 12% denaturing polyacrylamide gel, followed by a second ligase reaction with a 5′-adaptor oligonucleotide, 5′-ACGGAATTCCTCACTaaa-3′ (SEQ ID NO:1) (uppercase, DNA; lowercase, RNA; ChemGenes Corporation, Wilmington, Mass.) and gel-purification. The gel-purified doubly ligated RNA was reversed transcribed using 150 U Superscript II (Invitrogen) and RT primer, 5′-ATTGATGGTGCCTACAG-3′ (SEQ ID NO:2). The cDNA was amplified by PCR, using the RT primer and a forward primer 5′-CAGCCAACGGAATTCCTCACTAAA-3 (SEQ ID NO:3). A second PCR was performed using the RT primer and a second forward primer, 5′-GAGCCAACAGGCACCGAATTCCTCACTAAA-3′ (SEQ ID NO:4). The PCR product was phenol-extracted, ethanol-precipitated, and then digested with Ban I (NEB). After further phenol extraction and ethanol precipitation, the digested products were concatemerized with T4 DNA ligase (NEB). Concatamers ranging from 600-1000 by were isolated from a low-melting-point agarose gel, processed with Taq polymerase, and cloned into the pCR4-TOPO vector using the TOPO TA cloning kit (Invitrogen). Colony PCR was performed using the M13 forward and reverse primers, and the PCR products were purified using shrimp alkaline phosphatase and exonuclease I (USB Corporation, Cleveland, Ohio).

Sequence analysis. Small RNAs obtained by cloning were compared with functionally annotated sequences using BLAST, BLAT from the University of California at Santa Cruz, miRBase, as well as standard text-matching routines. For each small RNA, the best alignments to a functionally annotated sequence (not more than one error) were used to assign a functional category to the small RNA. The fold-back precursor structure of candidate miRNAs was predicted by mfold.

Statistical analysis. To determine if any significant variation in miRNA expression profiles was found between the cancer cell lines and normal cervix, we applied χ²-statistics to compare the two groups. P<0.0001 was considered as significant.

Northern analysis. One microgram of small RNA was fractionated on denaturing 15% polyacrylamide gels, followed by staining with ethidium bromide to determine RNA integrity. Overall RNA levels also served as internal loading controls. Gels were then destained, transferred to Hybond-N+ membranes (Amersham Biosciences) using semi-dry transfer (Hoefer Scientific Instruments, San Francisco, Calif.) and fixed by ultraviolet cross-linking at 1200 μjoules and baking at 80° C. for 1 hour. Membranes were hybridized overnight at 55° C. in PerfectHyb™ Plus hybridization buffer (Sigma), together with a LNA-modified oligonucleotide probe complementary to the mature miR-143 (5′-TGAGCTACAGTGCTTCATCTCA (SEQ ID NO:5); bold, LNA; IDT) or miR-21 (5′-TCAACATCAGTCTGATAAGCTA (SEQ ID NO:6); bold, LNA, IDT) that was labeled with terminal transferase (NEB) and biotin-16-dUTP (Roche Diagnostics). Subsequently, the blots were washed at 55° C. for 15 min each in 2×SSC/0.1% SDS and 0.2×SSC/0.1% SDS, followed by an incubation in blocking solution (1×PBS, pH 7.4/0.05% Tween 20/0.1% SDS/0.5% blocking reagent, Roche) for 1 hour and then in streptavidin-alkaline phosphatase conjugate (USB Corp) for 1 hour. After incubation, the blots were washed three times in each buffer A (1×PBS, pH 7.4/0.05% Tween 20/0.1% SDS) and buffer B (0.1 M Tris-HCl, pH 9.5, 0.1 M NaCl). The blots were then incubated with chemiluminescent substrate CDP-Star™ (GE Healthcare, Piscataway, N.J.) and exposed to Kodak BioMax XAR film.

### Results

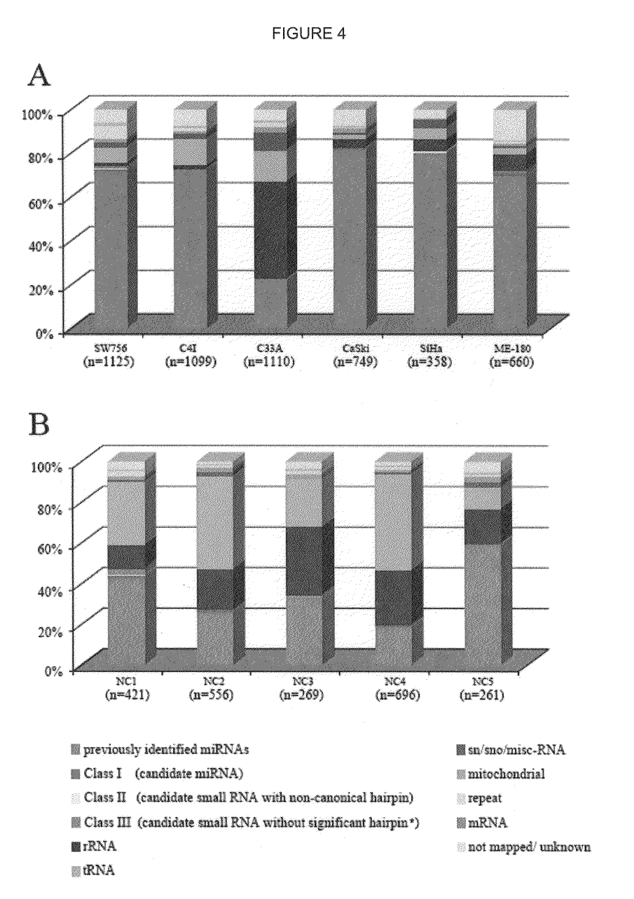

Small RNA composition of cDNA libraries. To search for novel candidate miRNAs or other small RNAs and to characaterize miRNAs in human cervical cancer and normal cervix, we cloned and sequenced small RNA libraries prepared from RNA in the size range of 18-25 nt isolated from six human cervical cancer cell lines and five normal cervices. A total of 7303 small RNA clones (5100 from cervical cancer cell lines and 2203 from normal cervix) were sequenced (FIG. 4, Tables 4-5).

TABLE 4

| | SW756 | | C4I | | C33A | | CaSki | | SiHa | | ME-180 | |
|---|---|---|---|---|---|---|---|---|---|---|---|---|
| | no. of | | no. of | | no. of | | no. of | | no. of | | no. of | |
| Type | clones | % | clones | % | clones | % | clones | % | clones | % | clones | % |
| previously identified miRNAs | 815 | 72.4 | 799 | 72.7 | 248 | 22.3 | 617 | 82.4 | 288 | 80.4 | 464 | 70.3 |
| Novel small RNAs | | | | | | | | | | | | |
| Class I (candidate miRNA) | 8 | 0.7 | 3 | 0.3 | 1 | 0.1 | 3 | 0.4 | 0 | 0.0 | 7 | 1.1 |
| Class II (candidate small RNA | 3 | 0.3 | 0 | 0.0 | 1 | 0.1 | 0 | 0.0 | 3 | 0.8 | 0 | 0.0 |
| with non-canonical hairpin) | | | | | | | | | | | | |
| Class III (candidate small RNA | 7 | 0.6 | 1 | 0.1 | 4 | 0.4 | 1 | 0.1 | 0 | 0.0 | 5 | 0.8 |
| without significant hairpin) | | | | | | | | | | | | |
| rRNA | 22 | 2.0 | 18 | 1.6 | 495 | 44.6 | 29 | 3.9 | 19 | 5.3 | 52 | 7.9 |
| tRNA | 78 | 6.9 | 131 | 11.9 | 156 | 14.1 | 17 | 2.3 | 19 | 5.3 | 20 | 3.0 |
| [symbol] -RNA | 28 | 2.5 | 27 | 2.5 | 94 | 8.5 | 6 | 0.8 | 14 | 3.9 | 5 | 0.8 |
| mitochondrial | 14 | 1.2 | 13 | 1.2 | 23 | 2.1 | 13 | 1.7 | 2 | 0.6 | 7 | 1.1 |
| repeat | 71 | 6.3 | 17 | 1.5 | 22 | 2.0 | 6 | 0.8 | 2 | 0.6 | 5 | 0.8 |
| mRNA | 13 | 1.2 | 10 | 0.9 | 13 | 1.2 | 3 | 0.4 | 0 | 0.0 | 4 | 0.6 |
| not mapped unknown | 66 | 5.9 | 80 | 7.3 | 53 | 4.8 | 54 | 7.2 | 11 | 3.1 | 91 | 13.8 |
| No. sec. | 1125 | 100 | 1099 | 100 | 1110 | 100 | 749 | 100 | 358 | 100 | 660 | 100 |

[symbol]

indicates data missing or illegible when filed

TABLE 5

| | NC1 | | NC2 | | NC3 | | NC4 | | NC5 | |
|---|---|---|---|---|---|---|---|---|---|---|
| | no. of | | no. of | | no. of | | no. of | | no. of | |
| Type | clones | % | clones | % | clones | % | clones | % | clones | % |
| previously identified miRNAs | 183 | 43.5 | 147 | 26.4 | 89 | 33.3 | 132 | 19.3 | 155 | 59.4 |
| Novel small RNAs | | | | | | | | | | |
| Class I (candidate miRNA) | 1 | 0.2 | 3 | 0.5 | 0 | 0.0 | 1 | 0.1 | 0 | 0.0 |
| Class II (candidate small RNA | 3 | 0.7 | 0 | 0.0 | 2 | 0.7 | 0 | 0.0 | 0 | 0.0 |
| with non-canonical hairpin) | | | | | | | | | | |
| Class III (candidate small RNA | 11 | 2.6 | 0 | 0.0 | 3 | 1.1 | 5 | 0.7 | 1 | 0.4 |
| without significant hairpin) | | | | | | | | | | |
| rRNA | 51 | 12.1 | 113 | 20.3 | 90 | 33.7 | 186 | 27.2 | 45 | 17.2 |
| tRNA | 131 | 31.1 | 252 | 45.3 | 63 | 23.6 | 323 | 47.3 | 27 | 10.3 |
| [symbol] -RNA | 5 | 1.2 | 13 | 2.3 | 1 | 0.4 | 9 | 1.3 | 7 | 2.7 |
| mitochondrial | 5 | 1.2 | 11 | 2.0 | 5 | 1.9 | 5 | 0.7 | 7 | 2.7 |
| repeat | 13 | 3.1 | 7 | 1.3 | 4 | 1.5 | 10 | 1.5 | 3 | 1.1 |
| mRNA | 2 | 0.5 | 3 | 0.5 | 3 | 1.1 | 5 | 0.7 | 3 | 1.1 |
| not mapped unknown | 16 | 3.8 | 7 | 1.3 | 9 | 3.4 | 20 | 2.9 | 13 | 5.0 |
| No. sec. | 421 | 100 | 556 | 100 | 269 | 101 | 696 | 102 | 261 | 100 |

[symbol]

indicates data missing or illegible when filed

For cervical cancer cell lines, a total of 3231 (63%) could be annotated as known miRNAs (Table 6). This corresponds to 93 previously verified miRNAs, 17 miRNAs that had been computationally predicted but never verified experimentally and 25 sequences paired with known mature miRNAs that have not been annotated in miRBase. The majority of the remaining small RNAs correspond to fragments of rRNA (12%), tRNA (8%), scRNA/snRNA (3%), repeat sequences (2%), mitochondrial (1%), and mRNA (1%) (FIG. 4A, Table 4). One cell line in particular (C33A) had a higher fraction of ribosomal RNAs. This may result from a high fraction of dying cells in this line (site-specific degradation of rRNAs is a feature of dying cells). A total of 46 clones (1%) could be aligned to the human genome sequences but did not correspond to any annotated RNA species. Twenty-one of these small RNA clones were designated as novel candidate miRNAs, based on a predicted hairpin precursor with the following properties (Table 1): (1) complete containment of the cDNA sequence within one arm of a hairpin, (2) at least 16 nucleotides of the cDNA sequence involved in base-pairing, and (3) identification as the lowest free energy structure by mfold. Another seven clones (0.1%) fulfilled criteria 1 and 3 but had somewhat fewer duplex base pairs in the hairpin region (Table 2). The remaining aligned but non-annotated clones showed poor secondary fold-back hairpin structure predicted by mfold or could not fulfill the criteria listed above (Table 3). The final class of sequences from the libraries was a fraction that could not be aligned at any point in the reference human genome sequence (7%; 355 of 5101). These could reflect patient-specific, tumor-specific, or cell line-specific sequence polymorphisms, non-annotated regions in the human genome, PCR/cloning/sequencing errors, or non-human biological material present in the culture. Interestingly, none of the small RNA sequences that were identified appeared to correspond to human papillomavirus RNA, despite the fact that five of the cell lines are HPV-positive.

TABLE 2

| Novel small RNAs with non-canonical hairpin identified from human cervical cancer cell lines and normal cervical samples | | | | | | | | | | | | | | | |
|---|---|---|---|---|---|---|---|---|---|---|---|---|---|---|---|
| | | sample | | | | | Conservation of fold-back | | | | | | | | |
| candidate | | (no. of | size | | | | structure^{d} | | | | | | | | |
| sRNA^{a} | sequence (5′ -> 3′)^{b} | clones) | (nt) | stem-loop structures of putative miRNA precursors^{c} | dG | location | ptr | mmu | rno | cfa | dre | gga | fru | dme | cel |
| sRNA-cer1 | CGAGGAGGCUCCCAGAGUGUGU | SW756 | 22 | -- - CG A CU A U A G | −35.2 | 2q33.1 | | | | yes | | | | | |
| | (SEQ ID NO: 42) | (2) | | UCCUG GUGA GC AGG GG CCC GAGUG GU UGGU U | | | | | | | | | | | |
| | | | | AGGAC CACU CG UCC CC GGG CUCAC CA ACUA G | | | | | | | | | | | |
| | | | | UA A -- C UC A U G G | | | | | | | | | | | |
| | | | | (SEQ ID NO: 43) | | | | | | | | | | | |
| sRNA-cer2 | AGCGAUGUGGGAAGGCUUGUG | SW756 | 21 | CC ------ ------ CC U UU | −44.8 | 8q22.3 | yes | | | | | | | | |
| | (SEQ ID NO: 44) | (1) | | CUG CUGCUCU GGCCUUCCU UUCCCACC GC GGG U | | | | | | | | | | | |
| | | | | GAC GACGAGG UCGGAAGGG GAGGGUGG CG UCU G | | | | | | | | | | | |
| | | | | UC AGGUGU UGUAGC A- - AC | | | | | | | | | | | |
| | | | | (SEQ ID NO: 45) | | | | | | | | | | | |
| sRNA-cer3 | ACUCUUAGCGGUGGAUCACUC | C33A | 21 | C - CCUC-- G CCC C G | −32.4 | 19p12 | yes | | | | | | | | |
| | (SEQ ID NO: 46) | (1) | | GUG CAGGAG GG CCG UGUUGGGAGU UCAC AAA A | | | | | | | | | | | |
| | | | | UAC GUCCUC CC GGU GCGAUUCUCA AGUG UUU A | | | | | | | | | | | |
| | | | | - A UCACUA G A-- - C | | | | | | | | | | | |
| | | | | (SEQ ID NO: 47) | | | | | | | | | | | |
| sRNA-cer4 | CAGAUGAGGAAGCCAUGGCUAGUU | SiHa | 24 | G --- - AAAA- AAAGAA AACC | −22 | 14q32.32 | yes | | | | | | | | |
| | (SEQ ID NO: 48) | (3) | | UGAGA GAGAGA GGCCA GG UUAUUU GCC \ | | | | | | | | | | | |
| | | | | AUUUU UUUUCU UCGGU CC AGUAGA CGG A | | | | | | | | | | | |
| | | | | G UGA A GAAGG CAA--- ACUU | | | | | | | | | | | |
| | | | | (SEQ ID NO: 49) | | | | | | | | | | | |
| sRNA-cer5 | ACCCGUCCCGUUCGUCCCCGGA | NC1 | 22 | A C C UU C AC UGC | −44.7 | 14q32.31 | | yes | yes | | | | | | |
| | (SEQ ID NO: 50) | (1) | | GGGCGC CC GUC CG CGU CCCGG GU U | | | | | | | | | | | |
| | | | | CCCGCG GG CAG GC GCA GGGCC CA C | | | | | | | | | | | |
| | | | | A U A U- A C- UCU | | | | | | | | | | | |
| | | | | (SEQ ID NO: 51) | | | | | | | | | | | |
| sRNA-cer6 | CUCAGGGUGACGGUGAGCAG | NC1 | 20 | UAACUCA C A G - C ACAAG | −33.3 | 3p12.3 | | | | | | | | | |
| | (SEQ ID NO: 52) | (1) | | GUGGG GGGUGA GGUG GCA UGC CACU uCAUuUU \ | | | | | | | | | | | |
| | | | | CACCU UUUACU UCAC CGU ACG GUGG AGUGGGA U | | | | | | | | | | | |
| | | | | ------- C - G A U CUCAA | | | | | | | | | | | |
| | | | | (SEQ ID NO: 53) | | | | | | | | | | | |
| sRNA-cer7 | CCCAGAUGGUUCCAGUUUGU | NC1 | 20 | - A UAUAG UUUU- CA GAAA | −20.3 | Xp22.2 | yes | | | | | | | | |
| | (SEQ ID NO: 54) | (1) | | CAAA UGGAGC CAUCU GAGUC GU A | | | | | | | | | | | |
| | | | | GUUU ACCUUG GUAGA CUCAG CA U | | | | | | | | | | | |
| | | | | U G ----- CCCUC A- AAUA | | | | | | | | | | | |
| | | | | (SEQ ID NO: 55) | | | | | | | | | | | |
| sRNA-cer8 | CAUAAAUGAAUGGCUGGGGCAA | NC3 | 22 | G G AG- CCA--- UUAUG | −26.5 | 8p21.1 | | | | | | | | | |
| | (SEQ ID NO: 56) | (1) | | GCCU GCCU GGC UCA GGA A | | | | | | | | | | | |
| | | | | CGGA CGGG UCG AGU CCU U | | | | | | | | | | | |
| | | | | A G GUA AAAUAC UUCUC | | | | | | | | | | | |
| | | | | (SEQ ID NO: 57) | | | | | | | | | | | |
| sRNA-cer9 | CCACUAGUAUGAUCAGAGCCUC | NC3 | 22 | G ACCC-- ---- CU- U G A CU UGG | −31.1 | 16q24.2 | | | | | | | | | |
| | (SEQ ID NO: 58) | (1) | | GGA GUC CAC CCCCA AG AU AUCAG GC CCUG \ | | | | | | | | | | | |
| | | | | CCU CGG GUG GGGGU UC UG UAGUC CG GGAC G | | | | | | | | | | | |
| | | | | G GCACAC UAGA ACU - - G UC CCA | | | | | | | | | | | |
| | | | | (SEQ ID NO: 59) | | | | | | | | | | | |

^{a}sRNA-cer refers to small RNAs in cervical cells
^{b}Sequences listed represent the observed full length sequence of each sRNA cloned.
^{c}RNA secondary structure prediction was performed using mfold version 3.2. The sRNA sequence is underlined. The actual size of the stem-loop has not been experimentally determined.
^{d}ptr, Pan troglodytes (Chimpanzee); mmu, Mus musculus (mouse); rno, Rattus norregicus (rat); cfa, Canis familiaris (dog); dre, Dunio rerio (zebrafish); gga, Gallus gallus (chicken); fru, Fugu rubripes (pufferfish); dme, Drosophila melanogaster (fruitfly); cel, Caenorhabditis elegans (nematode)

TABLE 1

| | | | | | | | Conservation of fold-back | | | | | | | | |
|---|---|---|---|---|---|---|---|---|---|---|---|---|---|---|---|
| candidate | | no. of | size | | | | structure^{c} | | | | | | | | |
| miRNA | sequence (5′ -> 3′)^{a} | clones | (nt) | stem-loop structures of putative miRNA precursors^{b} | dG | location | ptr | mmt | rno | cfa | dre | gga | fru | dme | cel |
| miR-938 | UGCCCUUAAAGGUGAACCCAGU | 1 | 22 | U GCC A CA- G CC | −36 | 10p11.2 | yes | | | | | | | | |
| | | | | GAAGGUGUACCA GU CUUAAAGGUG ACC GU CA U | | | | | | | | | | | |
| | | | | CUUCCGUAUGGU CA GAAUUUCCAC UGG CA GU U | | | | | | | | | | | |
| | | | | U A-- A UGC A AC | | | | | | | | | | | |
| miR-939 | UGGGGAGCUGAGGCUCUGGGGGUC | 1 | 24 | GCA C U U UG GU C | −63 | 8q24.3 | yes | | | | | | | | |
| | (SEQ ID NO: 25) | | | UGUGG GGGC CUGGGGAGC GAGGC C GGG GGC G | | | | | | | | | | | |
| | | | | GCGCC UCUG GACCCCUCG CUCCG G CCC UCG G | | | | | | | | | | | |
| | | | | AG- U U - GU AG G | | | | | | | | | | | |
| | | | | (SEQ ID NO: 26) | | | | | | | | | | | |
| miR-940 | AAGGCAGGGCCCCCGCUCCCC | 2 | 20-21 | A G CCCCA - GG AG- -- GUGU | −55 | 16p13.3 | yes | | | yes | | | | | |
| | (SEQ ID NO: 27) | | | GUG GGU UGGGCCCGG GGAGCGGGG CCU GC CC CC \ | | | | | | | | | | | |
| | | | | CAC CCA GUCCGGGCC CCUCGCCCC GGG CG GG GG G | | | | | | | | | | | |
| | | | | C - ----- C A- GAA AA AGUU | | | | | | | | | | | |
| | | | | (SEQ ID NO: 28) | | | | | | | | | | | |
| miR-941 | CACCCGGCUGUGUGCACAUGUGC | 1 | 23 | - G U - C G C ACA - ACG | −53 | 20q13.3 | yes | | | | | | | | |
| | (SEQ ID NO: 29) | | | CCCGG CU UG GG ACAUGUGC CA GGCC GGG GCG CC G | | | | | | | | | | | |
| | | | | GGGCC GG AC CC UGUACACG GU UCGG CCC CGC GG A | | | | | | | | | | | |
| | | | | C G - G U G - A-- A AGA | | | | | | | | | | | |
| | | | | (SEQ ID NO: 30) | | | | | | | | | | | |
| miR-942 | UCUUCUCUGUUUUGGCCAUGUG | 1 | 22 | U UACUCA | −57 | 1p13.1 | yes | | | | | | | | |
| | (SEQ ID NO: 31) | | | AUUAGGAGAGUA CUUCUCUGUUUUGGCCAUGUGUG C | | | | | | | | | | | |
| | | | | UAAUCCUUUCAU GAAGAGACAAAGCCGGUACACAC A | | | | | | | | | | | |
| | | | | U UCCCCG | | | | | | | | | | | |
| | | | | (SEQ ID NO: 32) | | | | | | | | | | | |
| miR-943 | CUGACUGUUGCCGUCCUCCAG | 1 | 21 | C C- CUC - UUU -- CU UGC | −43 | 4p16.3 | yes | | | | | | | | |
| | (SEQ ID NO: 33) | | | GGGA GUU UGAG GGGG UGGGGGACG GC CGGUCA GC \ | | | | | | | | | | | |
| | | | | CCCU CGG ACUC CCCC ACCUCCUGC CG GUCAGU CG U | | | | | | | | | | | |
| | | | | A AA A-- G --- UU CC CGG | | | | | | | | | | | |
| | | | | (SEQ ID NO: 34) | | | | | | | | | | | |
| miR-944 | AAAUUAUUGUACAUCGGAUGAG | 1 | 22 | U A U AAAUU | −46 | 3q28 | yes | | | | | | | | |
| | (SEQ ID NO: 35) | | | GUUCCAGACACA CUCAUCUGAU UACAAUA UUUCUU G | | | | | | | | | | | |
| | | | | UAGGGUCUGUGU GAGUAGGCUA AUGUUAU AAAGAG U | | | | | | | | | | | |
| | | | | C C U AAAUA | | | | | | | | | | | |
| | | | | (SEQ ID NO: 36) | | | | | | | | | | | |
| miR-708 | AAGGAGCUUACAAUCUAGCUGG | 2 | 22 | AA A A C GGG AA ACU | −48 | 11q14.1 | yes | yes | yes | yes | | | | | |
| | (SEQ ID NO: 37) | | | GGU CUGCCCUC AGGAGCUUACA UCUAG UG GU AUG \ | | | | | | | | | | | |
| | | | | CCA GACGGGAG UCUUCGAGUGU AGAUC AC CA UAC U | | | | | | | | | | | |
| | | | | GG A C A A-- AG ACG | | | | | | | | | | | |
| | | | | (SEQ ID NO: 38) | | | | | | | | | | | |
| miR-874-5p | CGGCCCCACGCACCAGGGUAAG | 1 | 22 | CUG C A CA A A- AC | −45 | 5q31.2 | yes | yes | yes | yes | | | | | |
| miR-874-3p | CUGCCCUGGCCCGAGGGACCG | 1 | 21 | CGG CCC CG CCAGGGUA GAG G \ | | | | | | | | | | | |
| | (SEQ ID NO: 39-40) | | | GCC GGG GC GGUCCCGU CUU C U | | | | | | | | | | | |
| | | | | --- A A CC C CG UC | | | | | | | | | | | |
| | | | | (SEQ ID NO: 41) | | | | | | | | | | | |

^{a}Sequences listed represent the observed full length sequence of each miRNA cloned. The extra bases at the termini of some miRNAs are denoted in bold.
^{b}RNA secondary structure prediction was performed using mfold version 3.2. The miRNA sequence is underlined. The actual size of the stem-loop has not been experimentally determined.
^{c}ptr, Pan troglodytes (Chimpanzee); mmu, Mus musculus (mouse); rno, Rattus norvegicus (rat); cfa, Canis familiaris (dog); dre, Danio rerio (zebrafish); gga, Gallus gallus (chicken); fru, Fugu rubripes (pufferfish); dme, Drosophila melanogaster (fruitfly); cel, Caenorhabditis elegans (nematode)

TABLE 2

| Novel small RNAs with non-canonical hairpin identified from human cervical cancer cell lines and normal cervical samples | | | | | | | | | | | | | | | |
|---|---|---|---|---|---|---|---|---|---|---|---|---|---|---|---|
| | | sample | | | | | Conservation of fold-back | | | | | | | | |
| candidate | | (no. of | size | | | | structure^{d} | | | | | | | | |
| sRNA^{a} | sequence (5′ -> 3′)^{b} | clones) | (nt) | stem-loop structures of putative miRNA precursors^{c} | dG | location | ptr | mmu | rno | cfa | dre | gga | fru | dme | cel |
| sRNA-cer1 | CGAGGAGGCUCCCAGAGUGUGU | SW756 | 22 | -- - CG A CU A U A G | −35.2 | 2q33.1 | | | | yes | | | | | |
| | (SEQ ID NO: 42) | (2) | | UCCUG GUGA GC AGG GG CCC GAGUG GU UGGU U | | | | | | | | | | | |
| | | | | AGGAC CACU CG UCC CC GGG CUCAC CA ACUA G | | | | | | | | | | | |
| | | | | UA A -- C UC A U G G | | | | | | | | | | | |
| | | | | (SEQ ID NO: 43) | | | | | | | | | | | |
| sRNA-cer2 | AGCGAUGUGGGAAGGCUUGUG | SW756 | 21 | CC ------ ------ CC U UU | −44.8 | 8q22.3 | yes | | | | | | | | |
| | (SEQ ID NO: 44) | (1) | | CUG CUGCUCU GGCCUUCCU UUCCCACC GC GGG U | | | | | | | | | | | |
| | | | | GAC GACGAGG UCGGAAGGG GAGGGUGG CG UCU G | | | | | | | | | | | |
| | | | | UC AGGUGU UGUAGC A- - AC | | | | | | | | | | | |
| | | | | (SEQ ID NO: 45) | | | | | | | | | | | |
| sRNA-cer3 | ACUCUUAGCGGUGGAUCACUC | C33A | 21 | C - CCUC-- G CCC C G | −32.4 | 19p12 | yes | | | | | | | | |
| | (SEQ ID NO: 46) | (1) | | GUG CAGGAG GG CCG UGUUGGGAGU UCAC AAA A | | | | | | | | | | | |
| | | | | UAC GUCCUC CC GGU GCGAUUCUCA AGUG UUU A | | | | | | | | | | | |
| | | | | - A UCACUA G A-- - C | | | | | | | | | | | |
| | | | | (SEQ ID NO: 47) | | | | | | | | | | | |
| sRNA-cer4 | CAGAUGAGGAAGCCAUGGCUAGUU | SiHa | 24 | G --- - AAAA- AAAGAA AACC | −22 | 14q32.32 | yes | | | | | | | | |
| | (SEQ ID NO: 48) | (3) | | UGAGA GAGAGA GGCCA GG UUAUUU GCC \ | | | | | | | | | | | |
| | | | | AUUUU UUUUCU UCGGU CC AGUAGA CGG A | | | | | | | | | | | |
| | | | | G UGA A GAAGG CAA--- ACUU | | | | | | | | | | | |
| | | | | (SEQ ID NO: 49) | | | | | | | | | | | |
| sRNA-cer5 | ACCCGUCCCGUUCGUCCCCGGA | NC1 | 22 | A C C UU C AC UGC | −44.7 | 14q32.31 | yes | yes | | | | | | | |
| | (SEQ ID NO: 50) | (1) | | GGGCGC CC GUC CG CGU CCCGG GU U | | | | | | | | | | | |
| | | | | CCCGCG GG CAG GC GCA GGGCC CA C | | | | | | | | | | | |
| | | | | A U A U- A C- UCU | | | | | | | | | | | |
| | | | | (SEQ ID NO: 51) | | | | | | | | | | | |
| sRNA-cer6 | CUCAGGGUGACGGUGAGCAG | NC1 | 20 | UAACUCA C A G - C ACAAG | −33.3 | 3p12.3 | | | | | | | | | |
| | (SEQ ID NO: 52) | (1) | | GUGGG GGGUGA GGUG GCA UGC CACU UCAUUUU \ | | | | | | | | | | | |
| | | | | CACCU UUUACU UCAC CGU ACG GUGG AGUGGGA U | | | | | | | | | | | |
| | | | | ------- C - G A U CUCAA | | | | | | | | | | | |
| | | | | (SEQ ID NO: 53) | | | | | | | | | | | |
| sRNA-cer7 | CCCAGAUGGUUCCAGUUUGU | NC1 | 20 | - A UAUAG UUUU- CA GAAA | −20.3 | Xp22.2 | yes | | | | | | | | |
| | (SEQ ID NO: 54) | (1) | | CAAA UGGAGC CAUCU GAGUC GU A | | | | | | | | | | | |
| | | | | GUUU ACCUUG GUAGA CUCAG CA U | | | | | | | | | | | |
| | | | | U G ----- CCCUC A- AAUA | | | | | | | | | | | |
| | | | | (SEQ ID NO: 55) | | | | | | | | | | | |
| sRNA-cer8 | CAUAAAUGAAUGGCUGGGGCAA | NC3 | 22 | G G AG- CCA--- UUAUG | −26.5 | 8p21.1 | | | | | | | | | |
| | (SEQ ID NO: 56) | (1) | | GCCU GCCU GGC UCA GGA A | | | | | | | | | | | |
| | | | | CGGA CGGG UCG AGU CCU U | | | | | | | | | | | |
| | | | | A G GUA AAAUAC UUCUC | | | | | | | | | | | |
| | | | | (SEQ ID NO: 57) | | | | | | | | | | | |
| sRNA-cer9 | CCACUAGUAUGAUCAGAGCCUC | NC3 | 22 | G ACCC-- ---- CU- U G A CU UGG | −31.1 | 16q24.2 | | | | | | | | | |
| | (SEQ ID NO: 58) | (1) | | GGA GUC CAC CCCCA AG AU AUCAG GC CCUG \ | | | | | | | | | | | |
| | | | | CCU CGG GUG GGGGU UC UG UAGUC CG GGAC G | | | | | | | | | | | |
| | | | | G GCACAC UAGA ACU - - G UC CCA | | | | | | | | | | | |
| | | | | (SEQ ID NO: 59) | | | | | | | | | | | |

^{a}sRNA-cer refers to small RNAs in cervical cells
^{b}Sequences listed represent the observed full length sequence of each sRNA cloned.
^{c}RNA secondary structure prediction was performed using mfold version 3.2. The sRNA sequence is underlined. The actual size of the stem-loop has not been experimentally determined.
^{d}ptr, Pan Troglodytes (Chimpanzee); mmu, Mus musculus (mouse); rno, Rattus norvegicus (rat); cfa, Canis familiaris (dog); dre, Danio reria (zebrafish); gga, Gallus gallas (chicken); fru, Fugu rubripes (pufferfish); dme, Drosophila melanogaster (fruitfly); cel, Caenorhabditis elegans (nematode)

TABLE 3

| Candidate novel small RNAs without significant hairpin identified in human cervical cancer cell lines and normal cervix | | | | | | | | | | | | | | | | | | |
|---|---|---|---|---|---|---|---|---|---|---|---|---|---|---|---|---|---|---|
| | | | | | | Gene/intergenic^{c} | | | | | | | | | | | | |
| candidate | | size | chromo- | genomic location | | xon/intron/UTR | total | no. of clones | | | | | | | | | | |
| sRNA^{a} | sequence^{b} (SEQ ID NO:) | range | some | start | end | (S/A) | clones | SW756 | C4I | C33A | CaSki | SiHa | ME-18C | NC1 | NC2 | NC3 | NC4 | NC5 |
| sRNA-1 | AACUGAGGAUGGGAAAGC | 18 | 15q25.2 | 80126233 | 80126250 | intergenic | 1 | | | | | | 1 | | | | | |
| | (SEQ ID NO: 60) | | | | | | | | | | | | | | | | | |
| sRNA-2 | AUAAGCCCAGGAAAUGUGUAGAG | 23 | 10q25.1 | 108250081 | 1.08E+08 | intergenic | 2 | 2 | | | | | | | | | | |
| | (SEQ ID NO: 61) | | | | | | | | | | | | | | | | | |
| sRNA-3 | CCAAGCGAGGGAACAGUG | 18 | 17q12 | 30439716 | 30439733 | RFFL, intron (S) | 2 | 2 | | | | | | | | | | |
| | (SEQ ID NO: 62) | | | | | | | | | | | | | | | | | |
| sRNA-4 | UGGAGUAAGCCGGAUCGCG | 19 | 20q13.2 | 49612775 | 49612755 | intergenic | 1 | 1 | | | | | | | | | | |
| | (SEQ ID NO: 63) | | | | | | | | | | | | | | | | | |
| sRNA-5 | CCUCCUGGCGGGCAGCUGUG | 20 | 22q13.2 | 40621433 | 40621452 | SREBF2, intron (S) | 2 | | | 2 | | | | | | | | |
| | (SEQ ID NO: 64) | | | | | | | | | | | | | | | | | |
| sRNA-6 | CGCGGGUGCUUACUGACCCU | 20 | 5q31.2 | 135444088 | 1.35E+08 | intergenic | 1 | | | | 1 | | | | | | | |
| | (SEQ ID NO: 65) | | 10p11.22 | 31880040 | 31880059 | intergenic | | | | | | | | | | | | |
| sRNA-7 | CGGGUCGGAGUUAGCUCAAGCGG | 23 | 5q31.2 | 135444164 | 1.35E+08 | intergenic | 1 | | | | | | 1 | | | | | |
| | (SEQ ID NO: 66) | | 8q22.3 | 101674303 | 101674325 | intergenic | | | | | | | | | | | | |
| sRNA-8 | CUCUCUGCCCCUGGGAGAGAUCC | 23 | 9p13.3 | 33156003 | 33156025 | intergenic | 1 | | 1 | | | | | | | | | |
| | (SEQ ID NO: 67) | | | | | | | | | | | | | | | | | |
| sRNA-9 | GAGGUGGGAAAGGGAAGGGU | 20 | 20q11.23 | 34098787 | 34098804 | intergenic | 1 | 1 | | | | | | | | | | |
| | (SEQ ID NO: 68) | | 12p11.22 | 29436946 | 29436963 | intergenic | | | | | | | | | | | | |
| sRNA-10 | UAGAGGAGAUGGCGCAGGGGACAC | 21-24 | 6p21.33 | 30660089 | 30660112 | ABCF1, intron (S) | 2 | | | | | | 2 | | | | | |
| | (SEQ ID NO: 69) | | | | | | | | | | | | | | | | | |
| sRNA-11 | GUGGUGGUGGGGGAGGAGGAA | 21 | 1q24.3 | 168241442 | 168241462 | BAT2D1, exon (AS) | 1 | | | 1 | | | | | | | | |
| | (SEQ ID NO: 70) | | | | | | | | | | | | | | | | | |
| sRNA-12 | UCAAGGAGCUCACAAUCUAGU | 21 | 1p34.2 | 43489109 | 43489129 | MPL, 3′UTR (AS) | 1 | 1 | | | | | | | | | | |
| | (SEQ ID NO: 71) | | | | | | | | | | | | | | | | | |
| sRNA-13 | UCUGCCUCAACUCCGCCCCU | 20 | 19q13.33 | 55220744 | 55220763 | intergenic | 1 | | | 1 | | | | | | | | |
| | (SEQ ID NO: 72) | | 18q21.33 | 59337720 | 59337739 | intergenic | | | | | | | | | | | | |
| sRNA-14 | UGGAGGCAGGAUGACACUGGGA | 22 | 17q21.1 | 35888468 | 35888489 | TNS4, intron (S) | 1 | | | | | | 1 | | | | | |
| | (SEQ ID NO: 73) | | | | | | | | | | | | | | | | | |
| sRNA-15 | UGUGAGAUUGGUGGAAAGGAA | 21 | 3p24.3 | 20069019 | 20069039 | PCAF, intron (S) | 1 | | | | | | | 1 | | | | |
| | (SEQ ID NO: 74) | | | | | | | | | | | | | | | | | |
| sRNA-16 | CUUGGUUUCCCUUUUCCUAUC | 21 | 3q22.1 | 134240115 | 134240135 | TMEM108, intron (S) | 1 | | | | | | | 1 | | | | |
| | (SEQ ID NO: 75) | | | | | | | | | | | | | | | | | |
| sRNA-17 | GCUGGUGAGUGACUGAAUUGA | 21 | 4q25 | 108840979 | 108840999 | PAPSS1, intron (S) | 1 | | | | | | | 1 | | | | |
| | (SEQ ID NO: 76) | | | | | | | | | | | | | | | | | |
| sRNA-18 | CCAGAAACAGAUGGUGGGUAA | 21 | 15q26.3 | 100055065 | 100055085 | TARSL2, intron (AS) | 1 | | | | | | | 1 | | | | |
| | (SEQ ID NO: 77) | | | | | | | | | | | | | | | | | |
| sRNA-19 | CUUCAGUUUCCUUGUGUGCA | 20 | 11q24.2 | 125368777 | 125368796 | CDON, intron (S) | 1 | | | | | | | 1 | | | | |
| | (SEQ ID NO: 78) | | | | | | | | | | | | | | | | | |
| sRNA-20 | CUAUUGGUGGCGCUUGAAUAC | 21 | 12q21.33 | 87654069 | 87654089 | intergenic | 1 | | | | | | | 1 | | | | |
| | (SEQ ID NO: 79) | | | | | | | | | | | | | | | | | |
| sRNA-21 | CAGGUGCUUCCAAGGAGCCAGG | 22 | 17p13.3 | 3446719 | 3446740 | TRPV1, intron (AS) | 1 | | | | | | | 1 | | | | |
| | (SEQ ID NO: 80) | | | | | | | | | | | | | | | | | |
| sRNA-22 | CCACAUCACAACACAUGCAUAG | 22 | 17q25.1 | 69225687 | 69225708 | intergenic | 1 | | | | | | | 1 | | | | |
| | (SEQ ID NO: 81) | | | | | | | | | | | | | | | | | |
| sRNA-23 | CCUCUGAAAUGGAUGGGACCU | 21 | 1p36.11 | 25226518 | 25226538 | intergenic | 1 | | | | | | | 1 | | | | |
| | (SEQ ID NO:82) | | | | | | | | | | | | | | | | | |
| sRNA-24 | CCUAAAAUUGUGAGUGCAUAUCUC | 24 | 5q21.2 | 104049131 | 104049154 | intergenic | 1 | | | | | | | 1 | | | | |
| | (SEQ ID NO: 83) | | | | | | | | | | | | | | | | | |
| sRNA-25 | CCUGAGUGAUGGGCAUGCCUUUGUC | 25 | Xq13.1 | 70875593 | 70875617 | intergenic | 1 | | | | | | | 1 | | | | |
| | (SEQ ID NO: 84) | | | 70879068 | 70879092 | intergenic | | | | | | | | | | | | |
| sRNA-26 | CUAAUCUCUUCUGAUGGGCUCUU | 23 | 15q22.31 | 63436601 | 63436623 | PUNC, intron (AS) | 1 | | | | | | | | | 1 | | |
| | (SEQ ID NO: 85) | | | | | | | | | | | | | | | | | |
| sRNA-27 | CAGUGAAAUUGAUGUGCCUUUGUUU | 25 | 1q43 | 237300018 | 237300042 | intergenic | 1 | | | | | | | | | 1 | | |
| | (SEQ ID NO: 86) | | | | | | | | | | | | | | | | | |
| sRNA-28 | GUAAUAUCAGAAGUUUGGGAGG | 22 | 10q21.2 | 62119258 | 62119279 | ANK3, intron (AS) | 1 | | | | | | | | | | 1 | |
| | (SEQ ID NO: 87) | | | | | | | | | | | | | | | | | |
| sRNA-29 | CGUGGCUAUUUGAUGAGUGUGCAC | 24 | 10q26.3 | 133083670 | 133083693 | intergenic | 1 | | | | | | | | | | 1 | |
| | (SEQ ID NO: 88) | | | | | | | | | | | | | | | | | |
| sRNA-30 | CCCUAGUUGAUGUGGAUGAUUGUC | 24 | 2p21 | 42054979 | 42055002 | intergenic | 1 | | | | | | | | | | 1 | |
| | (SEQ ID NO: 89) | | | | | | | | | | | | | | | | | |
| sRNA-31 | CCCUGUAUUUGGAUGGUUUUC | 21 | 2q31.1 | 174239923 | 174239943 | intergenic | 1 | | | | | | | | | | 1 | |
| | (SEQ ID NO: 90) | | | | | | | | | | | | | | | | | |
| sRNA-32 | CCUCAUGAGGGAGAGUACCCCC | 22 | 5q31.1 | 134512076 | 134512097 | intergenic | 1 | | | | | | | | | | 1 | |
| | (SEQ ID NO: 91) | | | | | | | | | | | | | | | | | |
| sRNA-33 | CCUCAAUAUAGGUGGCUGUAU | 21 | 21q21.2 | 23626072 | 23626092 | intergenic | 1 | | | | | | | | | | | 1 |
| | (SEQ ID NO: 92) | | | | | | | | | | | | | | | | | |

| | | | | | | | | | | | | |
|---|---|---|---|---|---|---|---|---|---|---|---|---|
| Total clones | 37 | 7 | 1 | 4 | 1 | 0 | 5 | 11 | 0 | 2 | 5 | 1 |

^{a}sRNA refers to small RNAs. i
^{b}Sequences listed represent the observed full length sequence of each-sRNA cloned. The extra bases at the termini of some sRNAs are indicated in bold.
^{c}UTR, untranslated region; S, sense; AS, antisense.

TABLE 6

no. of clones

| | | | | | | | | | |
|---|---|---|---|---|---|---|---|---|---|
| | | size | total | | | | | | ME- |
| miRNA | sequence | range | clones | SW756 | C4I | C33A | CaSki | SiHa | 180 |

| | | | | | | | | | |
|---|---|---|---|---|---|---|---|---|---|
| candidate-1 | auauaauacaaccugcuaagugu | 22- | 5 | 4 | | | 1 | | |
| | (SEQ ID NO: 93) | 23 | | | | | | | |
| candidate-2 | ugugcgcagggagaccucuccc | 22 | 1 | 1 | | | | | |
| | (SEQ ID NO: 94) | | | | | | | | |
| candidate-3 | cugggaucuccggggucuuggu | 22 | 1 | 1 | | | | | |
| | (SEQ ID NO: 95) | | | | | | | | |
| candidate-4 | aggaagccuggaggggcuggagg | 24 | 2 | 2 | | | | | |
| | (SEQ ID NO: 96) | | | | | | | | |
| candidate-5 | ugucuacuacuggagacacugg | 22 | 1 | | 1 | | | | |
| | (SEQ ID NO: 97) | | | | | | | | |
| candidate-6 | ccaguuaccgcuuccgcuaccgc | 22- | 2 | | 1 | 1 | | | |
| | (SEQ ID NO: 98) | 23 | | | | | | | |
| candidate-7 | aagggcuuuugggcagguaggug | 24 | 1 | | 1 | | | | |
| | (SEQ ID NO: 99) | | | | | | | | |
| candidate-8 | acaguagagggaggaaucgcag | 22 | 1 | | | | 1 | | |
| | (SEQ ID NO: 100) | | | | | | | | |
| candidate-9 | auccgcgcucugacucucugcc | 22 | 1 | | | | 1 | | |
| | (SEQ ID NO: 101) | | | | | | | | |
| candidate-10 | ugcccuuaaaggugaacccagu | 22 | 1 | | | | | | 1 |
| | (SEQ ID NO: 102) | | | | | | | | |
| candidate-11 | uggggagcugaggcucugggggug | 24 | 1 | | | | | | 1 |
| | (SEQ ID NO: 103) | | | | | | | | |
| candidate-12 | aaggcagggcccccgcucccc | 20- | 2 | | | | | | 2 |
| | (SEQ ID NO: 104) | 21 | | | | | | | |
| candidate-13 | cacccggcugugugcacaugugc | 23 | 1 | | | | | | 1 |
| | (SEQ ID NO: 105) | | | | | | | | |
| candidate-14 | ucuucucuguuuuggccaugug | 22 | 1 | | | | | | 1 |
| | (SEQ ID NO: 106) | | | | | | | | |
| candidate-15 | cugacuguugccguccuccag | 21 | 1 | | | | | | 1 |
| | (SEQ ID NO: 107) | | | | | | | | |
| let-7a | ugagguaguagguuguauaguu | 22 | 231 | 54 | 73 | 10 | 63 | 5 | 26 |
| | (SEQ ID NO: 108) | | | | | | | | |
| let-7b | ugagguaguagguugugugguu | 22 | 318 | 74 | 96 | 4 | 60 | 51 | 33 |
| | (SEQ ID NO: 109) | | | | | | | | |
| let-7b* | cuauacaaccuacugccuucc | 21 | 2 | | 2 | | | | |
| | (SEQ ID NO: 110) | | | | | | | | |
| let-7c | ugagguaguagguuguaugguu | 22 | 21 | | | 18 | 1 | | 2 |
| | (SEQ ID NO: 111) | | | | | | | | |
| let-7d | agagguaguagguugcauagu | 21 | 39 | 6 | 5 | | 22 | | 6 |
| | (SEQ ID NO: 112) | | | | | | | | |
| let-7e | ugagguaggagguuguauagu | 21 | 35 | 9 | 15 | 2 | 7 | | 2 |
| | (SEQ ID NO: 113) | | | | | | | | |
| let-7f | ugagguaguagauuguauaguu | 22 | 145 | 44 | 37 | 5 | 33 | 9 | 17 |
| | (SEQ ID NO: 114) | | | | | | | | |
| let-7g | ugagguaguaguuuguacagu | 21 | 41 | 28 | 8 | 1 | 1 | 2 | 1 |
| | (SEQ ID NO: 115) | | | | | | | | |
| let-7i | ugagguaguaguuugugcugu | 21 | 30 | 13 | 6 | 2 | 6 | 1 | 2 |
| | (SEQ ID NO: 116) | | | | | | | | |
| let-7i* | ugcgcaagcuacugccuugcu | 21 | 2 | 2 | | | | | |
| | (SEQ ID NO: 117) | | | | | | | | |
| miR-7 | uggaagacuagugauuuuguug | 22 | 2 | | 2 | | | | |
| | (SEQ ID NO: 118) | | | | | | | | |
| miR-10a | uacccuguagauccgaauuugug | 23 | 1 | | | | 1 | | |
| | (SEQ ID NO: 119) | | | | | | | | |
| miR-10b | uacccuguagaaccgaauuugu | 22 | 1 | | 1 | | | | |
| | (SEQ ID NO: 120) | | | | | | | | |
| miR-15a | uagcagcacauaaugguuugug | 22 | 4 | 2 | 1 | 1 | | | |
| | (SEQ ID NO: 121) | | | | | | | | |
| miR-15b | uagcagcacaucaugguuuaca | 22 | 18 | 2 | 4 | 3 | 2 | | 7 |
| | (SEQ ID NO: 122 | | | | | | | | |
| miR-15b* | ucaagaugcgaaucauuauuugcugcuccua | 30 | 1 | 1 | | | | | |
| | (SEQ ID NO: 123) | | | | | | | | |
| miR-16 | uagcagcacguaaauauuggcg | 22 | 10 | 13 | 5 | | | 1 | |
| | (SEQ ID NO: 124) | | | | | | | | |
| miR-17-5p | caaagugcuuacagugcagguagu | 24 | 27 | 3 | 12 | | 5 | 2 | 5 |
| | (SEQ ID NO: 125) | | | | | | | | |
| miR-17-3p | acugcagugaaggcacuugu | 20 | 2 | | 2 | | | | |
| | (SEQ ID NO: 126) | | | | | | | | |
| miR-18a | uaaggugcaucuagugcagaua | 22 | 14 | 13 | | | 1 | | |
| | (SEQ ID NO: 127) | | | | | | | | |
| miR-18a* | acugcccuaagugcuccuucug | 22 | 1 | | | | | | 1 |
| | (SEQ ID NO: 128) | | | | | | | | |
| miR-19a | ugugcaaaucuaugcaaaacuga | 23 | 2 | 2 | | | | | |
| | (SEQ ID NO: 129) | | | | | | | | |
| miR-19b | ugugcaaauccaugcaaaacuga | 22 | 3 | | | 2 | | | 1 |
| | (SEQ ID NO: 130) | | | | | | | | |
| mir-20a | uaaagugcuuauagugcagguag | 23 | 25 | 8 | 12 | | | | 5 |
| | (SEQ ID NO: 131) | | | | | | | | |
| miR-20b | caaagugcucauagugcagguag | 23 | 2 | 1 | | | | | 1 |
| | (SEQ ID NO: 132) | | | | | | | | |
| miR-21 | uagcuuaucagacugauguugac | 22- | 956 | 281 | ## | 111 | 196 | 79 | 59 |
| | (SEQ ID NO: 133) | 23 | | | | | | | |
| miR-21* | caacaccagucgaugggcuguc | 22 | 4 | 3 | 1 | | | | |
| | (SEQ ID NO: 134) | | | | | | | | |
| miR-22 | aagcugccaguugaagaacugu | 22 | 1 | 1 | | | | | |
| | (SEQ ID NO: 135) | | | | | | | | |
| miR-23a | aucacauugccagggauuucc | 21 | 126 | 19 | 38 | 3 | 40 | 14 | 12 |
| | (SEQ ID NO: 136) | | | | | | | | |
| miR-23a* | gggguuccuggggaugggauuu | 22 | 2 | | | | 1 | | 1 |
| | (SEQ ID NO: 137) | | | | | | | | |
| miR-23b | aucacauugccagggauuacc | 21 | 20 | 2 | 4 | | 3 | 6 | 5 |
| | (SEQ ID NO: 138) | | | | | | | | |
| miR-23b* | uggguuccuggcaugcugauuu | 22 | 1 | | | | | | 1 |
| | (SEQ ID NO: 139) | | | | | | | | |
| miR-24 | uggcucaguucagcaggaacag | 22 | 20 | 1 | 11 | | 3 | 2 | 3 |
| | (SEQ ID NO: 140) | | | | | | | | |
| miR-25 | cauugcacuugucucggucuga | 22 | 16 | 8 | 2 | | 1 | 2 | 3 |
| | (SEQ ID NO: 141) | | | | | | | | |
| miR-26a | uucaaguaauccaggauaggc | 21 | 23 | 10 | 5 | 5 | 1 | 1 | 1 |
| | (SEQ ID NO: 142) | | | | | | | | |
| miR-26b | uucaaguaauucaggauagguu | 22 | 6 | 1 | 3 | | | 2 | |
| | (SEQ ID NO: 143) | | | | | | | | |
| miR-27a | uucacaguggcuaaguuccgc | 21 | 29 | 6 | 13 | 2 | 3 | 4 | 1 |
| | (SEQ ID NO: 144) | | | | | | | | |
| miR-27b-3p | uucacaguggcuaaguucugc | 21 | 3 | | 1 | | 1 | | 1 |
| | (SEQ ID NO: 145) | | | | | | | | |
| miR-27b-5p | gagcuuagcugauuggugaaca | 22 | 2 | | | | | | 2 |
| | (SEQ ID NO: 146) | | | | | | | | |
| miR-28 | aaggagcucacagucuauugag | 22 | 8 | 7 | | | | | 1 |
| | (SEQ ID NO: 147) | | | | | | | | |
| miR-28* | cacuagauugugagcuccugga | 22 | 2 | | | | | 2 | |
| | (SEQ ID NO: 148) | | | | | | | | |
| miR-29a | uagcaccaucugaaaucgguu | 21 | 34 | 15 | 2 | 1 | 2 | 12 | 2 |
| | (SEQ ID NO: 149) | | | | | | | | |
| miR-29b | uagcaccauuugaaaucaguguu | 23 | 32 | 17 | 4 | | 4 | 5 | 2 |
| | (SEQ ID NO: 150) | | | | | | | | |
| miR-29b* | gcugguuucauauggugguuu | 21 | 4 | 3 | | | 1 | | |
| | (SEQ ID NO: 151) | | | | | | | | |
| miR-29c | uagcaccauuugaaaucggu | 20 | 2 | | 1 | | | | 1 |
| | (SEQ ID NO: 152) | | | | | | | | |
| miR-30a-5p | uguaaacauccucgacuggaag | 22 | 11 | | 5 | | 4 | 1 | 1 |
| | (SEQ ID NO: 153) | | | | | | | | |
| miR-30b | uguaaacauccuacacucagcu | 22 | 11 | 3 | 3 | | 1 | 3 | 1 |
| | (SEQ ID NO: 154) | | | | | | | | |
| miR-30c | uguaaacauccuacacucucagc | 23 | 16 | 3 | 9 | | 2 | | 2 |
| | (SEQ ID NO: 155) | | | | | | | | |
| miR-30d | uguaaacauccccgacuggaag | 22 | 7 | 1 | | | 3 | | 3 |
| | (SEQ ID NO: 156) | | | | | | | | |
| miR-30e-5p | uguaaacauccuugacuggaag | 22 | 2 | | | | | 2 | |
| | (SEQ ID NO: 157) | | | | | | | | |
| miR-31 | ggcaagaugcuggcauagcug | 21 | 12 | | 6 | 1 | | 1 | 4 |
| | (SEQ ID NO: 158) | | | | | | | | |
| miR-34a | uggcagugucuuagcugguuguu | 23 | 5 | | 1 | | 1 | | 3 |
| | (SEQ ID NO: 159) | | | | | | | | |
| miR-92 | uauugcacuugucccggccug | 21 | 28 | 3 | 5 | 5 | 5 | 7 | 3 |
| | (SEQ ID NO: 160) | | | | | | | | |
| miR-92* | agguugggaucgguugcaaugcu | 23 | 1 | | 1 | | | | |
| | (SEQ ID NO: 161) | | | | | | | | |
| miR-92b | uauugcacucgucccggccucc | 19- | 13 | 3 | 2 | 7 | | 1 | |
| | (SEQ ID NO: 162) | 22 | | | | | | | |
| miR-93 | aaagugcuguucgugcagguag | 22 | 29 | 11 | 4 | 2 | 5 | 4 | 3 |
| | (SEQ ID NO: 163) | | | | | | | | |
| miR-96 | uuuggcacuagcacauuuuugcu | 23 | 2 | | | | 2 | | |
| | (SEQ ID NO: 164) | | | | | | | | |
| miR-98 | ugagguaguaaguuguauuguu | 22 | 13 | 6 | 2 | | 3 | | 2 |
| | (SEQ ID NO: 165) | | | | | | | | |
| miR-99a | aacccguagauccgaucuugug | 22 | 5 | | | 5 | | | |
| | (SEQ ID NO: 166) | | | | | | | | |
| miR-99b | cacccguagaaccgaccuugcg | 22 | 15 | 4 | 3 | 1 | | 4 | 3 |
| | (SEQ ID NO: 167) | | | | | | | | |
| miR-100 | aacccguagauccgaacuugug | 22 | 15 | 5 | 3 | | | | 7 |
| | (SEQ ID NO: 168) | | | | | | | | |
| miR-101 | uacaguacugugauaacugaag | 22 | 5 | 4 | 1 | | | | |
| | (SEQ ID NO: 169) | | | | | | | | |
| miR-101* | caguuaucacagugcugaugcugu | 24 | 1 | 1 | | | | | |
| | (SEQ ID NO: 170) | | | | | | | | |
| miR-103 | agcagcauuguacagggcuauga | 23 | 21 | 5 | 5 | | 3 | | 8 |
| | (SEQ ID NO: 171) | | | | | | | | |
| miR-106b | uaaagugcugacagugcagau | 21 | 16 | 10 | 4 | 2 | | | |
| | (SEQ ID NO: 172) | | | | | | | | |
| miR-106b* | ccgcacuguggguacuugcu | 20 | 4 | 3 | | | 1 | | |
| | (SEQ ID NO: 173) | | | | | | | | |
| miR-125a | ucccugagacccuuuaaccugug | 23 | 70 | 5 | 31 | 1 | 10 | 19 | 4 |
| | (SEQ ID NO: 174) | | | | | | | | |
| miR-125a* | acaggugagguucuugggagcc | 21- | 4 | | 3 | | | | 1 |
| | (SEQ ID NO: 175) | 22 | | | | | | | |
| miR-125b | ucccugagacccuaacuuguga | 22 | 50 | 17 | 3 | 20 | 1 | 4 | 5 |
| | (SEQ ID NO: 176) | | | | | | | | |
| miR-130a | cagugcaauguuaaaagggcau | 22 | 25 | 5 | 3 | 2 | 6 | | 9 |
| | (SEQ ID NO: 177) | | | | | | | | |
| miR-130b | cagugcaaugaugaaagggcau | 22 | 3 | | 3 | | | | |
| | (SEQ ID NO: 178) | | | | | | | | |
| miR-130b* | acucuuucccuguugcacaucu | 22 | 1 | | | | | | 1 |
| | (SEQ ID NO: 179) | | | | | | | | |
| miR-132 | uaacagucuacagccauggucg | 22 | 1 | | 1 | | | | |
| | (SEQ ID NO: 180) | | | | | | | | |
| miR-135b | uauggcuuuucauuccuaugug | 22 | 1 | | | | 1 | | |
| | (SEQ ID NO: 181) | | | | | | | | |
| miR-138 | agcugguguugugaaucaggcc | 22 | 1 | | | | 1 | | |
| | (SEQ ID NO: 182) | | | | | | | | |
| miR-140* | accacaggguagaaccacgga | 21 | 2 | 2 | | | | | |
| | (SEQ ID NO: 183) | | | | | | | | |
| miR-141 | uaacacugucugguaaagauggc | 23 | 1 | | | | 1 | | |
| | (SEQ ID NO: 184) | | | | | | | | |
| miR-148b | ucagugcaucacagaacuuugu | 22 | 2 | 1 | 1 | | | | |
| | (SEQ ID NO: 185) | | | | | | | | |
| miR-149-5p | ucuggcuccgugucuucacucc | 22 | 2 | | 2 | | | | |
| | (SEQ ID NO: 186) | | | | | | | | |
| miR-149-3p | agggagggacgggggcugugc | 21 | 1 | | 1 | | | | |
| | (SEQ ID NO: 187) | | | | | | | | |
| miR-151-3p | acuagacugaagcuccuugagg | 22 | 1 | 1 | | | | | |
| | (SEQ ID NO: 188) | | | | | | | | |
| miR-151-5p | ucgaggagcucacagucuagua | 22 | 93 | 11 | 15 | 8 | 20 | 24 | 15 |
| | (SEQ ID NO: 189) | | | | | | | | |
| miR-152 | ucagugcaugacagaacuuggg | 22 | 1 | | | | 1 | | |
| | (SEQ ID NO: 190) | | | | | | | | |
| miR-155 | uuaaugcuaaucgugauagggg | 22 | 4 | 4 | | | | | |
| | (SEQ ID NO: 191) | | | | | | | | |
| miR-181a | aacauucaacgcugucggugagu | 23 | 5 | | | 2 | 1 | | 2 |
| | (SEQ ID NO: 192) | | | | | | | | |
| miR-181b | aacauucauugcugucgguggg | 22 | 9 | 2 | 1 | | 4 | 1 | 1 |
| | (SEQ ID NO: 193) | | | | | | | | |
| miR-182 | uuuggcaaugguagaacucaca | 22 | 9 | 1 | 1 | | 5 | 2 | |
| | (SEQ ID NO: 194) | | | | | | | | |
| miR-183 | uauggcacugguagaauucacug | 23 | 8 | 4 | 3 | | | | 1 |
| | (SEQ ID NO: 195) | | | | | | | | |
| miR-185 | uggagagaaaggcaguuccugau | 23 | 14 | 1 | | 1 | 1 | 2 | 9 |
| | (SEQ ID NO: 196) | | | | | | | | |
| miR-186 | caaagaauucuccuuuugggcuu | 23 | 4 | 1 | 1 | | | | 2 |
| | (SEQ ID NO: 197) | | | | | | | | |
| miR-191 | caacggaaucccaaaagcagcu | 22 | 32 | 5 | 5 | 7 | 4 | | 11 |
| | (SEQ ID NO: 198) | | | | | | | | |
| miR-193a | aacuggccuacaaagucccag | 23 | 4 | 2 | | 1 | | | 1 |
| | (SEQ ID NO: 199) | | | | | | | | |
| miR-193a* | ugggucuuugcgggcgagauga | 22 | 1 | | | | | | 1 |
| | (SEQ ID NO: 200) | | | | | | | | |
| miR-193b | aacuggcccucaaagucccgcuuu | 24 | 10 | | | 2 | 7 | | 1 |
| | (SEQ ID NO: 201) | | | | | | | | |
| miR-193b* | cgggguuuugagggcgagauga | 22 | 4 | | | | 2 | 2 | |
| | (SEQ ID NO: 202) | | | | | | | | |
| miR-196a | uagguaguuucauguuguugg | 21 | 2 | 1 | 1 | | | | |
| | (SEQ ID NO: 203) | | | | | | | | |
| miR-196b | uagguaguuuccuguuguugg | 21 | 3 | 1 | 2 | | | | |
| | (SEQ ID NO: 204) | | | | | | | | |
| miR-197 | uucaccaccuucuccacccagc | 22 | 1 | | | 1 | | | |
| | (SEQ ID NO: 205) | | | | | | | | |
| miR-200a | uaacacugucugguaacgaugu | 22 | 5 | | 1 | | | | 4 |
| | (SEQ ID NO: 206) | | | | | | | | |
| miR-200b | uaauacugccugguaaugaugac | 23 | 12 | | 5 | | 5 | | 2 |
| | (SEQ ID NO: 207) | | | | | | | | |
| miR-200c | uaauacugccggguaaugaugg | 22 | 143 | 4 | 17 | | 16 | 3 | 103 |
| | (SEQ ID NO: 208) | | | | | | | | |
| miR-205 | uccuucauuccaccggagucug | 22 | 75 | 6 | 30 | | 22 | | 17 |
| | (SEQ ID NO: 209) | | | | | | | | |
| miR-210 | cugugcgugugacagcggcuga | 22 | 6 | 1 | 1 | | 3 | | 1 |
| | (SEQ ID NO: 210) | | | | | | | | |
| miR-218 | uugugcuugaucuaaccaugu | 21 | 2 | | | 2 | | | |
| | (SEQ ID NO: 211) | | | | | | | | |
| miR-221 | agcuacauugucugcuggguuuc | 23 | 9 | 4 | 2 | | 1 | | 2 |
| | (SEQ ID NO: 212) | | | | | | | | |
| miR-222 | agcuacaucuggcuacugggucuc | 24 | 6 | 4 | | | 2 | | |
| | (SEQ ID NO: 213) | | | | | | | | |
| miR-224 | caagucacuagugguuccguuuag | 24 | 4 | 3 | | | | | 1 |
| | (SEQ ID NO: 214) | | | | | | | | |
| miR-296 | agggcccccccucaauccugu | 21 | 1 | | 1 | | | | |
| | (SEQ ID NO: 215) | | | | | | | | |
| miR-320 | aaaagcuggguugagagggcgaa | 23 | 26 | 7 | 5 | 3 | 5 | 1 | 5 |
| | (SEQ ID NO: 216) | | | | | | | | |
| miR-324-5p | cgcauccccuagggcauuggugu | 23 | 2 | | | | 2 | | |
| | (SEQ ID NO: 217) | | | | | | | | |
| miR-324-3p | ccacugccccaggugcugcugg | 22 | 1 | 1 | | | | | |
| | (SEQ ID NO: 218) | | | | | | | | |
| miR-330* | ucucugggccugugucuua | 19 | 1 | | 1 | | | | |
| | (SEQ ID NO: 219) | | | | | | | | |
| miR-331 | gccccugggccuauccuagaa | 21 | 4 | 3 | | | 1 | | |
| | (SEQ ID NO: 220) | | | | | | | | |
| miR-335 | ucaagagcaauaacgaaaaaugu | 23 | 1 | | 1 | | | | |
| | (SEQ ID NO: 221) | | | | | | | | |
| miR-338* | aacaauauccuggugcugag | 20 | 1 | | | | | | 1 |
| | (SEQ ID NO: 222) | | | | | | | | |
| miR-342 | ucucacacagaaaucgcacccguc | 24 | 6 | | 1 | | | | 5 |
| | (SEQ ID NO: 223) | | | | | | | | |
| miR-342* | aggggugcuaucugugauugagg | 23 | 2 | | | | | | 2 |
| | (SEQ ID NO: 224) | | | | | | | | |
| miR-365 | uaaugccccuaaaaauccuuau | 22 | 4 | | 1 | | 2 | | 1 |
| | (SEQ ID NO: 225) | | | | | | | | |
| miR-374 | uuauaauacaaccugauaagug | 22 | 1 | | 1 | | | | |
| | (SEQ ID NO: 226) | | | | | | | | |
| miR-423-5p | ugaggggcagagagcgagacuuu | 23 | 4 | 1 | | | 1 | | 2 |
| | (SEQ ID NO: 227) | | | | | | | | |
| miR-424-3p | caaaacgugaggcgcugcuau | 21 | 8 | | | | 1 | 4 | 3 |
| | (SEQ ID NO: 228) | | | | | | | | |
| miR-425-5p | aaugacacgaucacucccguug | 22 | 2 | | | | 1 | | 1 |
| | (SEQ ID NO: 229) | | | | | | | | |
| miR-429 | uaauacugucugguaaaaccgu | 22 | 1 | | | | | | 1 |
| | (SEQ ID NO: 230) | | | | | | | | |
| miR-452 | uguuugcagaggaaacugagac | 23 | 3 | | 1 | | | | 2 |
| | (SEQ ID NO: 231) | | | | | | | | |
| miR-455-5p | uaugugccuuuggacuacaucg | 22 | 1 | | | | 1 | | |
| | (SEQ ID NO: 232) | | | | | | | | |
| miR-455-3p | augcaguccaugggcauauacac | 23 | 3 | | | | | 3 | |
| | (SEQ ID NO: 233) | | | | | | | | |
| miR-484 | ucaggcucaguccccucccgau | 22 | 1 | | | | | | 1 |
| | (SEQ ID NO: 234) | | | | | | | | |
| miR-491 | aguggggaacccuuccaugagga | 23 | 1 | | 1 | | | | |
| | (SEQ ID NO: 235) | | | | | | | | |
| miR-505 | gucaacacuugcugguuuccucu | 22 | 2 | 1 | | | | | 1 |
| | (SEQ ID NO: 236) | | | | | | | | |
| miR-522 | aaaaugguucccuuuagaguguu | 23 | 1 | | 1 | | | | |
| | (SEQ ID NO: 237) | | | | | | | | |
| miR-532 | caugccuugaguguaggaccgu | 22 | 4 | | | 3 | 1 | | |
| | (SEQ ID NO: 238) | | | | | | | | |
| miR-542-5p | cggggaucaucaugucacgaga | 22 | 1 | | 1 | | | | |
| | (SEQ ID NO: 239) | | | | | | | | |
| miR-574 | cacgcucaugcacacacccaca | 20- | 7 | 5 | | 2 | | | |
| | (SEQ ID NO: 240) | 22 | | | | | | | |
| miR-582-3p | uaacugguugaacaacugaac | 21 | 1 | | | | | | 1 |
| | (SEQ ID NO: 241) | | | | | | | | |
| miR-584 | uuaugguuugccugggacuga | 21 | 1 | | 1 | | | | |
| | (SEQ ID NO: 242) | | | | | | | | |
| total clones | | | 3253 | 823 | 802 | 249 | 620 | 288 | 471 |

miR-374b to miR-943 are novel miRNAs identified in this study.
The asterisk (*) refers to the sequences paired with predominantly cloned mature miRNAs.
The indices 5p and 3p (indicating the 5′ or 3′ positioned arm of the pre-miRNA) are assigned if the cloning frequency of the two strands of a processed pre-miRNA was comparable. miRNAs that have not been annotated in miR Base are in italic. miRNAs that were computationally predicted but have not been experimentally verified in human are underlined. The extra bases at the termini of some miRNAs are denoted in bold.

For normal cervical samples, a total of 706 (32%) clones were annotated as previously identified miRNAs (Table 7): 67 previously verified miRNAs, 15 computationally predicted miRNAs but never verified experimentally and 6 sequences paired with known mature miRNAs that have not been annotated in miRBase. The remaining small RNAs correspond to fragments of rRNA (22%), tRNA (36%), scRNA/snRNA (2%), repeat sequences (2%), mitochondrial (2%), mRNA (1%) and not mapped/unknown (3%) (FIG. 4B, Table 5). The higher fractions of fragments from degradation of rRNA and tRNA-derived fragments in the clinical samples appear likely to represent general damage or dying cells in the clinical samples; rRNA and tRNA derived fragments were not incorporated in the miRNA profile analysis. A total of 30 clones (1%) could be suggested as novel small RNAs, based on the criteria described above. Of these, five could be annotated as novel miRNAs (Table 1), five are miRNA-like molecules with non-canonical hairpin (Table 2), and 20 are without significant hairpin (Table 3).

TABLE 7

| | | |
|---|---|---|
| size | total | no. of clones |

| | | | | | | | | |
|---|---|---|---|---|---|---|---|---|
| miRNA | sequence | range | clones | NC1 | NC2 | NC3 | NC4 | NC5 |

| | | | | | | | | |
|---|---|---|---|---|---|---|---|---|
| Candidate-16 | aaauuauuguacaucggaugag | 22 | 1 | 1 | | | | |
| | (SEQ ID NO: 243) | | | | | | | |
| Candidate-17 | aaggagcuuacaaucuagcugg | 22 | 2 | | 2 | | | |
| | (SEQ ID NO: 244) | | | | | | | |
| Candidate-18-5p | cggccccacgcaccaggguaag | 22 | 1 | | 1 | | | |
| | (SEQ ID NO: 245) | | | | | | | |
| Candidate-18-3p | cugcccuggcccgagggaccg | 21 | 1 | | | | 1 | |
| | (SEQ ID NO: 246) | | | | | | | |
| let-7a | ugagguaguagguuguauaguu | 22 | 57 | 18 | 12 | 9 | 14 | 4 |
| | (SEQ ID NO: 247) | | | | | | | |
| let-7b | ugagguaguagguugugugguu | 22 | 124 | 33 | 25 | 13 | 18 | 35 |
| | (SEQ ID NO: 248) | | | | | | | |
| let-7c | ugagguaguagguuguaugguu | 22 | 113 | 28 | 27 | 13 | 21 | 24 |
| | (SEQ ID NO: 249) | | | | | | | |
| let-7d | agagguaguagguugcauagu | 21 | 6 | 1 | 2 | 1 | 1 | 1 |
| | (SEQ ID NO: 250) | | | | | | | |
| let-7e | ugagguaggagguuguauagu | 21 | 3 | 1 | | | 1 | 1 |
| | (SEQ ID NO: 251) | | | | | | | |
| let-7f | ugagguaguagauuguauaguu | 22 | 28 | 13 | 4 | 6 | 2 | 3 |
| | (SEQ ID NO: 252) | | | | | | | |
| let-7g | ugagguaguaguuuguacagu | 21 | 7 | 1 | 2 | 3 | 1 | |
| | (SEQ ID NO: 253) | | | | | | | |
| let-7i | ugagguaguaguuugugcugu | 21 | 5 | 3 | | | 2 | |
| | (SEQ ID NO: 254) | | | | | | | |
| miR-15b | uagcagcacaucaugguuuaca | 22 | 2 | | 1 | | 1 | |
| | (SEQ ID NO: 255) | | | | | | | |
| miR-16 | uagcagcacguaaauauuggcg | 22 | 1 | | | | | 1 |
| | (SEQ ID NO: 256) | | | | | | | |
| miR-17-5p | caaagugcuuacagugcagguagu | 24 | 6 | 1 | 5 | | | |
| | (SEQ ID NO: 257) | | | | | | | |
| miR-18a | uaaggugcaucuagugcagaua | 22 | 1 | 1 | | | | |
| | (SEQ ID NO: 258) | | | | | | | |
| miR-21 | uagcuuaucagacugauguugac | 22- | 52 | 6 | 9 | 5 | 13 | 19 |
| | (SEQ ID NO: 259) | 23 | | | | | | |
| miR-21* | caacaccagucgaugggcuguc | 22 | 2 | | | 2 | | |
| | (SEQ ID NO: 260) | | | | | | | |
| miR-22 | aagcugccaguugaagaacugu | 22 | 2 | 2 | | | | |
| | (SEQ ID NO: 261) | | | | | | | |
| miR-23a | aucacauugccagggauuucca | 22 | 15 | 5 | 1 | 3 | 5 | 1 |
| | (SEQ ID NO: 262) | | | | | | | |
| miR-23b | aucacauugccagggauuaccac | 21- | 18 | 10 | 1 | | 4 | 3 |
| | (SEQ ID NO: 263) | 23 | | | | | | |
| miR-25 | cauugcacuugucucggucuga | 22 | 5 | | | | | 5 |
| | (SEQ ID NO: 264) | | | | | | | |
| miR-26a | uucaaguaauccaggauaggcu | 21- | 9 | 2 | 5 | 1 | | 1 |
| | (SEQ ID NO: 265) | 22 | | | | | | |
| miR-26b | uucaaguaauucaggauagguug | 22- | 2 | 2 | | | | |
| | (SEQ ID NO: 266) | 23 | | | | | | |
| miR-27a | uucacaguggcuaaguuccgc | 21 | 4 | 1 | 2 | | | 1 |
| | (SEQ ID NO: 267) | | | | | | | |
| miR-27b-3p | uucacaguggcuaaguucugca | 21- | 2 | 1 | | 1 | | |
| | (SEQ ID NO: 268) | 22 | | | | | | |
| miR-29a | uagcaccaucugaaaucgguuau | 21- | 6 | | 2 | 1 | | 3 |
| | (SEQ ID NO: 269) | 23 | | | | | | |
| miR-29b | uagcaccauuugaaaucaguguu | 23 | 3 | 1 | | | | 2 |
| | (SEQ ID NO: 270) | | | | | | | |
| miR-30a-3p | cuuucagucggauguuugcagc | 22 | 1 | | | | | 1 |
| | (SEQ ID NO: 271) | | | | | | | |
| miR-31 | ggcaagaugcuggcauagcug | 21 | 1 | | 1 | | | |
| | (SEQ ID NO: 272) | | | | | | | |
| miR-33 | gugcauugauguugcauugc | 20 | 1 | | | | 1 | |
| | (SEQ ID NO: 273) | | | | | | | |
| miR-34a | uggcagugucuuagcugguuguu | 23 | 1 | | | | | 1 |
| | (SEQ ID NO: 274) | | | | | | | |
| miR-92 | uauugcacuugucccggccugu | 22 | 14 | 1 | | 3 | 2 | 8 |
| | (SEQ ID NO: 275) | | | | | | | |
| miR-98 | ugagguaguaaguuguauuguu | 22 | 1 | 1 | | | | |
| | (SEQ ID NO: 276) | | | | | | | |
| miR-99a | aacccguagauccgaucuugug | 22 | 6 | 1 | 1 | 1 | 3 | |
| | (SEQ ID NO: 277) | | | | | | | |
| miR-99b | cacccguagaaccgaccuugcg | 22 | 5 | 1 | | | 1 | 3 |
| | (SEQ ID NO: 278) | | | | | | | |
| miR-100 | aacccguagauccgaacuugug | 22 | 6 | 3 | | 1 | 1 | 1 |
| | (SEQ ID NO: 279) | | | | | | | |
| miR-124a | uuaaggcacgcggugaaugcca | 22 | 1 | 1 | | | | |
| | (SEQ ID NO: 280) | | | | | | | |
| miR-125a | ucccugagacccuuuaaccugug | 23 | 8 | 1 | 4 | | 3 | |
| | (SEQ ID NO: 281) | | | | | | | |
| miR-125b | ucccugagacccuaacuuguga | 22 | 18 | 3 | 7 | 5 | 2 | 1 |
| | (SEQ ID NO: 282) | | | | | | | |
| miR-126 | ucguaccgugaguaauaaugcg | 22 | 3 | | 1 | 1 | 1 | |
| | (SEQ ID NO: 283) | | | | | | | |
| miR-127 | ucggauccgucugagcuuggcu | 22- | 1 | 1 | | | | |
| | (SEQ ID NO: 284) | 23 | | | | | | |
| miR-128b | ucacagugaaccggucucuuu | 21 | 1 | | | | 1 | |
| | (SEQ ID NO: 285) | | | | | | | |
| miR-133a | uugguccccuucaaccagcugu | 22 | 1 | | | 1 | | |
| | (SEQ ID NO: 286) | | | | | | | |
| miR-140* | uaccacaggguagaaccacgg | 21 | 1 | | | | | 1 |
| | (SEQ ID NO: 287) | | | | | | | |
| miR-142-3p | uguaguguuuccuacuuuaugga | 23 | 2 | 1 | | | 1 | |
| | (SEQ ID NO: 288) | | | | | | | |
| miR-143 | ugagaugaagcacuguagcuca | 21- | 7 | 1 | 3 | | 2 | 1 |
| | (SEQ ID NO: 289) | 22 | | | | | | |
| miR-145 | guccaguuuucccaggaaucccu | 23 | 2 | 1 | 1 | | | |
| | (SEQ ID NO: 290) | | | | | | | |
| miR-146a | ugagaacugaauuccauggguu | 22 | 1 | | | | | 1 |
| | (SEQ ID NO: 291) | | | | | | | |
| miR-148b | ucagugcaucacagaacuuugu | 22 | 1 | | | | | 1 |
| | (SEQ ID NO: 292) | | | | | | | |
| miR-151-5p | ucgaggagcucacagucuagua | 22 | 22 | 4 | 9 | | 1 | 8 |
| | (SEQ ID NO: 293) | | | | | | | |
| miR-151-3p | acuagacugaagcuccuugagg | 22 | 1 | 1 | | | | |
| | (SEQ ID NO: 294) | | | | | | | |
| miR-154 | uagguuauccguguugccuucg | 22 | 2 | 1 | 1 | | | |
| | (SEQ ID NO: 295) | | | | | | | |
| miR-155 | uuaaugcuaaucgugauagggg | 22 | 1 | | 1 | | | |
| | (SEQ ID NO: 296) | | | | | | | |
| miR-181a* | accaucgaccguugauuguacc | 22 | 1 | | | | | 1 |
| | (SEQ ID NO: 297) | | | | | | | |
| miR-181b | aacauucauugcugucgguggg | 22 | 1 | | | | 1 | |
| | (SEQ ID NO: 298) | | | | | | | |
| miR-181d | aacauucauuguugucgguggg | 22 | 1 | | 1 | | | |
| | (SEQ ID NO: 299) | | | | | | | |
| miR-182 | uuuggcaaugguagaacucaca | 22 | 1 | 1 | | | | |
| | (SEQ ID NO: 300) | | | | | | | |
| miR-185 | uggagagaaaggcaguuccugau | 23 | 3 | | 1 | | | 2 |
| | (SEQ ID NO: 301) | | | | | | | |
| miR-191 | caacggaaucccaaaagcagcu | 22 | 3 | | | | 2 | 1 |
| | (SEQ ID NO: 302) | | | | | | | |
| miR-196b | uagguaguuuccuguuguugg | 21 | 9 | 4 | 1 | 2 | 1 | 1 |
| | (SEQ ID NO: 303) | | | | | | | |
| miR-199a | cccaguguucagacuaccuguuc | 22- | 2 | | 1 | | 1 | |
| | (SEQ ID NO: 304) | 23 | | | | | | |
| miR-199b | cccaguguuuagacuaucuguuca | 24 | 1 | | 1 | | | |
| | (SEQ ID NO: 305) | | | | | | | |
| miR-200a | uaacacugucugguaacgaugu | 22 | 1 | | | | 1 | |
| | (SEQ ID NO: 306) | | | | | | | |
| miR-200b | uaauacugccugguaaugaugac | 23 | 1 | 1 | | | | |
| | (SEQ ID NO: 307) | | | | | | | |
| miR-200c | uaauacugccggguaaugaugg | 22 | 57 | 12 | 8 | 8 | 18 | 11 |
| | (SEQ ID NO: 308) | | | | | | | |
| miR-205 | uccuucauuccaccggagucug | 22 | 10 | 4 | 1 | 2 | 1 | 2 |
| | (SEQ ID NO: 309) | | | | | | | |
| miR-210 | cugugcgugugacagcggcuga | 22 | 1 | | | | | 1 |
| | (SEQ ID NO: 310) | | | | | | | |
| miR-214 | acagcaggcacagacaggca | 20 | 1 | | | | 1 | |
| | (SEQ ID NO: 311) | | | | | | | |
| miR-219 | ugauuguccaaacgcaauucu | 21 | 1 | 1 | | | | |
| | (SEQ ID NO: 312) | | | | | | | |
| miR-222 | agcuacaucuggcuacugggucuc | 24 | 1 | | | | 1 | |
| | (SEQ ID NO: 313) | | | | | | | |
| miR-299-3p | uaugugggaugguaaaccgcu | 21 | 1 | | 1 | | | |
| | (SEQ ID NO: 314) | | | | | | | |
| miR-320 | aaaagcuggguugagagggcgaa | 23 | 4 | 1 | | 2 | | 1 |
| | (SEQ ID NO: 315) | | | | | | | |
| miR-324-5p | cgcauccccuagggcauuggugu | 23 | 1 | | 1 | | | |
| | (SEQ ID NO: 316) | | | | | | | |
| miR-328 | cuggcccucucugcccuuccgu | 22 | 1 | | 1 | | | |
| | (SEQ ID NO: 317) | | | | | | | |
| miR-365 | uaaugccccuaaaaauccuuau | 22 | 1 | | | 1 | | |
| | (SEQ ID NO: 318) | | | | | | | |
| miR-368 | aacauagaggaaauuccacgu | 21 | 1 | | | 1 | | |
| | (SEQ ID NO: 319) | | | | | | | |
| miR-374 | uuauaauacaaccugauaagug | 22 | 3 | 1 | | 1 | 1 | |
| | (SEQ ID NO: 320) | | | | | | | |
| miR-375 | uuuguucguucggcucgcguga | 22 | 1 | | 1 | | | |
| | (SEQ ID NO: 321) | | | | | | | |
| miR-376a | aucauagaggaaaauccacgu | 21 | 1 | | 1 | | | |
| | (SEQ ID NO: 322) | | | | | | | |
| miR-381 | uauacaagggcaagcucucugu | 22 | 1 | | | | 1 | |
| | (SEQ ID NO: 323) | | | | | | | |
| miR-423-5p | ugaggggcagagagcgagacuuu | 23 | 1 | 1 | | | | |
| | (SEQ ID NO: 324) | | | | | | | |
| miR-424-5p | cagcagcaauucauguuuuga | 21 | 1 | | | 1 | | |
| | (SEQ ID NO: 325) | | | | | | | |
| miR-424-3p | caaaacgugaggcgcugcuau | 21 | 1 | | | | | 1 |
| | (SEQ ID NO: 326) | | | | | | | |
| miR-425-5p | aaugacacgaucacucccguug | 22 | 1 | | | | | 1 |
| | (SEQ ID NO: 327) | | | | | | | |
| miR-450 | uuuugcgauguguuccuaau | 20 | 1 | 1 | | | | |
| | (SEQ ID NO: 328) | | | | | | | |
| miR-455-3p | augcaguccaugggcauauacac | 23 | 2 | 2 | | | | |
| | (SEQ ID NO: 329) | | | | | | | |
| miR-494 | ugaaacauacacgggaaaccucu | 23 | 1 | | | | 1 | |
| | (SEQ ID NO: 330) | | | | | | | |
| miR-495 | aaacaaacauggugcacuucuu | 22 | 2 | 1 | | | 1 | |
| | (SEQ ID NO: 331) | | | | | | | |
| miR-505 | gucaacacuugcugguuuccucu | 22 | 1 | | 1 | | | |
| | (SEQ ID NO: 332) | | | | | | | |
| miR-542-3p | ugugacagauugauaacuga | 20 | 1 | 1 | | | | |
| | (SEQ ID NO: 333) | | | | | | | |
| miR-574 | cacgcucaugcacacacccaca | 22 | 2 | | 1 | | | 1 |
| | (SEQ ID NO: 334) | | | | | | | |
| Total clones | | | 711 | 184 | 150 | 89 | 133 | 155 |

miR-943 to miR-874-3p are novel miRNAs identified in this study.
The asterisk (*) refers to the sequences paired with predominantly cloned mature miRNAs.
The indices 5p and 3p (indicating the 5′ or 3′ positioned arm of the pre-miRNA) are assigned if the cloning frequency of the two strands of a processed pre-miRNA was comparable. miRNAs that have not been annotated in miRBase are in italic. miRNAs that were computationally predicted but have not been experimentally verified in human are underlined. The extra bases at the termini of some miRNAs are denoted in bold.
NC1-5 are five different histopathologically verified normal cervical samples.

Identification of novel miRNAs. Seventeen new miRNAs were identified from 26 relevant clones, in which 14 were found in cervical cancer cell lines and three were found in normal cervical samples (Table 1). Five of these candidates were observed more than once and two were found in more than one cell line (Tables 1 and 6-7). As expected, the size of these novel miRNAs is in the range of 20-24 nucleotides (nt), with a mode of 22 nt. Of the 17 novel miRNAs, 15 are found in other mammalian genomes (but not in invertebrates), while two candidates (miR-933 and miR-769-3p) appeared not to be conserved.

Although the 17 novel candidate miRNAs are all unique in sequence, some sequences are similar to previously identified human miRNAs: miR-374b is very similar to miR-374 and miR-708 is similar to miR-28. In addition, the two strands of a novel candidate miRNA, miR-874, were independently identified in different normal cervical samples.

Identification of small RNAs with non-canonical hairpin. Other than the novel candidate miRNAs, we identified nine small RNAs (from 12 clones) that are perfectly matched to the human genome but do not fully meet the miRNA criteria as described above. We designate these small RNAs (four from cervical cancer cell lines and five from normal cervices) as sRNA-cer (i.e. small RNAs in cervix). Two of these sRNAs were observed more than once (Table 2). Six sRNAs (sRNA-cer2, 3, 5, 6, 7 and 9) are located in the intergenic regions, while three sRNAs are found in the intron or exon of known genes. sRNA-cert is located in the exon 9 (NM_{—}003879) or intron 7 (AF009619) of CFLAR (CASP8 and FADD-like apoptosis regulator). sRNA-cer4 overlaps with intron 1 of WDR20 (WD repeat domain 20) and transcribed from the opposite DNA strand. Similarly, sRNA-cer8 is the antisense strand that overlaps with intron 1 of SCARA3 (Scavenger receptor class A, member 3). Notably, most of these sRNAs are not conserved beyond primates.

Identification of small RNAs without significant hairpin. We also identified 33 small RNAs from a total of 37 clones that are perfectly matched to the human genome but do not have a significant hairpin structure (Table 3). These sRNAs are 18-25 nt in size, with a predominant class in 21 nt. Fourteen of them were found in cervical cancer cell lines, while 19 were identified in normal cervical samples. Only four of these sRNAs were observed more than once.

These sRNAs map to different loci in 20 different human chromosomes; only two (sRNA-6 and -7) are located at the same chromosome region 5q31.2 (56 by apart). Notably, sRNA-6 and -7 match the sense strand of a predicted Ensembl transcript, ENST00000365160 (http://www.ensembl.org/). Interestingly, this sequence is predicted to have a consensus secondary structure for vault RNA family (FIG. S2). Vault RNAs are found as part of the vault ribonucleoprotein complex that has been suggested to play a role in drug resistance. The two sRNA sequences identified are paired in the stem-loop structure (FIG. 5).

In this class of sRNAs, 19 are located in putative intergenic regions, while 14 are in known protein-coding segments. Among the sRNAs that correspond to protein-coding sequences, eight are sense to the intron and six are antisense to the coding-strand of mRNAs (4 in the intron, 1 in the exon and 1 in the 3′UTR). Notably, one of the intronic sRNA, sRNA-5, is located upstream of miR-33 and in the same intron of SREBF2. The two exonic antisense sRNAs include: (i) sRNA-11, which is antisense to exon 16 of BAT2D1 (BAT2 domain containing 1), and (ii) sRNA-12, which is antisense to the 3′UTR of MPL (myeloproliferative leukemia virus oncogene).

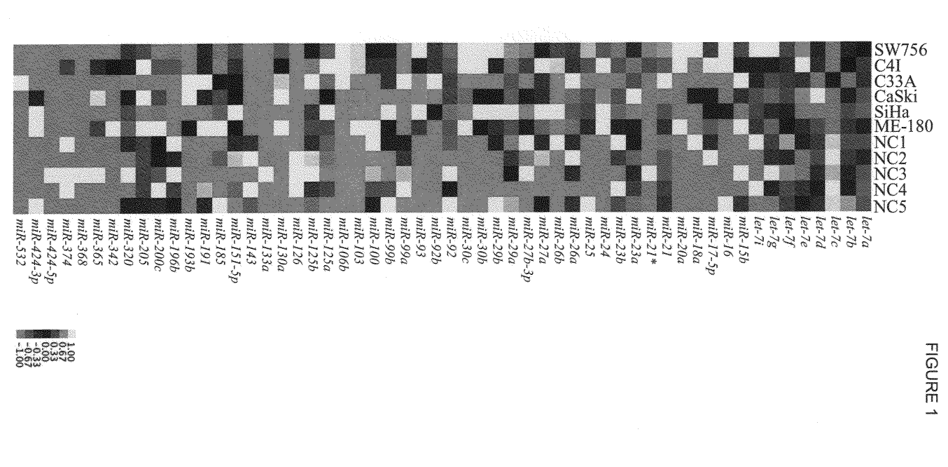

miRNA expression profiles in human cervical cancer cell lines and normal cervix. To assess the relative abundance level of each miRNA between samples, we compared the relative cloning incidence with respect to the total number of small RNA clones. As shown in FIG. 1, miRNA expression patterns were generally similar among the cell lines and among normal cervices, although some variations were observed within each group.

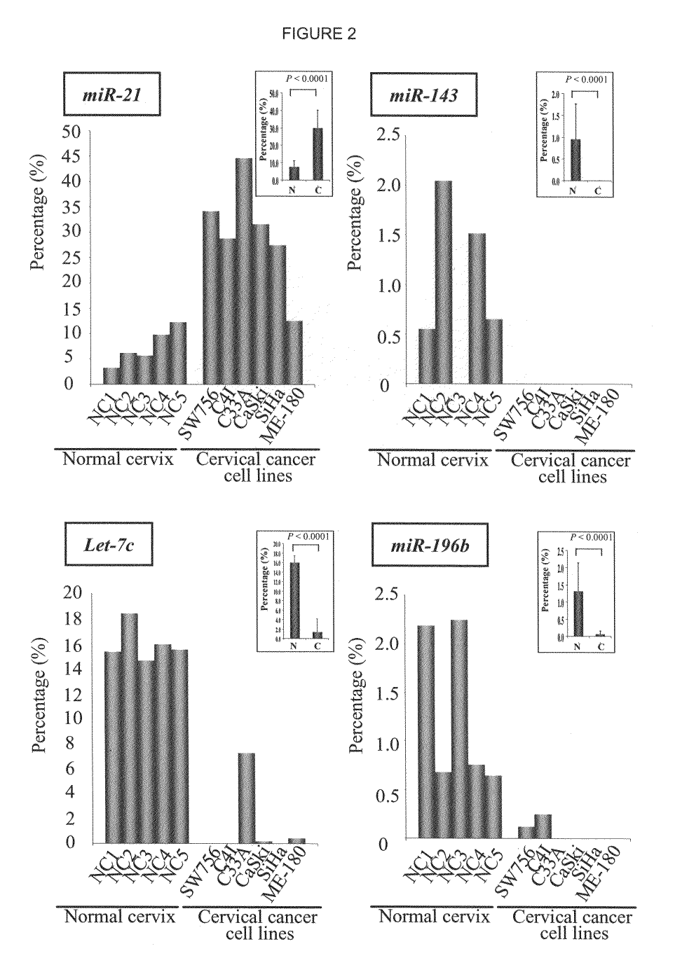

We determined the significance of expression variation between normal cervix and cancer cell lines using χ² test (Table 8). Of 166 miRNAs expressed in normal cervix and cancer cell lines, six were found to have substantial expression variation between the two groups (Table 9). While Let-7b, let-7c, miR-23b, miR-196b and miR-143 showed significantly reduced abundance in cervical cancer cell lines, miR-21 displayed higher abundance in the cancer group (FIG. 2).

TABLE 8

| | Normal cervix | | Cervical cancer cell lines | | Normal cervix | | Cervical cancer cell lines | | |
|---|---|---|---|---|---|---|---|---|---|
| | Present | Absent | Present | Absent | Present | Absent | Present | Absent | |
| miRNA | Obs | Obs | Obs | Obs | Exp | Exp | Exp | Exp | p-value* |
| let-7c | 113 | 593 | 21 | 3210 | 24.0 | 682.0 | 110.0 | 3194.0 | 0.0000 |
| miR-21 | 52 | 654 | 956 | 2275 | 180.8 | 525.2 | 827.2 | 2298.1 | 0.0000 |
| let-7b | 124 | 582 | 318 | 2913 | 79.3 | 626.7 | 362.7 | 2905.0 | 0.0000 |
| miR-196b | 9 | 697 | 3 | 3228 | 2.2 | 703.8 | 9.8 | 3226.8 | 0.0000 |
| miR-143 | 7 | 699 | | 3231 | 1.3 | 704.7 | 5.7 | 3230.0 | 0.0000 |
| miR-23b | 18 | 688 | 20 | 3211 | 6.8 | 699.2 | 31.2 | 3209.0 | 0.0001 |
| miR-200c | 57 | 649 | 143 | 3088 | 35.9 | 670.1 | 164.1 | 3084.2 | 0.0012 |
| miR-126-3p | 3 | 703 | | 3231 | 0.5 | 705.5 | 2.5 | 3230.6 | 0.0103 |
| miR-99a | 6 | 700 | 5 | 3226 | 2.0 | 704.0 | 9.0 | 3225.3 | 0.0182 |
| miR-374 | 3 | 703 | 1 | 3230 | 0.7 | 705.3 | 3.3 | 3229.6 | 0.0312 |
| miR-142-3p | 2 | 704 | | 3231 | 0.4 | 705.6 | 1.6 | 3230.7 | 0.0572 |
| miR-145 | 2 | 704 | | 3231 | 0.4 | 705.6 | 1.6 | 3230.7 | 0.0572 |
| miR-154-5p | 2 | 704 | | 3231 | 0.4 | 705.6 | 1.6 | 3230.7 | 0.0572 |
| miR-199a-5p | 2 | 704 | | 3231 | 0.4 | 705.6 | 1.6 | 3230.7 | 0.0572 |
| miR-495 | 2 | 704 | | 3231 | 0.4 | 705.6 | 1.6 | 3230.7 | 0.0572 |
| miR-92 | 14 | 692 | 28 | 3203 | 7.5 | 698.5 | 34.5 | 3201.8 | 0.0775 |
| miR-23a | 15 | 691 | 126 | 3105 | 25.3 | 680.7 | 115.7 | 3106.8 | 0.1541 |
| miR-22 | 2 | 704 | 1 | 3230 | 0.5 | 705.5 | 2.5 | 3229.7 | 0.1835 |
| miR-30a-3p | 1 | 705 | | 3231 | 0.2 | 705.8 | 0.8 | 3230.9 | 0.2890 |
| miR-33 | 1 | 705 | | 3231 | 0.2 | 705.8 | 0.8 | 3230.9 | 0.2890 |
| miR-124a | 1 | 705 | | 3231 | 0.2 | 705.8 | 0.8 | 3230.9 | 0.2890 |
| miR-127 | 1 | 705 | | 3231 | 0.2 | 705.8 | 0.8 | 3230.9 | 0.2890 |
| miR-128b | 1 | 705 | | 3231 | 0.2 | 705.8 | 0.8 | 3230.9 | 0.2890 |
| miR-133a | 1 | 705 | | 3231 | 0.2 | 705.8 | 0.8 | 3230.9 | 0.2890 |
| miR-140 | 1 | 705 | | 3231 | 0.2 | 705.8 | 0.8 | 3230.9 | 0.2890 |
| miR-146a | 1 | 705 | | 3231 | 0.2 | 705.8 | 0.8 | 3230.9 | 0.2890 |
| miR-181a* | 1 | 705 | | 3231 | 0.2 | 705.8 | 0.8 | 3230.9 | 0.2890 |
| miR-181d | 1 | 705 | | 3231 | 0.2 | 705.8 | 0.8 | 3230.9 | 0.2890 |
| miR-199b-5p | 1 | 705 | | 3231 | 0.2 | 705.8 | 0.8 | 3230.9 | 0.2890 |
| miR-214 | 1 | 705 | | 3231 | 0.2 | 705.8 | 0.8 | 3230.9 | 0.2890 |
| miR-219 | 1 | 705 | | 3231 | 0.2 | 705.8 | 0.8 | 3230.9 | 0.2890 |
| miR-299-3p | 1 | 705 | | 3231 | 0.2 | 705.8 | 0.8 | 3230.9 | 0.2890 |
| miR-328 | 1 | 705 | | 3231 | 0.2 | 705.8 | 0.8 | 3230.9 | 0.2890 |
| miR-368 | 1 | 705 | | 3231 | 0.2 | 705.8 | 0.8 | 3230.9 | 0.2890 |
| miR-375 | 1 | 705 | | 3231 | 0.2 | 705.8 | 0.8 | 3230.9 | 0.2890 |
| miR-376a | 1 | 705 | | 3231 | 0.2 | 705.8 | 0.8 | 3230.9 | 0.2890 |
| miR-381 | 1 | 705 | | 3231 | 0.2 | 705.8 | 0.8 | 3230.9 | 0.2890 |
| miR-423 | 1 | 705 | | 3231 | 0.2 | 705.8 | 0.8 | 3230.9 | 0.2890 |
| miR-424 | 1 | 705 | | 3231 | 0.2 | 705.8 | 0.8 | 3230.9 | 0.2890 |
| miR-450 | 1 | 705 | | 3231 | 0.2 | 705.8 | 0.8 | 3230.9 | 0.2890 |
| miR-494 | 1 | 705 | | 3231 | 0.2 | 705.8 | 0.8 | 3230.9 | 0.2890 |
| miR-542-3p | 1 | 705 | | 3231 | 0.2 | 705.8 | 0.8 | 3230.9 | 0.2890 |
| miR-125b | 18 | 688 | 50 | 3181 | 12.2 | 693.8 | 55.8 | 3180.0 | 0.3316 |
| miR-125a | 8 | 698 | 70 | 3161 | 14.0 | 692.0 | 64.0 | 3162.1 | 0.3654 |
| let-7e | 3 | 703 | 35 | 3196 | 6.8 | 699.2 | 31.2 | 3196.7 | 0.4535 |
| miR-16 | 1 | 705 | 19 | 3212 | 3.6 | 702.4 | 16.4 | 3212.5 | 0.5159 |
| miR-26a | 9 | 697 | 23 | 3208 | 5.7 | 700.3 | 26.3 | 3207.4 | 0.5175 |
| miR-205 | 10 | 696 | 75 | 3156 | 15.2 | 690.8 | 69.8 | 3156.9 | 0.5247 |
| miR-29b | 3 | 703 | 32 | 3199 | 6.3 | 699.7 | 28.7 | 3199.6 | 0.5520 |
| miR-191-5p | 3 | 703 | 32 | 3199 | 6.3 | 699.7 | 28.7 | 3199.6 | 0.5520 |
| miR-27b | 2 | 704 | 3 | 3228 | 0.9 | 705.1 | 4.1 | 3227.8 | 0.6467 |
| miR-455-3p | 2 | 704 | 3 | 3228 | 0.9 | 705.1 | 4.1 | 3227.8 | 0.6467 |
| miR-100 | 6 | 700 | 15 | 3216 | 3.8 | 702.2 | 17.2 | 3215.6 | 0.6543 |
| miR-151-3p | 1 | 705 | 1 | 3230 | 0.4 | 705.6 | 1.6 | 3229.9 | 0.7060 |
| miR-18a | 1 | 705 | 14 | 3217 | 2.7 | 703.3 | 12.3 | 3217.3 | 0.7297 |
| miR-93 | | 706 | 29 | 3202 | 5.2 | 700.8 | 23.8 | 3202.9 | 0.7590 |
| miR-98 | 1 | 705 | 13 | 3218 | 2.5 | 703.5 | 11.5 | 3218.3 | 0.7745 |
| miR-20a | | 706 | 25 | 3206 | 4.5 | 701.5 | 20.5 | 3206.8 | 0.7992 |
| miR-130a | | 706 | 25 | 3206 | 4.5 | 701.5 | 20.5 | 3206.8 | 0.7992 |
| miR-21* | 2 | 704 | 4 | 3227 | 1.1 | 704.9 | 4.9 | 3226.8 | 0.8089 |
| miR-31 | 1 | 705 | 12 | 3219 | 2.3 | 703.7 | 10.7 | 3219.2 | 0.8185 |
| miR-200b | 1 | 705 | 12 | 3219 | 2.3 | 703.7 | 10.7 | 3219.2 | 0.8185 |
| miR-15b | 2 | 704 | 18 | 3213 | 3.6 | 702.4 | 16.4 | 3213.3 | 0.8354 |
| miR-103 | | 706 | 21 | 3210 | 3.8 | 702.2 | 17.2 | 3210.7 | 0.8391 |
| miR-24 | | 706 | 20 | 3211 | 3.6 | 702.4 | 16.4 | 3211.6 | 0.8490 |
| miR-27a | 4 | 702 | 29 | 3202 | 5.9 | 700.1 | 27.1 | 3202.3 | 0.8584 |
| let-7a | 57 | 649 | 231 | 3000 | 51.6 | 654.4 | 236.4 | 2999.0 | 0.8683 |
| miR-99b | 5 | 701 | 15 | 3216 | 3.6 | 702.4 | 16.4 | 3215.7 | 0.8775 |
| let-7d | 6 | 700 | 39 | 3192 | 8.1 | 697.9 | 36.9 | 3192.4 | 0.8842 |
| miR-30c | | 706 | 16 | 3215 | 2.9 | 703.1 | 13.1 | 3215.5 | 0.8875 |
| miR-106b | | 706 | 16 | 3215 | 2.9 | 703.1 | 13.1 | 3215.5 | 0.8875 |
| miR-92b | | 706 | 13 | 3218 | 2.3 | 703.7 | 10.7 | 3218.4 | 0.9151 |
| miR-25 | 5 | 701 | 16 | 3215 | 3.8 | 702.2 | 17.2 | 3214.8 | 0.9200 |
| miR-148b | 1 | 705 | 2 | 3229 | 0.5 | 705.5 | 2.5 | 3228.9 | 0.9224 |
| miR-324-5p | 1 | 705 | 2 | 3229 | 0.5 | 705.5 | 2.5 | 3228.9 | 0.9224 |
| miR-425-5p | 1 | 705 | 2 | 3229 | 0.5 | 705.5 | 2.5 | 3228.9 | 0.9224 |
| miR-505 | 1 | 705 | 2 | 3229 | 0.5 | 705.5 | 2.5 | 3228.9 | 0.9224 |
| miR-30a-5p | | 706 | 11 | 3220 | 2.0 | 704.0 | 9.0 | 3220.4 | 0.9326 |
| miR-30b | | 706 | 11 | 3220 | 2.0 | 704.0 | 9.0 | 3220.4 | 0.9326 |
| miR-320 | 4 | 702 | 26 | 3205 | 5.4 | 700.6 | 24.6 | 3205.2 | 0.9332 |
| miR-181b | 1 | 705 | 9 | 3222 | 1.8 | 704.2 | 8.2 | 3222.1 | 0.9343 |
| miR-182-5p | 1 | 705 | 9 | 3222 | 1.8 | 704.2 | 8.2 | 3222.1 | 0.9343 |
| miR-193b | | 706 | 10 | 3221 | 1.8 | 704.2 | 8.2 | 3221.3 | 0.9410 |
| let-7f | 28 | 678 | 145 | 3086 | 31.0 | 675.0 | 142.0 | 3086.5 | 0.9458 |
| let-7g | 7 | 699 | 41 | 3190 | 8.6 | 697.4 | 39.4 | 3190.3 | 0.9465 |
| miR-221 | | 706 | 9 | 3222 | 1.6 | 704.4 | 7.4 | 3222.3 | 0.9491 |
| let-7i | 5 | 701 | 30 | 3201 | 6.3 | 699.7 | 28.7 | 3201.2 | 0.9565 |
| miR-28 | | 706 | 8 | 3223 | 1.4 | 704.6 | 6.6 | 3223.3 | 0.9569 |
| miR-183 | | 706 | 8 | 3223 | 1.4 | 704.6 | 6.6 | 3223.3 | 0.9569 |
| miR-424-3p | 1 | 705 | 8 | 3223 | 1.6 | 704.4 | 7.4 | 3223.1 | 0.9628 |
| miR-30d | | 706 | 7 | 3224 | 1.3 | 704.7 | 5.7 | 3224.2 | 0.9644 |
| miR-26b | 2 | 704 | 6 | 3225 | 1.4 | 704.6 | 6.6 | 3224.9 | 0.9652 |
| miR-342 | | 706 | 6 | 3225 | 1.1 | 704.9 | 4.9 | 3225.2 | 0.9714 |
| miR-29a | 6 | 700 | 34 | 3197 | 7.2 | 698.8 | 32.8 | 3197.2 | 0.9716 |
| miR-101 | | 706 | 5 | 3226 | 0.9 | 705.1 | 4.1 | 3226.2 | 0.9781 |
| miR-181a | | 706 | 5 | 3226 | 0.9 | 705.1 | 4.1 | 3226.2 | 0.9781 |
| miR-15a | | 706 | 4 | 3227 | 0.7 | 705.3 | 3.3 | 3227.1 | 0.9841 |
| miR-29b* | | 706 | 4 | 3227 | 0.7 | 705.3 | 3.3 | 3227.1 | 0.9841 |
| miR-106b* | | 706 | 4 | 3227 | 0.7 | 705.3 | 3.3 | 3227.1 | 0.9841 |
| miR-125a* | | 706 | 4 | 3227 | 0.7 | 705.3 | 3.3 | 3227.1 | 0.9841 |
| miR-186 | | 706 | 4 | 3227 | 0.7 | 705.3 | 3.3 | 3227.1 | 0.9841 |
| miR-193a | | 706 | 4 | 3227 | 0.7 | 705.3 | 3.3 | 3227.1 | 0.9841 |
| miR-193b* | | 706 | 4 | 3227 | 0.7 | 705.3 | 3.3 | 3227.1 | 0.9841 |
| miR-224 | | 706 | 4 | 3227 | 0.7 | 705.3 | 3.3 | 3227.1 | 0.9841 |
| miR-331 | | 706 | 4 | 3227 | 0.7 | 705.3 | 3.3 | 3227.1 | 0.9841 |
| miR-423* | | 706 | 4 | 3227 | 0.7 | 705.3 | 3.3 | 3227.1 | 0.9841 |
| miR-532 | | 706 | 4 | 3227 | 0.7 | 705.3 | 3.3 | 3227.1 | 0.9841 |
| miR-19b | | 706 | 3 | 3228 | 0.5 | 705.5 | 2.5 | 3228.1 | 0.9896 |
| miR-130b-3p | | 706 | 3 | 3228 | 0.5 | 705.5 | 2.5 | 3228.1 | 0.9896 |
| miR-452 | | 706 | 3 | 3228 | 0.5 | 705.5 | 2.5 | 3228.1 | 0.9896 |
| miR-151-5p | 22 | 684 | 93 | 3138 | 20.6 | 685.4 | 94.4 | 3137.8 | 0.9900 |
| miR-574 | 2 | 704 | 7 | 3224 | 1.6 | 704.4 | 7.4 | 3223.9 | 0.9903 |
| let-7b* | | 706 | 2 | 3229 | 0.4 | 705.6 | 1.6 | 3229.1 | 0.9943 |
| let-7i* | | 706 | 2 | 3229 | 0.4 | 705.6 | 1.6 | 3229.1 | 0.9943 |
| miR-7 | | 706 | 2 | 3229 | 0.4 | 705.6 | 1.6 | 3229.1 | 0.9943 |
| miR-17-3p | | 706 | 2 | 3229 | 0.4 | 705.6 | 1.6 | 3229.1 | 0.9943 |
| miR-19a | | 706 | 2 | 3229 | 0.4 | 705.6 | 1.6 | 3229.1 | 0.9943 |
| miR-20b | | 706 | 2 | 3229 | 0.4 | 705.6 | 1.6 | 3229.1 | 0.9943 |
| miR-23a* | | 706 | 2 | 3229 | 0.4 | 705.6 | 1.6 | 3229.1 | 0.9943 |
| miR-27b-5p | | 706 | 2 | 3229 | 0.4 | 705.6 | 1.6 | 3229.1 | 0.9943 |
| miR-28* | | 706 | 2 | 3229 | 0.4 | 705.6 | 1.6 | 3229.1 | 0.9943 |
| miR-29c | | 706 | 2 | 3229 | 0.4 | 705.6 | 1.6 | 3229.1 | 0.9943 |
| miR-30a-5p | | 706 | 2 | 3229 | 0.4 | 705.6 | 1.6 | 3229.1 | 0.9943 |
| miR-96 | | 706 | 2 | 3229 | 0.4 | 705.6 | 1.6 | 3229.1 | 0.9943 |
| miR-140* | | 706 | 2 | 3229 | 0.4 | 705.6 | 1.6 | 3229.1 | 0.9943 |
| miR-149-5p | | 706 | 2 | 3229 | 0.4 | 705.6 | 1.6 | 3229.1 | 0.9943 |
| miR-196a | | 706 | 2 | 3229 | 0.4 | 705.6 | 1.6 | 3229.1 | 0.9943 |
| miR-218 | | 706 | 2 | 3229 | 0.4 | 705.6 | 1.6 | 3229.1 | 0.9943 |
| miR-342* | | 706 | 2 | 3229 | 0.4 | 705.6 | 1.6 | 3229.1 | 0.9943 |
| miR-210 | 1 | 705 | 6 | 3225 | 1.3 | 704.7 | 5.7 | 3225.0 | 0.9958 |
| miR-222 | 1 | 705 | 6 | 3225 | 1.3 | 704.7 | 5.7 | 3225.0 | 0.9958 |
| miR-10a | | 706 | 1 | 3230 | 0.2 | 705.8 | 0.8 | 3230.0 | 0.9980 |
| miR-10a | | 706 | 1 | 3230 | 0.2 | 705.8 | 0.8 | 3230.0 | 0.9980 |
| miR-15b* | | 706 | 1 | 3230 | 0.2 | 705.8 | 0.8 | 3230.0 | 0.9980 |
| miR-18a* | | 706 | 1 | 3230 | 0.2 | 705.8 | 0.8 | 3230.0 | 0.9980 |
| miR-23b* | | 706 | 1 | 3230 | 0.2 | 705.8 | 0.8 | 3230.0 | 0.9980 |
| miR-92* | | 706 | 1 | 3230 | 0.2 | 705.8 | 0.8 | 3230.0 | 0.9980 |
| miR-101* | | 706 | 1 | 3230 | 0.2 | 705.8 | 0.8 | 3230.0 | 0.9980 |
| miR-130b-5p | | 706 | 1 | 3230 | 0.2 | 705.8 | 0.8 | 3230.0 | 0.9980 |
| miR-132 | | 706 | 1 | 3230 | 0.2 | 705.8 | 0.8 | 3230.0 | 0.9980 |
| miR-135b | | 706 | 1 | 3230 | 0.2 | 705.8 | 0.8 | 3230.0 | 0.9980 |
| miR-138 | | 706 | 1 | 3230 | 0.2 | 705.8 | 0.8 | 3230.0 | 0.9980 |
| miR-141 | | 706 | 1 | 3230 | 0.2 | 705.8 | 0.8 | 3230.0 | 0.9980 |
| miR-149-3p | | 706 | 1 | 3230 | 0.2 | 705.8 | 0.8 | 3230.0 | 0.9980 |
| miR-152 | | 706 | 1 | 3230 | 0.2 | 705.8 | 0.8 | 3230.0 | 0.9980 |
| miR-193a* | | 706 | 1 | 3230 | 0.2 | 705.8 | 0.8 | 3230.0 | 0.9980 |
| miR-197 | | 706 | 1 | 3230 | 0.2 | 705.8 | 0.8 | 3230.0 | 0.9980 |
| miR-296 | | 706 | 1 | 3230 | 0.2 | 705.8 | 0.8 | 3230.0 | 0.9980 |
| miR-324-3p | | 706 | 1 | 3230 | 0.2 | 705.8 | 0.8 | 3230.0 | 0.9980 |
| miR-330* | | 706 | 1 | 3230 | 0.2 | 705.8 | 0.8 | 3230.0 | 0.9980 |
| miR-335 | | 706 | 1 | 3230 | 0.2 | 705.8 | 0.8 | 3230.0 | 0.9980 |
| miR-338* | | 706 | 1 | 3230 | 0.2 | 705.8 | 0.8 | 3230.0 | 0.9980 |
| miR-429 | | 706 | 1 | 3230 | 0.2 | 705.8 | 0.8 | 3230.0 | 0.9980 |
| mi&-455-5p | | 706 | 1 | 3230 | 0.2 | 705.8 | 0.8 | 3230.0 | 0.9980 |
| miR-484 | | 706 | 1 | 3230 | 0.2 | 705.8 | 0.8 | 3230.0 | 0.9980 |
| miR-491 | | 706 | 1 | 3230 | 0.2 | 705.8 | 0.8 | 3230.0 | 0.9980 |
| miR-522 | | 706 | 1 | 3230 | 0.2 | 705.8 | 0.8 | 3230.0 | 0.9980 |
| miR-542-5p | | 706 | 1 | 3230 | 0.2 | 705.8 | 0.8 | 3230.0 | 0.9980 |
| miR-582-3p | | 706 | 1 | 3230 | 0.2 | 705.8 | 0.8 | 3230.0 | 0.9980 |
| miR-584 | | 706 | 1 | 3230 | 0.2 | 705.8 | 0.8 | 3230.0 | 0.9980 |
| miR-155 | 1 | 705 | 4 | 3227 | 0.9 | 705.1 | 4.1 | 3227.0 | 0.9995 |
| miR-365 | 1 | 705 | 4 | 3227 | 0.9 | 705.1 | 4.1 | 3227.0 | 0.9995 |
| miR-34a | 1 | 705 | 5 | 3226 | 1.1 | 704.9 | 4.9 | 3226.0 | 0.9999 |
| miR-200a | 1 | 705 | 5 | 3226 | 1.1 | 704.9 | 4.9 | 3226.0 | 0.9999 |
| miR-17-5p | 6 | 700 | 27 | 3204 | 5.9 | 700.1 | 27.1 | 3204.0 | 1.0000 |
| miR-185 | 3 | 703 | 14 | 3217 | 3.0 | 703.0 | 14.0 | 3217.0 | 1.0000 |

*p-value was calculated using χ2 test; p < 0.0001 is considered as significant.

TABLE 9
Table S6. miRNAs with significant expression variation
between normal cervix and cervical cancer cell lines

| | Normal cervix | | Cervical cancer cell lines | | |
|---|---|---|---|---|---|
| miRNA | No. (n = 706) | % | No. (n = 3231) | % | p-value* |
| Let-7b | 124 | 17.6 | 318 | 9.8 | <0.0001 |
| Let-7c | 113 | 16.0 | 21 | 0.6 | <0.0001 |
| miR-21 | 52 | 7.4 | 956 | 29.6 | <0.0001 |
| miR-23b | 18 | 2.5 | 20 | 0.6 | <0.0001 |
| miR-196b | 9 | 1.3 | 3 | 0.1 | <0.0001 |
| miR-143 | 7 | 1.0 | 0 | 0 | <0.0001 |

*p-value was calculated using χ² test; p < 0.0001 is considered as significant.

For let-7c, a frequency of 15-18% (16.0±1.3) in individual normal cervical samples, compares with no detection in three of the cancer cell lines, 0.2-0.4% in two lines and 7.2% in one line. For miR-21, 13-45% (29.8±10.5) were found in the cancer group, while 3-12% (7.4±3.6) were identified in the normal cervix. The cloning frequency for miR-196b ranges from 1-2% (1.3±0.8) in normal cervical samples, while only 0.1% (3 clones out of 3231) was detected in cancer cell lines. For miR-143, none was found in the cancer group (0/3231), while a total of 7 (out of 706) were detected in the normal group.

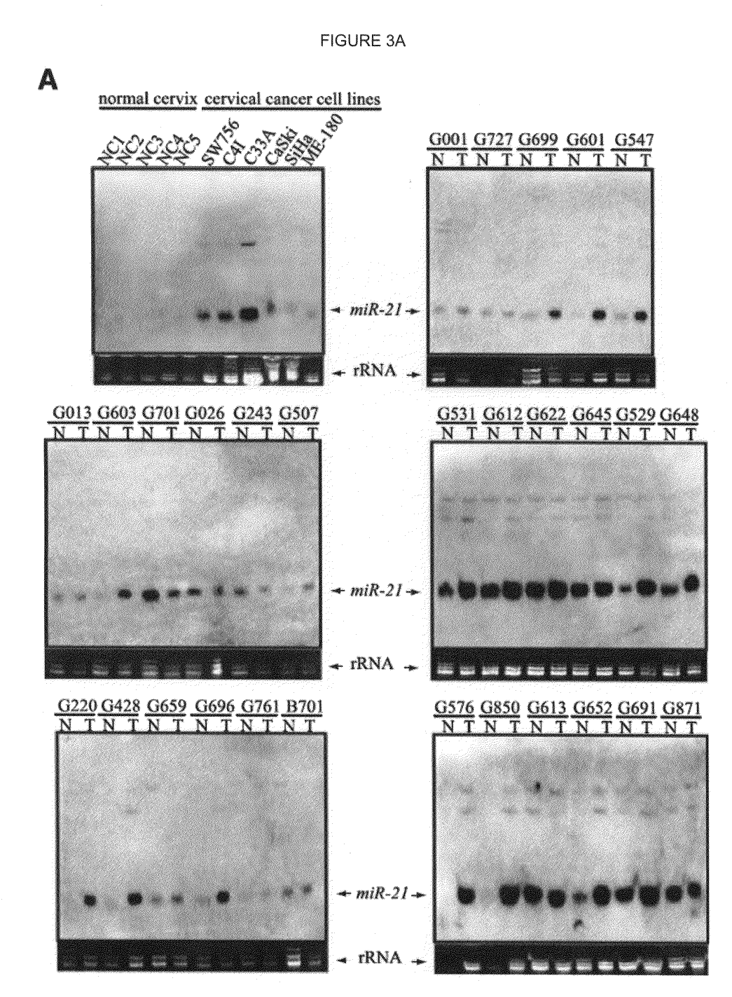

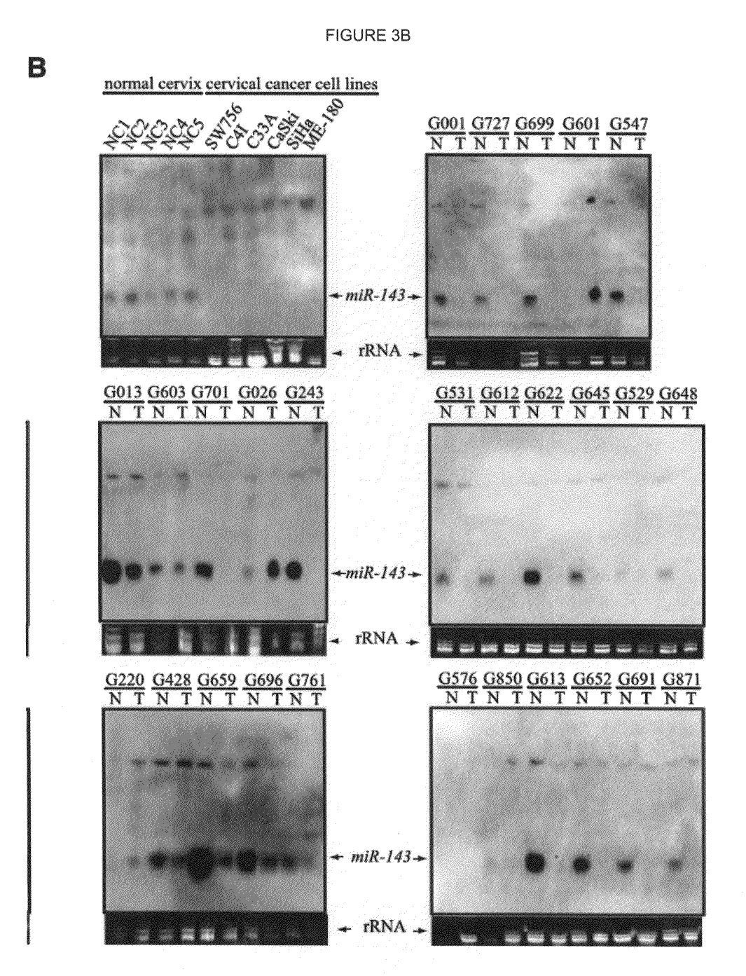

Verification of miRNA expression variations by Northern analysis. To further investigate the miRNA expression variations suggested by cloning, we evaluated the expression level of miR-21 and miR-143 in all normal cervix and cancer cell lines used in the cloning experiments. The Northern results revealed that the expression of both miR-21 and miR-143 was significantly different between the normal cervix and cervical cancer cell lines (FIG. 3). In agreement with the cloning data, accumulation of miR-21 was substantially greater in cervical cancer cell lines, while miR-143 was substantially more abundant in normal cervix.

mir-21 and mir-143 expression in cervical tumors and matched controls. We extended the Northern blot analysis of miR-21 and miR-143 on a series of 29 cervical cancer samples and matched normal cervices (control samples from the same patients) provided by the Columbus (Ohio) Children's Hospital Gynecologic Oncology Group Tissue Bank. The results are summarized in Table 10. For miR-21, expression levels were clearly higher in 21 of the 29 tumor samples compared to their normal counterparts (FIG. 3A). In two cases (G613 and G871), mir-21 expression appeared comparable between the cancer and normal tissues. The remaining six cases were undetermined due to poor detection or uneven RNA yields for the matched samples. For miR-143, the expression patterns can be divided into several categories (FIG. 3B): (i) absent or barely detectable expression in the tumor samples with substantial expression detected in the matched normal samples (16 of 27 samples tested), (ii) lower (but detectable) expression level in the tumor with strong expression in normal counterpart tissue (5 of 27 samples tested), and (iii) more abundant expression level in the tumor than that in its normal matched sample (2 of 27 samples tested). The remaining four cases were undetermined (see above).

TABLE 10

| | | |
|---|---|---|
| Sample | Age | Northern blot analysis* |

| ID | (years) | Diagnosis | miR-21 | miR-143 |
|---|---|---|---|---|
| G013 | 53 | SCC | ↑ | ↓↓↓ |
| G026 | 62 | SCC | ND | ↑↑↑ |
| G243 | 30 | SCC | ND | ↓↓↓ |
| G507 | 52 | SCC | ↑ | NA |
| G529 | NA | SCC | ↑↑↑ | ND |
| G531 | 49 | SCC | ↑↑↑ | ↓↓↓ |
| G576 | 48 | SCC | ND | ND |
| G601 | 55 | SCC | ↑↑↑ | ↑↑↑ |
| G603 | 48 | SCC | ↑↑↑ | ↓↓ |
| G612 | NA | SCC | ↑↑ | ↓↓↓ |
| G613 | 48 | SCC | NC | ↓↓↓ |
| G622 | 35 | SCC | ↑↑ | ↓↓↓ |
| G645 | 70 | SCC | ↑↑ | ↓↓↓ |
| G648 | NA | SCC | ↑↑↑ | ↓ |
| G652 | 46 | SCC | ↑↑↑ | ↓↓↓ |
| G699 | 57 | SCC | ↑↑↑ | ↓↓↓ |
| G701 | 25 | SCC | ND | ↓↓↓ |
| G727 | NA | SCC | ↑ | ↓ |
| G850 | 50 | SCC | ND | ND |
| G220 | NA | ADC | ↑↑↑ | ND |
| G428 | 38 | ADC | ↑↑↑ | ↓ |
| G547 | 60 | ADC | ↑↑↑ | ↓↓↓ |
| G659 | NA | ADC | ↑ | ↓↓ |
| G691 | 29 | ADC | ↑↑ | ↓↓ |
| G696 | NA | ADC | ↑↑↑ | ↓↓ |
| G761 | NA | ADC | ↑ | ↓↓ |
| B701 | 33 | ADSC | ND | NA |
| G871 | 47 | ADSC | NC | ↓↓ |
| G001 | NA | SmC | ↑ | ↓↓↓ |

SCC, squamous cell carcinoma; ADC, adenocarcinoma; ADSC, adenosquamous cell carcinoma; SmC, small cell carcinoma;
*Relative abundance level in tumor sample as compared to its normal tissue counterpart;
↑, increased; ↓, decreased; ND, not determined; NC, no change; NA, not available.

### Discussion

Cervical cancer, the second most common cancer amongst women worldwide, is frequently caused by specific human papillomavirus (HPV) infections. Although participation of HPV proteins (e.g. E6 and E7) in cell transformation is well documented, the detailed network of events leading from HPV infection to tumor development has yet to be elucidated. Given that numerous viruses (both DNA and RNA) have been shown to encode small modulatory RNAs, and given the changes in endogenous miRNA pattern and tumor-related roles for miRNAs that have been demonstrated, we thought it appropriate to investigate small RNA profiles in a set of well-characterized cervical cancer cell lines, and compared the profiles with normal cervical specimens.

In order to obtain a definitive and inclusive profile, we used a cloning-based method for profiling RNAs that makes no initial assumptions about the identity of the small RNAs present. Applying this protocol, we found that known miRNAs accounted for a large proportion of small RNAs expressed in each of the cervical cancer cell lines and normal cervix. In addition to these known miRNAs, we identified a number of novel small RNAs with miRNA characteristics, which had not been previously observed in any tissue or cell type. Finally, we identified a significant number of novel small RNAs which do not meet standard miRNA criteria and which may thus be synthesized by distinct mechanisms.

Our analysis of profiles for known miRNAs indicated significant similarities and differences of expression level between the cell lines and normal cervical samples. At least six miRNAs showed substantial expression variations between the two groups, in which the differential expression pattern of two miRNAs were also corroborated by Northern blot analysis. These findings led us to examine the biological relevance of these miRNAs in cervical cancer using a panel of clinical samples. Remarkably, the differential expression pattern of these two miRNAs is consistent in tumors, thus raising the possibility that these miRNAs (or their targets) may provide useful diagnostic tools and may also provide clues to understand the processes that lead to cancer development.

Increased expression of miR-21 in cervical cancer. miR-21 was the most abundantly recovered miRNA species in all of the cervical cancer cell lines we examined, showing significantly lower abundance in the normal cervical samples analyzed. Concordantly, the increased expression of miR-21 was also found in a preponderance of tumors. Abundant miR-21 may be a general, albeit not universal, feature of tumor cells. In addition to our analysis, strong miR-21 signals have been reported in several hybridization-based profiles of different tumor types (e.g. glioblastomas, breast cancer) and cancer cell lines (e.g. HCT-116, colorectal carcinoma; HeLa, cervical adenocarcinoma, and glioblastoma cell lines). Interestingly, miR-21 is located in the fragile site FRA17B region, which is one of the HPV16 integration loci at 17q23.2. It is known that HPV integration into the host cell genome can cause genetic alterations (such as deletions, amplifications or complex rearrangements) and epigenetic alteration, thus the expression of cellular miRNA genes at or near HPV integration sites may contribute to the tumor phenotype.

Reduced expression of miR-143 in cervical cancer. The abundance level of miR-143 is strikingly different between normal cervical samples and cervical cancer cell lines, which led us to investigate its association with cervical cancer development. In agreement with the cloning data, the expression of miR-143 is significantly lower in most of the tumors as compared to their normal counterparts. Similarly, mature miR-143 is significantly reduced in different tumor types, e.g. colorectal tumors, sarcomas, breast, prostate and lymphoid cancer cell lines, suggesting that miR-143 may have suppressor roles in a wide range of tumor cells. The only experimentally verified target for miR-143, ERK5 (also known as MAPK7), is known to promote cell growth and proliferation in response to tyrosine kinase signaling. Furthermore, abnormal levels of ERK5 expression have been observed in some cancer types, suggesting its significant role in tumor development.

No detection of HPV-derived miRNAs. One conceivable result from our analysis would have been the identification of HPV-encoded miRNAs. The cell lines included in this study consisted of five HPV-positive cervical cancer cell lines (SW756, C4I, CaSki, SiHa and ME-180) and one apparently HPV-negative cell line (C33A). The detection of the HPV genome for these cell lines had been reported, and was re-confirmed by DNA sequencing. We did not identify any viral-encoded miRNAs from the HPV-infected cells by our molecular cloning approach, even after screening ˜4000 clones. Consistent with this observation, no viral-encoded miRNA was predicted from the HPV18 genome by a computational method and no viral derived small RNAs were detected during latent or productive replication cycle of HPV31 by a cloning approach.

Novel miRNAs and other small RNAs. Among the small RNAs cloned from the cervical cancer cell lines and normal cervical samples, we observed a moderate number of previously unconfirmed or unidentified transcripts, which appear by all criteria to be novel miRNAs. The identification of novel miRNAs and other novel sRNAs in these specific tumor cell lines and cell type indicates that some of these small RNAs may be unique to this cell type.

Although one might expect some novel miRNAs to be expressed specifically in one tumor type, it is conceivable that others would be expressed in a range of tumor types. Interestingly we have found several of the novel miRNAs in diverse tumor types. In particular, miR-935, miR-708 and miR-874-3p were also found in human sarcomas; and miR-940 was also found in a renal cell carcinoma cell line.

Novel small RNAs that do not meet current miRNA criteria could represent various situations: miRNAs formed by a standard mechanism but failing the arbitrary miRNA criteria due to incompleteness of these criteria, miRNA-like molecules formed by slightly divergent synthetic mechanisms, and other small RNAs such as natural siRNAs that might be formed by completely different mechanisms. Finally, these could represent spurious ssRNA transcripts or common degradation products of longer cellular RNAs. Of these, sRNA-cer3 was also found in a colon cancer cell line and sRNA-14 was also observed in a renal cell carcinoma cell line, indicating that some of these small RNAs are not specific to cervical cancer. Given that tumor genomes use every conceivable mechanism to modulate their own gene expression for the promotion of tumor growth, it would be surprising not to have the siRNA pathway utilized during tumorigenesis. Further analysis will certainly be required to assess the biological effect of these small molecules. Although the individual small non-miRNAs are rare in cervical cancer cells, their low concentration alone does not rule out their possible biological roles. In particular, an analysis of RNAi effects in C. elegans by Pak and Fire Science 2007; 315:241-4 show that substantial interference effects can be initiated by a population of siRNAs that are only marginally detectable in cloning experiments.

Prospects for miRNA discovery and profiling. Since the immense potential of miRNAs as regulators of gene networks is just beginning to unfold, a detailed molecular analysis of miRNA expression in the process of cancer development is of great interest. As accumulating evidence showing the clinical impacts of miRNA expression profiles, a more comprehensive list of miRNAs needs to be identified for cataloging of expression patterns in specific tissue types in physiological and pathological conditions. Therefore, the small RNA cloning approach (for example as described by Margulies et al. (2005) Nature 437:376-80), should enable the enumeration and characterization of a more complete set of human miRNAs for subsequent analysis.

What is claimed is:

1. A method for the diagnosis or staging of cancer, the method comprising:
determining the upregulation or downregulation of expression of a small RNA sequence.

2. The method according to claim 1, wherein said cervical cancer is squamous cell carcinoma of the cervix.

3. The method according to claim 1, wherein said determining comprises detecting increased or decreased amounts of mRNA corresponding to a differentially expressed small RNA in a sample of cervical cells.

4. The method according to claim 1, wherein expression is determined by real time PCR.

5. The method according to claim 4, wherein said PCR is performed with a set of primers.

6. The method according to claim 1, wherein expression is determined by hybridization to an array.

7. The method according to claim 6, wherein said array comprises two or more sequences selected from let-7b, let-7c, miR-23b, miR196b, miR-143 and miR-21.

8. The method according to claim 1, wherein expression is determined by sequencing of populations of small RNAs from samples of cells.

9. The method according to claim 1, wherein expression is determined by sequencing of populations of cDNA molecules derived from small RNAs from samples of cells.

10. The method of claim 1 in which the small RNA sequence is genetic sequence selected from those listed in Table 1, Table 2, Table 3 and Table 9.

11. The method according to claim 10, wherein said cervical cancer is squamous cell carcinoma of the cervix.

12. The method according to claim 10, wherein said determining comprises detecting increased or decreased amounts of mRNA corresponding to a differentially expressed small RNA in a sample of cervical cells.

13. The method according to claim 10, wherein expression is determined by real time PCR.

14. The method according to claim 13, wherein said PCR is performed with a set of primers.

15. The method according to claim 10, wherein expression is determined by hybridization to an array.

16. The method according to claim 15, wherein said array comprises two or more sequences selected from let-7b, let-7c, miR-23b, miR196b, miR-143 and miR-21.

17. The method according to claim 10, wherein expression is determined by sequencing of populations of small RNAs from samples of cells.

18. The method according to claim 10, wherein expression is determined by sequencing of populations of cDNA molecules derived from small RNAs from samples of cells.

19. A method for the diagnosis or staging of cancer, the method comprising:
determining the upregulation or downregulation of expression of a small RNA sequence selected from those listed in Table 1, Table 2, Table 3 and Table 9.

20. The method according to claim 19, wherein said cervical cancer is squamous cell carcinoma of the cervix.

21. The method according to claim 19, wherein said determining comprises detecting increased or decreased amounts of mRNA corresponding to a differentially expressed small RNA in a sample of cervical cells.

22. The method according to claim 19, wherein expression is determined by real time PCR.

23. The method according to claim 22, wherein said PCR is performed with a set of primers.

24. The method according to claim 19, wherein expression is determined by hybridization to an array.

25. The method according to claim 24, wherein said array comprises two or more sequences selected from let-7b, let-7c, miR-23b, miR196b, miR-143 and miR-21.

26. The method according to claim 19, wherein expression is determined by sequencing of populations of small RNAs from samples of cells.

27. The method according to claim 19, wherein expression is determined by sequencing of populations of cDNA molecules derived from small RNAs from samples of cells.

28. A method for the diagnosis or staging of cancer, the method comprising:
determining the upregulation or downregulation of a group of small RNA sequences selected from those listed in Table 1, Table 2, Table 3 and Table 9.

29. The method according to claim 28, wherein said cervical cancer is squamous cell carcinoma of the cervix.

30. The method according to claim 28, wherein said determining comprises detecting increased or decreased amounts of mRNA corresponding to a differentially expressed small RNA in a sample of cervical cells.

31. The method according to claim 28, wherein expression is determined by real time PCR.

32. The method according to claim 31, wherein said PCR is performed with a set of primers.

33. The method according to claim 28, wherein expression is determined by hybridization to an array.

34. The method according to claim 33, wherein said array comprises two or more sequences selected from let-7b, let-7c, miR-23b, miR196b, miR-143 and miR-21.

35. The method according to claim 28, wherein expression is determined by sequencing of populations of small RNAs from samples of cells.

36. The method according to claim 28, wherein expression is determined by sequencing of populations of cDNA molecules derived from small RNAs from samples of cells.

37. A method of screening candidate agents for modulation of a cervical cancer target protein, the method comprising:
combining a candidate biologically active agent with a cell expressing a small RNA sequence selected from those listed in Table 1, Table 2, Table 3 and Table 9; and
determining the effect of said agent on the phenotype of the cervical cancer cell, wherein agents that modulate said phenotype activity provide a candidate therapeutic agent for cervical cancer.

38. The method according to claim 37, wherein said biologically active agent downregulates expression.

39. The method according to claim 37, wherein said biologically active agent upregulates expression.

40. A method of protecting against or treating cancer comprising:
introducing a biologically active agent capable of increasing or decreasing the activity or expression of small RNA sequence selected from those listed in Table 1, Table 2, Table 3 and Table 9 by at least about 1.5-fold, at least about 2-fold, at least about 5-fold, or at least about 10-fold.

41. The method according to claim 40, wherein said biologically active agent downregulates expression.

42. The method according to claim 40, wherein said biologically active agent upregulates expression.

43. A method of protecting against or treating cancer comprising:
introducing a plurality of biologically active agents capable of increasing or decreasing the activity or expression of small RNA sequence selected from those listed in Table 1, Table 2, Table 3 and Table 9 by at least about 1.5-fold, at least about 2-fold, at least about 5-fold, or at least about 10-fold.
